(19) United States
(12) Patent Application Publication
(10) Pub. No.: US 2013/0183282 A1
(43) Pub. Date: Jul. 18, 2013

(54) Meganuclease variants cleaving a DNA target sequence from the rhodopsin gene and uses thereof

(71) Applicants: Frédéric Lemaire, Paris (FR); Sylvain Arnould, Saint-Denis (FR)

(72) Inventors: Frédéric Lemaire, Paris (FR); Sylvain Arnould, Saint-Denis (FR)

(73) Assignee: CELLECTIS, Paris (FR)

(21) Appl. No.: 13/697,614

(22) PCT Filed: May 12, 2011
(86) PCT No.: PCT/IB11/01495

§ 371 Date: Mar. 11, 2013

Related U.S. Application Data
(60) Provisional application No. 61/333,994, filed on May 12, 2010.

(51) Int. Cl. C12N 9/22

(52) U.S. Cl. CPC C12N 9/22
USPC 424/94.6; 435/196; 536/23.2; 435/320.1; 800/13; 800/298; 435/252.3; 435/325; 435/419; 435/254.2

(57) ABSTRACT

The invention relates to meganuclease variants which cleave a DNA target sequence from the human Rhodopsin gene (RHO), to vectors encoding such variants, to a cell, an animal or a plant modified by such vectors and to the use of these meganuclease variants and products derived therefrom for genome therapy, ex vivo (gene cell therapy) and genome engineering including therapeutic applications and cell line engineering.

### BACKGROUND OF THE INVENTION

1. Field of the Invention

The invention relates to meganuclease variants which cleave a DNA target sequence from the human Rhodopsin gene (RHO), to vectors encoding such variants, to a cell, an animal or a plant modified by such vectors and to the use of these meganuclease variants and products derived therefrom for genome therapy, ex vivo (gene cell therapy) and genome engineering including therapeutic applications and cell line engineering.

2. Discussion of the Background Art

Rhodopsin is a member of G protein-coupled receptor (GPCR) family, the largest family of cell surface proteins involved in signaling across membranes that share a common seven alpha-helical transmembrane architecture. Rhodopsin, present in rod photoreceptors, responds to light. The structure of the rod outer segment (ROS), a specialized part of the rod cell containing rhodopsin and auxiliary proteins, allows the very sensitive detection and conversion of light signal.

Mutations in Rhodopsin have been associated with Retinitis pigmentosa (Sullivan et al). Retinitis pigmentosa (RP) is a group of inherited retinal degenerative disorders characterized by progressive degeneration of the midperipheral retina, leading to night blindness, visual field constriction, and eventual loss of visual acuity. RP is one of the leading causes of blindness in adults with an incidence of around 1 in 3,500 worldwide (Hims et al) and therefore this disorder is an important issue to tackle in terms of public health.

RP can be inherited in an autosomal dominant (adRP), recessive (arRP), or x-linked (X-linked retinitis pigmentosa XLRP) manner. According to various reports, adRP represents between 15% and 35% of all RP cases. These values were derived from different studies, with the highest value being found in the United States (Bunker et al.) and the lowest in southern Europe (Ayuso et al). Among about 17 genes that have been identified as causative of adRP, RHO is the most frequently reported adRP gene, contributing to 20%-25% of cases (van Soest et al), or even 26.5% in the USA (Sullivan et al). Therefore, the development of gene therapy methods targeting RHO gene appears valuable to attempt to treat a significant fraction of RP patients, in particular adRP patients for whom no therapeutic solution exists.

Within RHO gene a few hotspots of mutations have been highlighted such as mutations at codon 23 (Pro23His), codon 135 (Arg135Trp, Arg135Leu, associated with aggressive forms of RP), and codon 347 (Pro347Ala, Pro347Thr, Pro347Leu) for example (Sullivan et al). In a French autosomal dominant rod-cone dystrophies adRP cohort (Audo et al), 16.5% of patient presented a RHO mutation including novel missense mutations (Leu88Pro, Met207Lys, Gln344Pro) as well as previously published mutations (Asn15Ser, Leu131Pro, Arg135Trp, Ser334GlyfsX2, Pro347Leu). In this study Pro347Leu mutation is the most prevalent unlike in American cohorts where Pro23His mutation is the most prevalent, possibly in relation with a fundator effect since many American patients share a common ancestor. However, the general picture is that a wide range of dominant mutations widespread on RHO gene sequence have been associated with RP. The mutational heterogeneity of RHO gene constitutes a major barrier in the development of gene therapy of this dominantly inherited disorder. This feature differs from other genetic diseases where a specific mutation represents/encompasses the vast majority of patients such as in the case of Sickle Cell Disease in which Glu6Val mutation in beta globin HBB gene is predominant.

Current gene therapy strategies are based on a complementation approach, where a functional extra copy of the targeted gene is randomly inserted which provides for the function of the mutated endogenous copy.

Efforts have been made to develop gene therapy methods and models for RP, mostly in mice. As demonstrated in several studies transgene/SiRNA expression can be obtained in the eye/retinal cells by use of viral vectors such as adeno-associated Viral (AAV) vectors (AAV5) (O'Reilly et al; Palfi et al) or Lentiviruses (Takahashi et al). For instance, Palfi et al have demonstrated that a suite of recombinant 2/5 adeno-associated Viral (AAV) vectors could be used to restore RHO expression in the retina of RHO−/− mice.

Because of the dominance of negative mutations into pathologic allele of adRP patients, traditionally used complementation approaches for restoration of the normal function of the gene and the protein can not be implemented. The dominant negative mutation of the pathologic allele must either be corrected or silenced/negated.

To tackle the difficulty associated with dominant negative mutations and mutational heterogeneity O'Reilly et al have combined gene suppression of the endogenous pathologic allele by RNAi delivered by AAV and gene replacement with a siRNA insensitive functional RHO gene in Pro23His mice model.

Homologous gene targeting strategies have been used to knock out endogenous genes (Capecchi M. R., Science, 1989, 244, 1288-1292; Smithies O., Nat Med, 2001, 7, 1083-1086) or knock-in exogenous sequences into the genome. It can as well be used for gene correction, and in principle, for the correction of mutations linked with monogenic diseases. However, gene correction is difficult to achieve clinically, due to the low efficiency of the process (10⁻⁶ to 10⁻⁹ events per transfected cell). In the last decade, several methods have been developed to enhance this yield. For example, chimeraplasty (de Semir D. et al, J Gene Med, 2003, 5, 625-639) and Small Fragment Homologous Replacement (Goncz K. K. et al, Gene Therapy, 2001, 8, 961-965; Sangiuolo F. et al, BMC Med Genet, 2002, 3, 8; Bruscia E. et al., Gene Ther, 2002, 9, 683-685; De Semir D. and Aran J. M., Oligonucleotides, 2003, 13, 261-269) have both been used to try to correct CFTR mutations with various levels of success.

To enhance the efficiency of gene targeting, another strategy to enhance its efficiency is to deliver a DNA double-strand break (DSB) in the targeted locus (FIG. 1), using an enzymatically induced double strand break at or around the locus where recombination is required.

The most accurate way to correct a genetic defect is to use a repair matrix with a non mutated copy of the gene (FIG. 1A), resulting in a reversion of the mutation. However, the efficiency of gene correction decreases as the distance between the mutation and the DSB grows, with a five-fold decrease by 200 bp of distance. Therefore, a given DNA cleaving enzyme can be used to correct with high efficiency only mutations in the vicinity of its DNA target.

An alternative strategy, termed “exon knock-in” is featured in FIG. 1C. In this case, a meganuclease cleaving the gene can be used to knock-in functional exonic sequences upstream of the deleterious mutation. Although this method places the transgene in its regular location, it also results in exon duplication, whose long term impact remains to be seen. In addition, should naturally cis-acting elements be placed in an intron downstream of the cleavage, this alteration to the gene environment could also lead to further unwanted effects such as over or under expression of the altered gene. However, this method has a tremendous advantage in that a single DNA cleaving enzyme could be used to correct any mutation affecting a patient, at least mutations close to or downstream of the enzyme cleavage site.

For this purpose meganucleases have been identified as suitable enzymes to induce the required double-strand break. Meganucleases are by definition sequence-specific endonucleases recognizing large sequences (Thierry, A. and B. Dujon, Nucleic Acids Res., 1992, 20, 5625-5631). They can cleave unique sites in living cells, thereby enhancing gene targeting by 1000-fold or more in the vicinity of the cleavage site (Puchta et al., Nucleic Acids Res., 1993, 21, 5034-5040; Rouet et al., Mol. Cell. Biol., 1994, 14, 8096-8106; Choulika et al., Mol. Cell. Biol., 1995, 15, 1968-1973; Puchta et al., Proc. Natl. Acad. Sci. U.S.A., 1996, 93, 5055-5060; Sargent et al., Mol. Cell. Biol., 1997, 17, 267-277; Cohen-Tannoudji et al., Mol. Cell. Biol., 1998, 18, 1444-1448; Donoho, et al., Mol. Cell. Biol., 1998, 18, 4070-4078; Elliott et al., Mol. Cell. Biol., 1998, 18, 93-101).

Although several hundred natural meganucleases, also referred to as “homing endonucleases” have been identified (Chevalier, B. S. and B. L. Stoddard, Nucleic Acids Res., 2001, 29, 3757-3774), the repertoire of cleavable target sequences is too limited to allow the specific cleavage of a target site in a gene of interest as there is usually no cleavable site in a chosen gene of interest. For example, there is no cleavage site for known naturally occurring I-Cre1 or I-Sce1 meganucleases in human RHO gene.

Theoretically, the making of artificial sequence-specific endonucleases with chosen specificities could alleviate this limit. To overcome this limitation, an approach adopted by a number of workers in this field is the fusion of Zinc-Finger Proteins (ZFPs) with the catalytic domain of FokI, a class IIS restriction endonuclease, so as to make functional sequence-specific endonucleases (Smith et al., Nucleic Acids Res., 1999, 27, 674-681; Bibikova et al., Mol. Cell. Biol., 2001, 21, 289-297; Bibikova et al., Genetics, 2002, 161, 1169-1175; Bibikova et al., Science, 2003, 300, 764; Porteus, M. H. and D. Baltimore, Science, 2003, 300, 763-; Alwin et al., Mol. Ther., 2005, 12, 610-617; Urnov et al., Nature, 2005, 435, 646-651; Porteus, M. H., Mol. Ther., 2006, 13, 438-446). Such ZFP nucleases have been used for the engineering of the IL2RG gene in human lymphoid cells (Urnov et al., Nature, 2005, 435, 646-651).

The binding specificity of Cys2-His2 type Zinc-Finger Proteins, is easy to manipulate because specificity is driven by essentially four residues per zinc finger (Pabo et al., Annu. Rev. Biochem., 2001, 70, 313-340; Jamieson et al., Nat. Rev. Drug Discov., 2003, 2, 361-368). Studies from the Pabo laboratories have resulted in a large repertoire of novel artificial ZFPs, able to bind most G/ANNG/ANNG/ANN sequences (Rebar, E. J. and C. O. Pabo, Science, 1994, 263, 671-673; Kim, J. S. and C. O. Pabo, Proc. Natl. Acad. Sci. U S A, 1998, 95, 2812-2817), Klug (Choo, Y. and A. Klug, Proc. Natl. Acad. Sci. USA, 1994, 91, 11163-11167; Isalan M. and A. Klug, Nat. Biotechnol., 2001, 19, 656-660) and Barbas (Choo, Y. and A. Klug, Proc. Natl. Acad. Sci. USA, 1994, 91, 11163-11167; Isalan M. and A. Klug, Nat. Biotechnol., 2001, 19, 656-660).

Nevertheless, ZFPs have serious limitations, especially for applications requiring a very high level of specificity, such as therapeutic applications. It was shown that FokI nuclease activity in ZFP fusion proteins can act with either one recognition site or with two sites separated by variable distances via a DNA loop (Catto et al., Nucleic Acids Res., 2006, 34, 1711-1720). Thus, the specificities of these ZFP nucleases are degenerate, as illustrated by high levels of toxicity in mammalian cells and Drosophila (Bibikova et al., Genetics, 2002, 161, 1169-1175; Bibikova et al., Science, 2003, 300, 764-.).

To bypass these problems heretofore existing in the art, the inventors have adopted a different approach using engineered meganucleases.

In the wild, meganucleases are essentially represented by homing endonucleases. Homing Endonucleases (HEs) are a widespread family of natural meganucleases including hundreds of proteins families (Chevalier, B. S. and B. L. Stoddard, Nucleic Acids Res., 2001, 29, 3757-3774). These proteins are encoded by mobile genetic elements which propagate by a process called “homing”: the endonuclease cleaves a cognate allele from which the mobile element is absent, thereby stimulating a homologous recombination event that duplicates the mobile DNA into the recipient locus. Given their exceptional cleavage properties in terms of efficacy and specificity, they could represent ideal scaffold to derive novel, highly specific endonucleases.

HEs belong to four major families. The LAGLIDADG family, named after a conserved peptidic motif involved in the catalytic center, is the most widespread and the best characterized group. Seven structures are now available. Whereas most proteins from this family are monomeric and display two LAGLIDADG motifs, a few have only one motif, but dimerize to cleave palindromic or pseudo-palindromic target sequences.

Although the LAGLIDADG peptide is the only conserved region among members of the family, these proteins share a very similar architecture (FIG. 2A). The catalytic core is flanked by two DNA-binding domains with a perfect two-fold symmetry for homodimers such as I-CreI (Chevalier, et al., Nat. Struct. Biol., 2001, 8, 312-316) and I-MsoI (Chevalier et al., J. Mol. Biol., 2003, 329, 253-269) and with a pseudo symmetry for monomers such as I-SceI (Moure et al., J. Mol. Biol., 2003, 334, 685-69, I-DmoI (Silva et al., J. Mol. Biol., 1999, 286, 1123-1136) or I-AniI (Bolduc et al., Genes Dev., 2003, 17, 2875-2888). Both monomers or both domains of monomeric proteins contribute to the catalytic core, organized around divalent cations. Just above the catalytic core, the two LAGLIDADG peptides play also an essential role in the dimerization interface. DNA binding depends on two typical saddle-shaped αββαββα folds, sitting on the DNA major groove. Other domains can be found, for example in inteins such as PI-PfuI (Ichiyanagi et al., J. Mol. Biol., 2000, 300, 889-901) and PI-SceI (Moure et al., Nat. Struct. Biol., 2002, 9, 764-770), which protein splicing domain is also involved in DNA binding.

The making of functional chimeric meganucleases, by fusing the N-terminal I-DmoI domain with an I-CreI monomer (Chevalier et al., Mol. Cell., 2002, 10, 895-905; Epinat et al., Nucleic Acids Res, 2003, 31, 2952-62; International PCT Applications WO 03/078619 and WO 2004/031346) have demonstrated the plasticity of meganucleases.

Different groups have used a semi-rational approach to locally alter the specificity of I-CreI (Seligman et al., Genetics, 1997, 147, 1653-1664; Sussman et al., J. Mol. Biol., 2004, 342, 31-41; International PCT Applications WO 2006/097784 and WO 2006/097853; Arnould et al., J. Mol. Biol., 2006, 355, 443-458; Rosen et al., Nucleic Acids Res., 2006, 34, 4791-4800; Smith et al., Nucleic Acids Res., 2006, 34, e149), I-SceI (Doyon et al., J. Am. Chem. Soc., 2006, 128, 2477-2484), PI-SceI (Gimble et al., J. Mol. Biol., 2003, 334, 993-1008) and I-MsoI (Ashworth et al., Nature, 2006, 441, 656-659).

In addition, hundreds of I-CreI derivatives with locally altered specificity were engineered by combining the semi-rational approach and High Throughput Screening:
Residues Q44, R68 and R70 or Q44, R68, D75 and 177 of I-CreI were mutagenized and a collection of variants with altered specificity at positions ±3 to 5 of the DNA target (5NNN DNA target) were identified by screening (International PCT Applications WO 2006/097784 and WO 2006/097853; Arnould et al., J. Mol. Biol., 2006, 355, 443-458; Smith et al., Nucleic Acids Res., 2006, 34, e149).
Residues K28, N30 and Q38 or N30, Y33, and Q38 or K28, Y33, Q38 and S40 of I-CreI were mutagenized and a collection of variants with altered specificity at positions ±8 to 10 of the DNA target (10NNN DNA target) were identified by screening (Smith et al., Nucleic Acids Res., 2006, 34, e149; International PCT Applications WO 2007/060495 and WO 2007/049156).

Two different variants were combined and assembled in a functional heterodimeric endonuclease able to cleave a chimeric target resulting from the fusion of a different half of each variant DNA target sequence (Arnould et al., precited; International PCT Applications WO 2006/097854 and WO 2007/034262), as illustrated on FIG. 2B. Interestingly, the novel proteins had kept proper folding and stability, high activity, and a narrow specificity.

Furthermore, residues 28 to 40 and 44 to 77 of I-CreI were shown to form two separable functional subdomains, able to bind distinct parts of a homing endonuclease half-site (Smith et al. Nucleic Acids Res., 2006, 34, e149; International PCT Applications WO 2007/049095 and WO 2007/057781).

The combination of mutations from the two subdomains of I-CreI within the same monomer allowed the design of novel chimeric molecules (homodimers) able to cleave a palindromic combined DNA target sequence comprising the nucleotides at positions ±3 to 5 and ±8 to 10 which are bound by each subdomain (Smith et al., Nucleic Acids Res., 2006, 34, e149; International PCT Applications WO 2007/060495 and WO 2007/049156), as illustrated on FIG. 2C.

The combination of the two former steps allows a larger combinatorial approach, involving four different subdomains. The different subdomains can be modified separately and combined to obtain an entirely redesigned meganuclease variant (heterodimer or single-chain molecule) with chosen specificity, as illustrated on FIG. 2D. In a first step, couples of novel meganucleases are combined in new molecules (“half-meganucleases”) cleaving palindromic targets derived from the target one wants to cleave. Then, the combination of such “half-meganuclease” can result in a heterodimeric species cleaving the target of interest. The assembly of four sets of mutations into heterodimeric endonucleases cleaving a model target sequence or a sequence from different genes has been described in the following patent applications: XPC gene (WO2007093918), RAG gene (WO2008010093), HPRT gene (WO2008059382), beta-2 microglobulin gene (WO2008102274), Rosa26 gene (WO2008152523), Human hemoglobin beta gene (WO2009013622) and Human Interleukin-2 receptor gamma chain (WO2009019614).

These variants can be used to cleave genuine chromosomal sequences and have paved the way for novel perspectives in several fields, including gene therapy.

However, even though the base-pairs ±1 and ±2 do not display any contact with the protein, it has been shown that these positions are not devoid of content information (Chevalier et al., J. Mol. Biol., 2003, 329, 253-269), especially for the base-pair ±1 and could be a source of additional substrate specificity (Argast et al., J. Mol. Biol., 1998, 280, 345-353; Jurica et al., Mol. Cell., 1998, 2, 469-476; Chevalier, B. S. and B. L. Stoddard, Nucleic Acids Res., 2001, 29, 3757-3774). In vitro selection of cleavable I-CreI target (Argast et al., precited) randomly mutagenized, revealed the importance of these four base-pairs on protein binding and cleavage activity. It has been suggested that the network of ordered water molecules found in the active site was important for positioning the DNA target (Chevalier et al., Biochemistry, 2004, 43, 14015-14026). In addition, the extensive conformational changes that appear in this region upon I-CreI binding suggest that the four central nucleotides could contribute to the substrate specificity, possibly by sequence dependent conformational preferences (Chevalier et al., 2003, precited). Unexpectedly the inventors have also found active new endonucleases that cleave targets containing changes in these four central nucleotides, which are G₋₂T₋₁A₊₁C₊₂ in the wildtype palindromic I-CreI target C1221 (SEQ ID NO 2).

### SUMMARY OF THE INVENTION

Therefore, in the present invention, endonucleases variants could be used to induce a double strand break in the Human Rhodopsin (RHO) gene and for genome therapy of RP disease and also to allow further experimental study of this important disease in cellular or other types of model systems.

Because the adRP disease involves several genes including RHO, resulting in the expression of aberrant proteins with dominant effects, a traditionally complementation approach to restore the normal function of the gene cannot be implemented; therefore, in the present invention engineered meganucleases has been designed to meet at least one of the following genome therapy strategies:
precise gene correction, implying the engineering of a meganuclease targeting a site located in the vicinity of the mutation and the generation of a repair matrix containing the corresponding non mutated allelic sequences. This strategy relies on Homologous Recombination (HR) of enhanced efficiency due to the meganuclease activity (double strand break) (FIG. 1A). In this case the mutation is precisely corrected and therefore erased fully restoring the Wild-Type (WT) protein function and the structure of WT allele.
Exon Knock In (exon KI), this strategy involves the reconstitution of a functional protein by introduction of a synthetic sequence of the WT coding sequence (cds) while preventing the expression of the pathologic mutations by the integration of stop codons and/or poly-A signals at the end of the functional cds. This strategy also relies on Homologous Recombination (HR) of enhanced efficiency due to the meganuclease activity and on the use of a matrix containing the sequence necessary to reconstitute a functional cds (FIG. 1C). This strategy restores the expression of a functional protein but does not restore a fully WT allele. To apply this strategy, targets present in the beginning of RHO gene are preferred (i.e., first exon and first intron) since any pathologic mutation downstream of the target can be silenced. Mutations of the first exon can also be corrected by the introduction of such exon KI.
Gene inactivation by mutagenesis, this strategy is based on the non-homologous End Joining (NHEJ) mechanism that can take place upon DNA cleavage in absence of repair matrix (FIG. 1B). The NHEJ can produce mutagenesis at the site of cleavage which can result in inactivation of the allele. This strategy can be used to target specific mutation or might be used to cleave a sequence present even in WT gene. In the latter case both normal and pathologic alleles might be inactivated but in the case of dominant negative pathology the inactivation of the WT allele (recessive) should not have significant effect/further noxious effect. In contrast the inactivation of the pathologic allele should allow the WT protein to restore at least partially its function. NHEJ associated mutagenesis might result in the generation of early stop codons, frameshift mutations producing aberrant non functional proteins or could trigger mechanisms such as Nonsense-Mediated mRNA Decay. This strategy is particularly well suited for targets presents at the beginning of the RHO gene which could allow to generate stop codons upstream of most if not all (Nonsense-Mediated mRNA Decay) pathologic dominant mutations.

Unexpectedly the inventors have now found active new endonucleases that cleave targets containing changes in these four central nucleotides, which are G₋₂T₋₁A₊₁C₊₂ in the wild-type palindromic I-CreI target C1221 (SEQ ID NO 2). These variants could be used to induce a double strand break in the Human Rhodopsin (RHO) gene and hence allow the replacement and/or alteration of an endogenous RHO allele(s) so as to treat retinitis pigmentosa disease and also to allow further experimental study of this important disease in cellular or other types of model systems.

The above objects highlight certain aspects of the invention. Additional objects, aspects and embodiments of the invention are found in the following detailed description of the invention.

### BRIEF DESCRIPTION OF THE FIGURES

In addition to the preceding features, the invention further comprises other features which will emerge from the description which follows, which refers to examples illustrating the I-CreI meganuclease variants and their uses according to the invention, as well as to the appended drawings. A more complete appreciation of the invention and many of the attendant advantages thereof will be readily obtained as the same becomes better understood by reference to the following Figures in conjunction with the detailed description below.

FIG. 1: Illustration of two different strategies for restoring a functional gene with meganuclease-induced recombination. A. Gene correction. A mutation occurs within the RHO gene. Upon cleavage by a meganuclease and recombination with a repair matrix the deleterious mutation is corrected. B. Gene inactivation by mutagenesis, this strategy being based on the non homologous End Joining (NHEJ) mechanism that can take place upon DNA cleavage in absence of matrix. The NHEJ can produce mutagenesis at the site of cleavage which can result in inactivation of the allele. C. Exonic sequences knock-in. A mutation occurs within the RHO gene. The mutated mRNA transcript is featured below the gene. In the repair matrix, all exons necessary to reconstitute a complete cDNA are fused in frame, with a polyadenylation site to stop transcription in 3′. Introns and exons sequences can be used as homologous regions. Exonic sequences knock-in results into an engineered gene, transcribed into an mRNA able to code for a functional RHO protein.

FIG. 2: Modular structure of homing endonucleases and the combinatorial approach for custom meganucleases design A. Tridimensional structure of the I-CreI homing endonuclease bound to its DNA target. The catalytic core is surrounded by two (αββαββα folds forming a saddle-shaped interaction interface above the DNA major groove. B. Different binding sequences derived from the I-CreI target sequence (top right and bottom left) to obtain heterodimers or single chain fusion molecules cleaving non palindromic chimeric targets (bottom right). C. The identification of smaller independent subunit, i.e., subunit within a single monomer or αββαββα fold (top right and bottom left) would allow for the design of novel chimeric molecules (bottom right), by combination of mutations within a same monomer. Such molecules would cleave palindromic chimeric targets (bottom right). D. The combination of the two former steps would allow a larger combinatorial approach, involving four different subdomains. In a first step, couples of novel meganucleases could be combined in new molecules (“half-meganucleases”) cleaving palindromic targets derived from the target one wants to cleave. Then, the combination of such “half-meganuclease” can result in an heterodimeric species cleaving the target of interest. Thus, the identification of a small number of new cleavers for each subdomain would allow for the design of a very large number of novel endonucleases.

FIG. 3: Rho34 and Rho34 derived targets. The Rho34.1 target sequence (SEQ ID NO: 8) and its derivatives 10TTC_P (SEQ ID NO: 4), 10GTG_P (SEQ ID NO: 5), 5CAC_P (SEQ ID NO: 6) and 5GTA_P ((SEQ ID NO: 7), P stands for Palindromic) are derivatives of C1221, found to be cleaved by previously obtained I-CreI mutants. C1221, 10TTC_P, 10 GTG_P, 5CAC_P and 5GTA_P were first described as 24 bp sequences, but structural data suggest that only the 22 bp are relevant for protein/DNA interaction. Consequently, positions ±12 are indicated in parenthesis. Rho34.1 (SEQ ID NO: 8) is the DNA sequence located in the human RHO gene at position 259-282. Rho34.2 (SEQ ID NO: 9) differs from Rho34.1 at positions −2;−1;+1;+2 where I-CreI cleavage site (GTAC) substitutes the corresponding Rho34.1 sequence. Rho34.3 (SEQ ID NO: 10) is the palindromic sequence derived from the left part of Rho34.2, and Rho34.4 (SEQ ID NO: 12) is the palindromic sequence derived from the right part of Rho34.2. Rho34.5 (SEQ ID NO: 11) is the palindromic sequence derived from the left part of Rho34.1, and Rho34.6 (SEQ ID NO: 13) is the palindromic sequence derived from the right part of Rho34.1.

FIG. 4: Identification of meganucleases cleaving Rho34.1 target. Variants cleaving Rho34.5 (columns) and Rho34.6 (lanes) where co-expressed in Yeast to form heterodimers.

FIG. 5: Activity cleavage in CHO cells of single chain heterodimer SCOH-ro34-b56-D/Rho34.1 (pCLS3176), SCOH-ro34-b56-A/Rho34.1 (pCLS3189), SCOH-ro34-b56-B/Rho34.1 (pCLS3190), SCOH-ro34-b56-C/Rho34.1 (pCLS3191), SCOH-ro34-b11-C/Rho34.1 (pCLS3488), SCOH-ro34-b11-E/Rho34.1(pCLS3489), compared to ISceI (pCLS1090) and SCOH-RAG-CLS (pCLS2222) meganucleases as positive controls. The empty vector control (pCLS1069) has also been tested on each target. Plasmid pCLS1728 contains control RAG1.10.1 target sequence.

FIG. 6: Rho_{—}7 and Rho_{—}7 derived targets. The Rho_{—}7.1 target sequence (SEQ ID NO: 20) and its derivatives. 10CAG_P (SEQ ID NO: 16), 10TGC_P (SEQ ID NO: 17), 5ACC_P (SEQ ID NO: 18) and 5TCT_P ((SEQ ID NO: 19), P stands for Palindromic) are derivatives of C 1221, found to be cleaved by previously obtained I-CreI mutants. C1221, 10 CAG_P, 10TGC_P, 5ACC_P and 5TCT_P were first described as 24 bp sequences, but structural data suggest that only the 22 bp are relevant for protein/DNA interaction. Consequently, positions ±12 are indicated in parenthesis. Rho_{—}7.1 (SEQ ID NO: 20) is the DNA sequence located in the human RHO gene at position 3915-3938. Rho7.2 (SEQ ID NO: 21) differs from Rho_{—}7.1 at positions −2;−1;+1;+2 where I-CreI cleavage site (GTAC) substitutes the corresponding Rho_{—}7.1 sequence. Rho_{—}7.3 (SEQ ID NO: 22) is the palindromic sequence derived from the left part of Rho7.2, and Rho_{—}7.4 (SEQ ID NO: 23) is the palindromic sequence derived from the right part of Rho_{—}7.2. Rho_{—}7.5 (SEQ ID NO: 24) is the palindromic sequence derived from the left part of Rho7.1, and Rho_{—}7.6 (SEQ ID NO: 25) is the palindromic sequence derived from the right part of Rho_{—}7.1.

FIG. 7: Identification of meganucleases cleaving Rho_{—}7.1 target. Variants cleaving Rho_{—}7.5 (lanes) and Rho_{—}7.6 (columns) where co-expressed in Yeast to form heterodimers.

FIG. 8: Activity cleavage in CHO cells of single chain heterodimer SCOH-ro7-b56-C/Rho7.1 (pCLS3482) and SCOH-ro7-b1-C/Rho7.1 (pCLS3491), compared to ISceI (pCLS1090) and SCOH-RAG-CLS (pCLS2222) meganucleases as positive controls. The empty vector control (pCLS1069) has also been tested on each target. Plasmid pCLS1728 contains control RAG1.10.1 target sequence.

FIG. 9: Rho36 and Rho36 derived targets. The Rho36.1 target sequence (SEQ ID NO: 32) and its derivatives. 10GAT_P (SEQ ID NO: 28), 10CCT_P (SEQ ID NO: 30), 5CAC_P (SEQ ID NO: 29) and 5CTG_P ((SEQ ID NO: 31), P stands for Palindromic) are derivatives of C1221 found to be cleaved by previously obtained I-CreI mutants. C1221, 10GAT_P, 10CCT_P, 5CAC_P and 5CTG_P were first described as 24 bp sequences, but structural data suggest that only the 22 bp are relevant for protein/DNA interaction. Consequently, positions ±12 are indicated in parenthesis. Rho36.1 (SEQ ID NO: 32) is the DNA sequence located in the human RHO gene at position 1177-1200. Rho36.2 (SEQ ID NO: 33) differs from Rho36.1 at positions −2;−1;+1;+2 where I-CreI cleavage site (GTAC) substitutes the corresponding Rho36.1 sequence. Rho36.3 (SEQ ID NO: 34) is the palindromic sequence derived from the left part of Rho36.2, and Rho36.4 (SEQ ID NO: 35) is the palindromic sequence derived from the right part of Rho36.2. Rho36.5 (SEQ ID NO: 36) is the palindromic sequence derived from the left part of Rho36.1, and Rho36.6 (SEQ ID NO: 37) is the palindromic sequence derived from the right part of Rho36.1.

FIG. 10: Vector Map of pCLS1072

FIG. 11: Vector Map of pCLS1090

FIG. 12: Vector Map of pCLS2222

FIG. 13: Vector Map of pCLS1853

FIG. 14: Vector Map of pCLS1107

FIG. 15: Vector Map of pCLS 1090

FIG. 16: Vector Map of pCLS1069

FIG. 17: Vector Map of pCLS 1058

FIG. 18: Vector Map of pCLS1055

FIG. 19: Vector Map of pCLS0542

FIG. 20: Vector Map of pCLS 1728

FIG. 21: Rho31 and Rho31 derived targets. The Rho31.1 target sequence (SEQ ID NO: 86) and its derivatives 10AGG_P (SEQ ID NO: 80), 10CCT_P (SEQ ID NO: 81), 5CTT_P (SEQ ID NO: 82) and 5CCA_P (SEQ ID NO: 83), P stands for Palindromic) are derivatives of C1221, found to be cleaved by previously obtained I-CreI mutants. C1221, 10AGG_P, 10CCT_P, 5CTT_P and 5CCA_P were first described as 24 bp sequences, but structural data suggest that only the 22 bp are relevant for protein/DNA interaction. Consequently, positions ±12 are indicated in parenthesis. Rho31.1 (SEQ ID NO: 86) is the DNA sequence located in the region upstream of exon 1 of RHO gene as described in Table IX. Rho31.2 (SEQ ID NO: 87) differs from Rho31.1 at positions −2;−1;+1;+2 where I-CreI cleavage site (GTAC) substitutes the corresponding Rho31.1 sequence. Rho31.3 (SEQ ID NO: 88) is the palindromic sequence derived from the left part of Rho31.2, and Rho31.4 (SEQ ID NO: 89) is the palindromic sequence derived from the right part of Rho31.2. Rho31.5 (SEQ ID NO: 90) is the palindromic sequence derived from the left part of Rho31.1, and Rho31.6 (SEQ ID NO: 91) is the palindromic sequence derived from the right part of Rho31.1.

### DETAILED DESCRIPTION OF THE INVENTION

Unless specifically defined herein below, all technical and scientific terms used herein have the same meaning as commonly understood by a skilled artisan in the fields of gene therapy, biochemistry, genetics, and molecular biology.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will control. Further, the materials, methods, and examples are illustrative only and are not intended to be limiting, unless otherwise specified.

According to a first aspect of the present invention is an I-CreI variant, which has two I-CreI monomers and at least one of the two I-CreI monomers has at least two substitutions, where there is at least one mutation in each of the two functional subdomains of the LAGLIDADG core domain situated from positions 26 to 40 and 44 to 77 of I-CreI, respectively, and said variant cleaves a DNA target sequence from the Rhodopsin gene (RHO). Within this embodiment, the I-CreI variant is obtained by a method comprising at least the steps of:

(a) constructing a first series of I-CreI variants having at least one substitution in a first functional subdomain of the LAGLIDADG core domain situated from positions 26 to 40 of I-CreI,

(b) constructing a second series of I-CreI variants having at least one substitution in a second functional subdomain of the LAGLIDADG core domain situated from positions 44 to 77 of I-CreI,

(c) selecting and/or screening the variants from the first series of step (a) which are able to cleave a mutant I-CreI site wherein at least one of (i) the nucleotide triplet in positions −10 to −8 of the I-CreI site has been replaced with the nucleotide triplet which is present in positions −10 to −8 of said DNA target sequence from RHO and (ii) the nucleotide triplet in positions +8 to +10 has been replaced with the reverse complementary sequence of the nucleotide triplet which is present in position −10 to −8 of said DNA target sequence from RHO,

(d) selecting and/or screening the variants from the second series of step (b) which are able to cleave a mutant I-CreI site wherein at least one of (i) the nucleotide triplet in positions −5 to −3 of the I-CreI site has been replaced with the nucleotide triplet which is present in positions −5 to −3 of said DNA target sequence from RHO and (ii) the nucleotide triplet in positions +3 to +5 has been replaced with the reverse complementary sequence of the nucleotide triplet which is present in position −5 to −3 of said DNA target sequence from RHO,

(e) selecting and/or screening the variants from the first series of step (a) which are able to cleave a mutant I-CreI site wherein at least one of (i) the nucleotide triplet in positions +8 to +10 of the I-CreI site has been replaced with the nucleotide triplet which is present in positions +8 to +10 of said DNA target sequence from RHO and (ii) the nucleotide triplet in positions −10 to −8 has been replaced with the reverse complementary sequence of the nucleotide triplet which is present in position +8 to +10 of said DNA target sequence from RHO,

(f) selecting and/or screening the variants from the second series of step (b) which are able to cleave a mutant I-CreI site wherein at least one of (i) the nucleotide triplet in positions +3 to +5 of the I-CreI site has been replaced with the nucleotide triplet which is present in positions +3 to +5 of said DNA target sequence from RHO and (ii) the nucleotide triplet in positions −5 to −3 has been replaced with the reverse complementary sequence of the nucleotide triplet which is present in position +3 to +5 of said DNA target sequence from RHO,

(g) combining in a single variant, the mutation(s) in positions 26 to 40 and 44 to 77 of two variants from step (c) and step (d), to obtain a novel homodimeric I-CreI variant which cleaves a sequence wherein (i) the nucleotide triplet in positions −10 to −8 is identical to the nucleotide triplet which is present in positions −10 to −8 of said DNA target sequence from RHO, (ii) the nucleotide triplet in positions +8 to +10 is identical to the reverse complementary sequence of the nucleotide triplet which is present in positions −10 to −8 of said DNA target sequence from RHO, (iii) the nucleotide triplet in positions −5 to −3 is identical to the nucleotide triplet which is present in positions −5 to −3 of said DNA target sequence from RHO and (iv) the nucleotide triplet in positions +3 to +5 is identical to the reverse complementary sequence of the nucleotide triplet which is present in positions −5 to −3 of said DNA target sequence from RHO, and/or

(h) combining in a single variant, the mutation(s) in positions 26 to 40 and 44 to 77 of two variants from step (e) and step (f), to obtain a novel homodimeric I-CreI variant which cleaves a sequence wherein (i) the nucleotide triplet in positions +8 to +10 of the I-CreI site has been replaced with the nucleotide triplet which is present in positions +8 to +10 of said DNA target sequence from RHO and (ii) the nucleotide triplet in positions −10 to −8 is identical to the reverse complementary sequence of the nucleotide triplet in positions +8 to +10 of said DNA target sequence from RHO, (iii) the nucleotide triplet in positions +3 to +5 is identical to the nucleotide triplet which is present in positions +3 to +5 of said DNA target sequence from RHO, (iv) the nucleotide triplet in positions −5 to −3 is identical to the reverse complementary sequence of the nucleotide triplet which is present in positions +3 to +5 of said DNA target sequence from RHO,

(i) combining the variants obtained in steps (g) and (h) to form heterodimers, and

(j) selecting and/or screening the heterodimers from step (i) which cleave said DNA target sequence from RHO.

In the present patent application the terms meganuclease (s) and variant (s) and variant meganuclease (s) will be used interchangeably herein.

One of the step(s) (c), (d), (e), (f), (g), (h) or (i) may be omitted. For example, if step (c) is omitted, step (d) is performed with a mutant I-CreI target wherein both nucleotide triplets at positions −10 to −8 and −5 to −3 have been replaced with the nucleotide triplets which are present at positions −10 to −8 and −5 to −3, respectively of said genomic target, and the nucleotide triplets at positions +3 to +5 and +8 to +10 have been replaced with the reverse complementary sequence of the nucleotide triplets which are present at positions −5 to −3 and −10 to −8, respectively of said genomic target.

The (intramolecular) combination of mutations in steps (g) and (h) may be performed by amplifying overlapping fragments comprising each of the two subdomains, according to well-known overlapping PCR techniques.

The (intermolecular) combination of the variants in step (i) is performed by co-expressing one variant from step (g) with one variant from step (h), so as to allow the formation of heterodimers. For example, host cells may be modified by one or two recombinant expression vector(s) encoding said variant(s). The cells are then cultured under conditions allowing the expression of the variant(s), so that heterodimers are formed in the host cells, as described previously in the International PCT Application WO 2006/097854 and Arnould et al., J. Mol. Biol., 2006, 355, 443-458.

The selection and/or screening in steps (c), (d), (e), (f), and/or (j) may be performed by measuring the cleavage activity of the variant according to the invention by any well-known, in vitro or in vivo cleavage assay, such as those described in the International PCT Application WO 2004/067736; Epinat et al., Nucleic Acids Res., 2003, 31, 2952-2962; Chames et al., Nucleic Acids Res., 2005, 33, e178; Arnould et al., J. Mol. Biol., 2006, 355, 443-458, and Arnould et al., J. Mol. Biol., 2007, 371, 49-65. For example, the cleavage activity of the variant of the invention may be measured by a direct repeat recombination assay, in yeast or mammalian cells, using a reporter vector. The reporter vector comprises two truncated, non-functional copies of a reporter gene (direct repeats) and the genomic (non-palindromic) DNA target sequence within the intervening sequence, cloned in yeast or in a mammalian expression vector. Usually, the genomic DNA target sequence comprises one different half of each (palindromic or pseudo-palindromic) parent homodimeric I-CreI meganuclease target sequence. Expression of the heterodimeric variant results in a functional endonuclease which is able to cleave the genomic DNA target sequence. This cleavage induces homologous recombination between the direct repeats, resulting in a functional reporter gene, whose expression can be monitored by an appropriate assay. The cleavage activity of the variant against the genomic DNA target may be compared to wild type I-CreI or I-SceI activity against their natural target.

According to another advantageous embodiment of said method, steps (c), (d), (e), (f) and/or (j) are performed in vivo, under conditions where the double-strand break in the mutated DNA target sequence which is generated by said variant leads to the activation of a positive selection marker or a reporter gene, or the inactivation of a negative selection marker or a reporter gene, by recombination-mediated repair of said DNA double-strand break.

Furthermore, the homodimeric combined variants obtained in step (g) or (h) are advantageously submitted to a selection/screening step to identify those which are able to cleave a pseudo-palindromic sequence wherein at least the nucleotides at positions −11 to −3 (combined variant of step (g)) or +3 to +11 (combined variant of step (h)) are identical to the nucleotides which are present at positions −11 to −3 (combined variant of step (g)) or +3 to +11 (combined variant of step (h)) of said genomic target, and the nucleotides at positions +3 to +11 (combined variant of step (g)) or −11 to −3 (combined variant of step (h)) are identical to the reverse complementary sequence of the nucleotides which are present at positions −11 to −3 (combined variant of step (g)) or +3 to +11 (combined variant of step (h)) of said genomic target.

Preferably, the set of combined variants of step (g) or step (h) (or both sets) undergoes an additional selection/screening step to identify the variants which are able to cleave a pseudo-palindromic sequence wherein:

(1) the nucleotides at positions −11 to −3 (combined variant of step (g)) or +3 to +11 (combined variant of step (h)) are identical to the nucleotides which are present at positions −11 to −3 (combined variant of step (g)) or +3 to +11 (combined variant of step h)) of said genomic target, and

(2) the nucleotides at positions +3 to +11 (combined variant of step (g)) or −11 to −3 (combined variant of step (h)) are identical to the reverse complementary sequence of the nucleotides which are present at positions −11 to −3 (combined variant of step (g)) or +3 to +11 (combined variant of step (h)) of said genomic target.

This additional screening step increases the probability of isolating heterodimers which are able to cleave the genomic target of interest (step (k)).

Steps (a), (b), (g), (h) and (i) may further comprise the introduction of additional mutations at other positions contacting the DNA target sequence or interacting directly or indirectly with said DNA target, at positions which improve the binding and/or cleavage properties of the variants, or at positions which either prevent or impair the formation of functional homodimers or favor the formation of the heterodimer, as defined above.

The additional mutations may be introduced by site-directed mutagenesis and/or random mutagenesis on a variant or on a pool of variants, according to standard mutagenesis methods which are well-known in the art, for example by using PCR.

In particular, random mutations may be introduced into the whole variant or in a part of the variant to improve the binding and/or cleavage properties of the variants towards the DNA target from the gene of interest.

Site-directed mutagenesis at positions which improve the binding and/or cleavage properties of the variants, for example at positions 19, 54, 80, 87, 105 and/or 132, may also be combined with random-mutagenesis. The mutagenesis may be performed by generating random/site-directed mutagenesis libraries on a pool of variants, according to standard mutagenesis methods which are well-known in the art. Site-directed mutagenesis may be advantageously performed by amplifying overlapping fragments comprising the mutated position(s), as defined above, according to well-known overlapping PCR techniques. In addition, multiple site-directed mutagenesis, may advantageously be performed on a variant or on a pool of variants.

Preferably, the mutagenesis is performed on one monomer of the heterodimer formed in step (i) or step (j), advantageously on a pool of monomers, preferably on both monomers of the heterodimer of step (i) or (j).

Possibly or not, at least two rounds of selection/screening are performed according to the process illustrated Arnould et al., J. Mol. Biol., 2007, 371, 49-65. In the first round, one of the monomers of the heterodimer is mutagenised, co-expressed with the other monomer to form heterodimers, and the improved monomers Y⁺ are selected against the target from the gene of interest. In the second round, the other monomer (monomer X) is mutagenised, co-expressed with the improved monomers Y⁺ to form heterodimers, and selected against the target from the gene of interest to obtain meganucleases (X⁺ Y⁺) with improved activity. The mutagenesis may be random-mutagenesis or site-directed mutagenesis on a monomer or on a pool of monomers, as indicated above. Both types of mutagenesis are advantageously combined. Additional rounds of selection/screening on one or both monomers may be performed to improve the cleavage activity of the variant.

Preferably the variant may be obtained by a method comprising the additional steps of:

(k) selecting heterodimers from step (j) and constructing a third series of variants having at least one substitution in at least one of the monomers in said selected heterodimers,

(l) combining said third series variants of step (k) and screening the resulting heterodimers for altered cleavage activity against said DNA target from RHO.

Preferably in step (k) at least one substitution is introduced by site directed mutagenesis in a DNA molecule encoding said third series of variants, and/or by random mutagenesis in a DNA molecule encoding said third series of variants.

Preferably steps (k) and (l) are repeated at least two times and wherein the heterodimers selected in step (k) of each further iteration are selected from heterodimers screened in step (l) of the previous iteration which showed altered cleavage activity against said DNA target from RHO.

Target sequences can be chosen in any region of RHO, but in particular are best positioned as close as possible to the locations of known disease causing mutations wherein the variant is for use in a gene repair therapy using a DNA repair matrix. Alternatively the target sequence may be chosen at the beginning of RHO if the variant is for use in an “exon knock-in” method or if the purpose is to induce gene/allele inactivation by NHEJ related mutagenesis, by the creation of early stop codon, frameshift producing aberrant non functional proteins or even Nonsense-Mediated mRNA Decay.

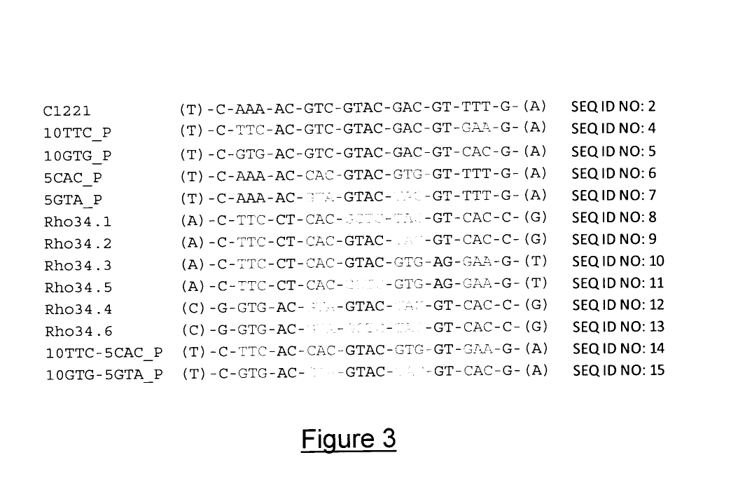

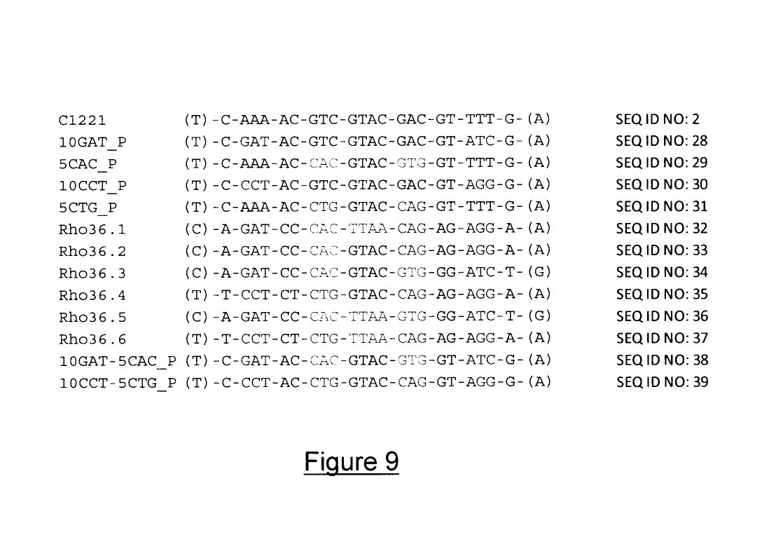

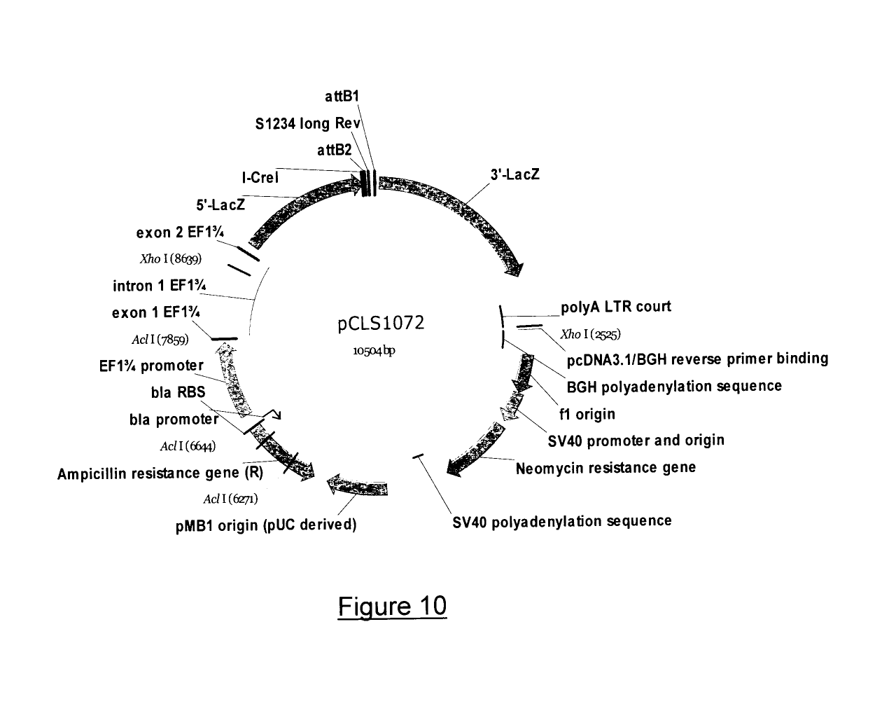

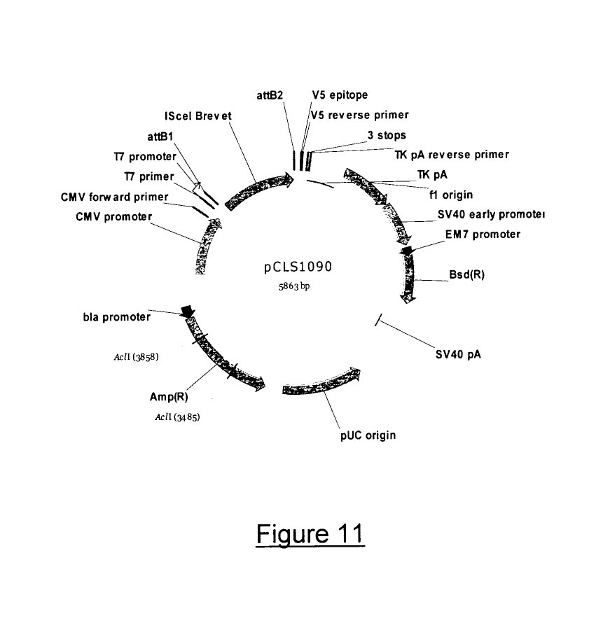

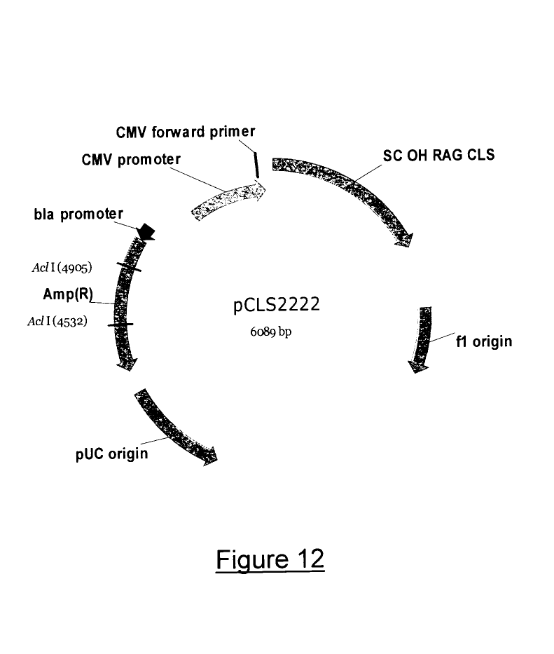

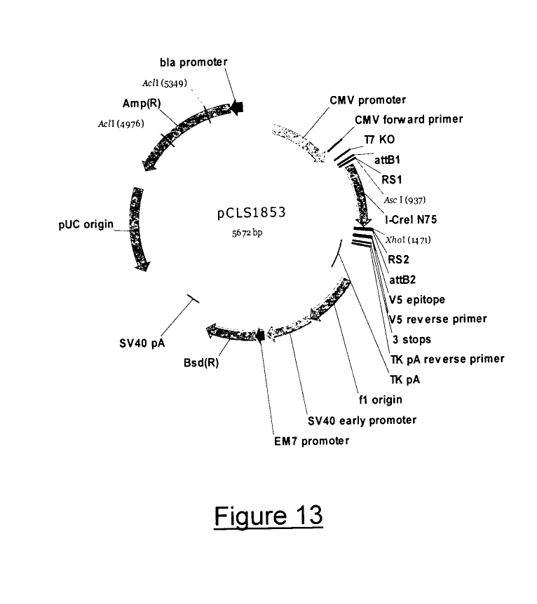

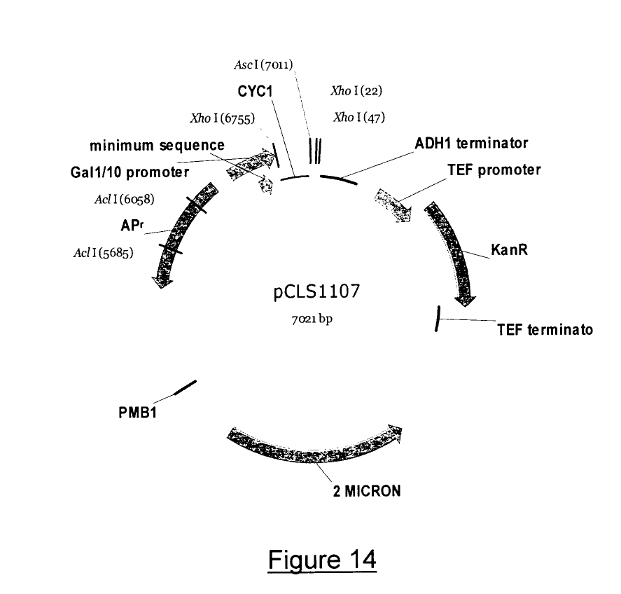

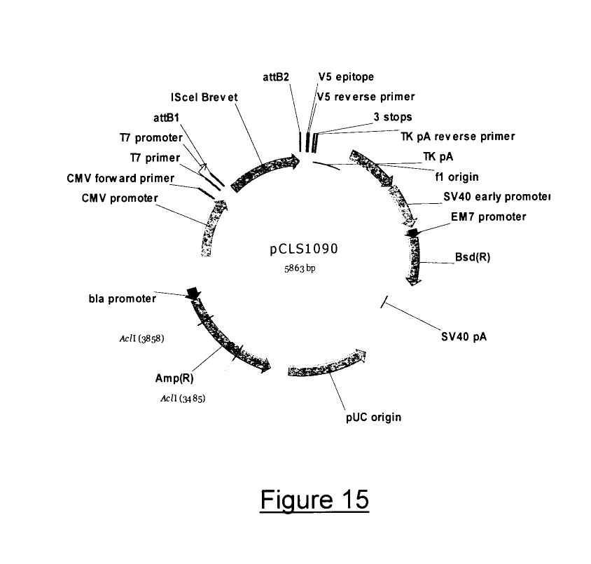

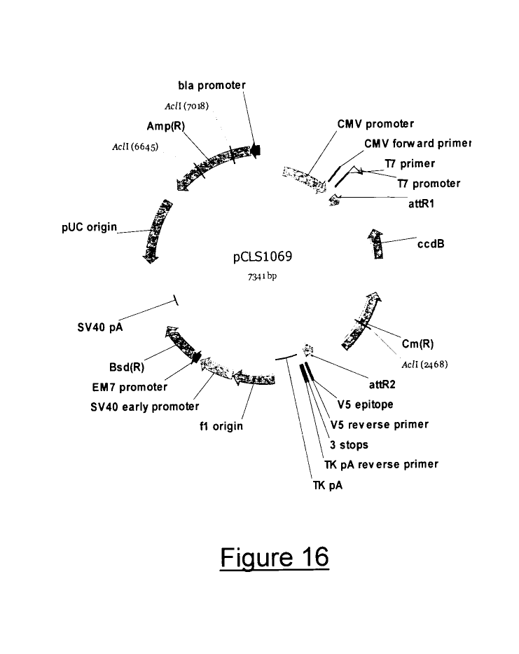

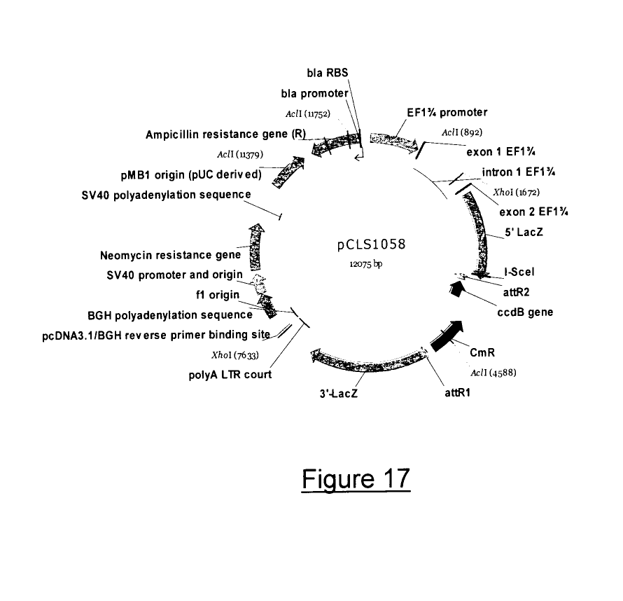

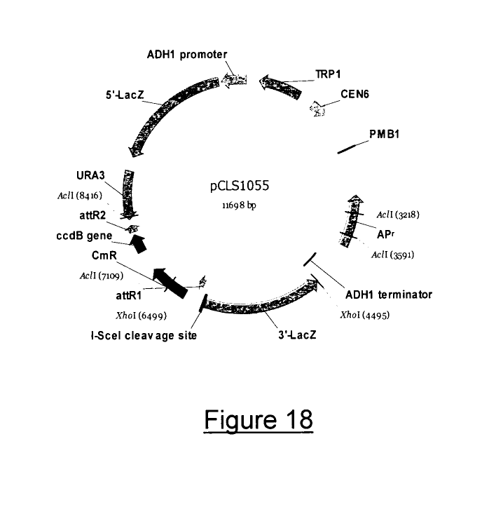

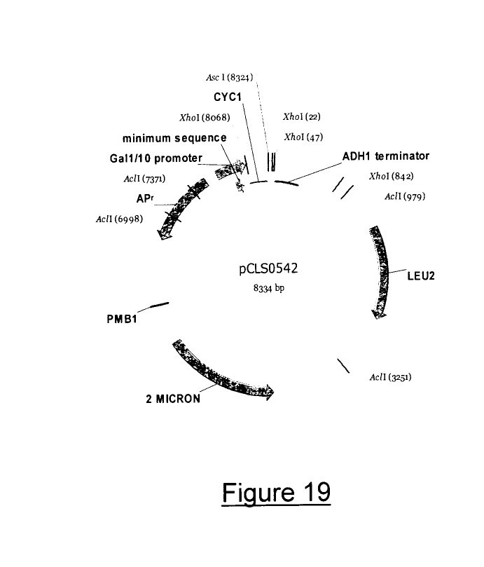

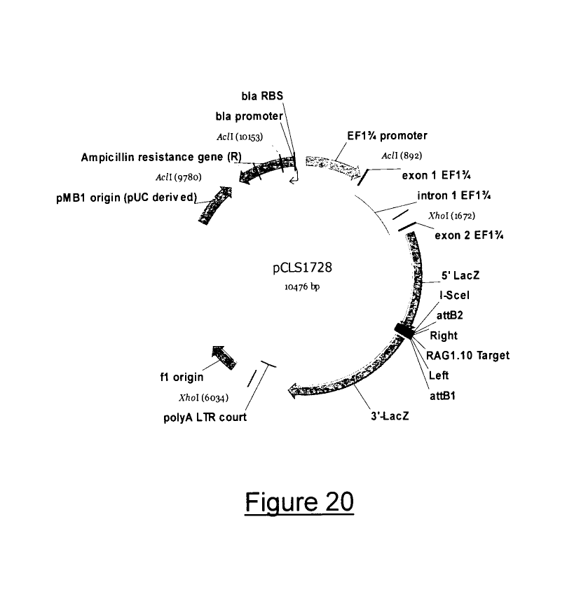

I-CreI variants to these targets were created using a combinatorial approach, to entirely redesign the DNA binding domain of the I-CreI protein and thereby engineer novel meganucleases with fully engineered specificity for the desired RHO target. Some of the DNA targets identified by the inventors to validate there invention are given in FIGS. 3, 6 and 9.

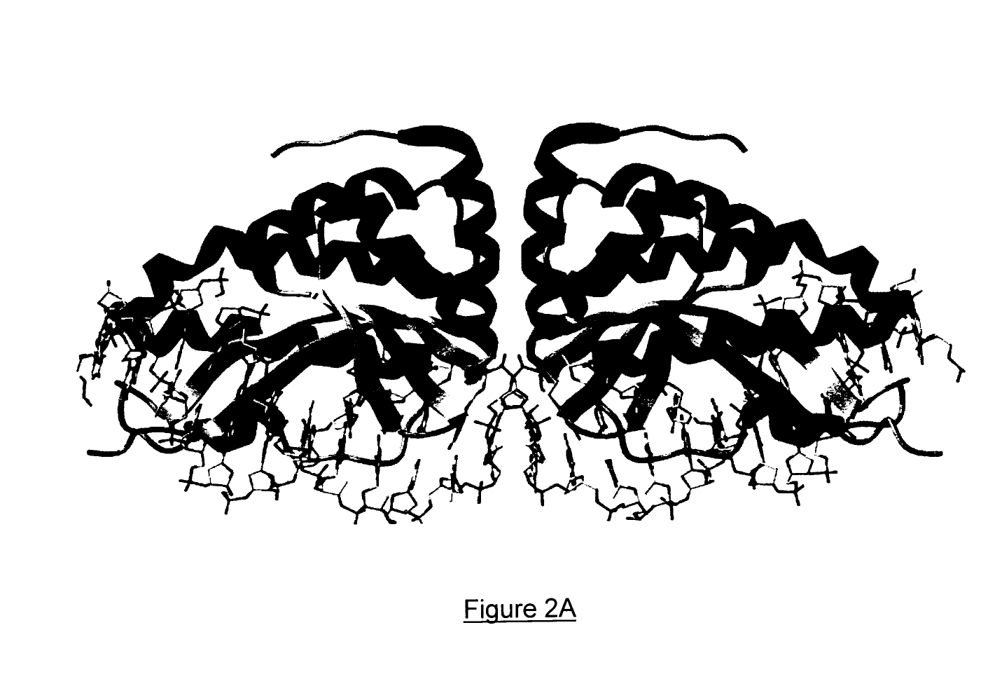

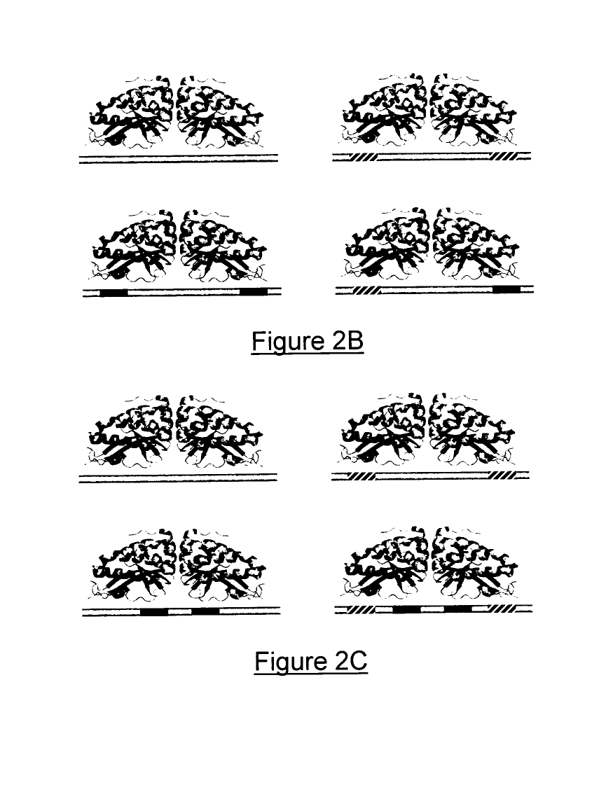

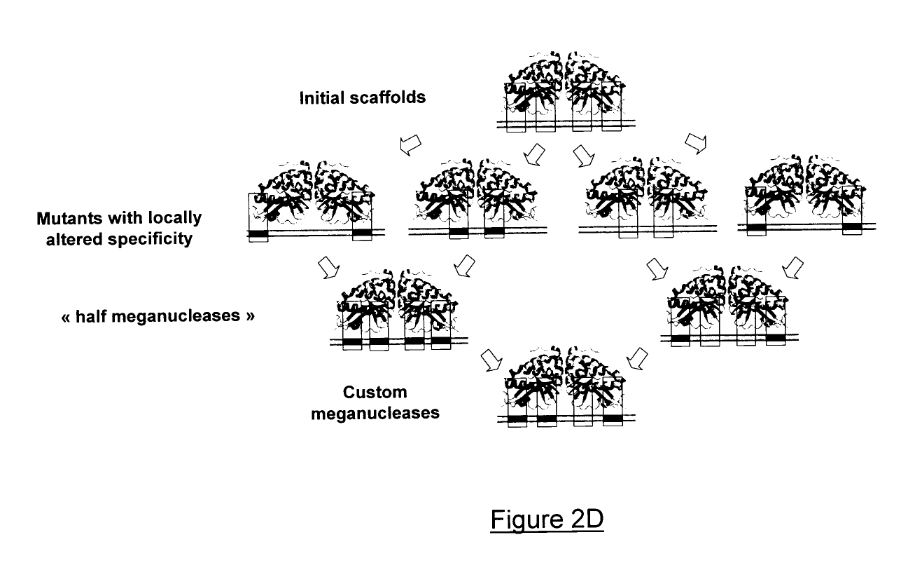

The combinatorial approach, as illustrated in FIG. 2D was used to entirely redesign the DNA binding domain of the I-CreI protein and thereby engineer novel meganucleases with fully engineered specificity.

In particular the heterodimer of step (i) may comprise monomers obtained in steps (g) and (h), with the same DNA target recognition and cleavage activity properties.

Alternatively the heterodimer of step (i) may comprise monomers obtained in steps (g) and (h), with different DNA target recognition and cleavage activity properties.

In particular the first series of I-CreI variants of step (a) are derived from a first parent meganuclease.

In particular the second series of variants of step (b) are derived from a second parent meganuclease.

In particular the first and second parent meganucleases are identical.

Alternatively the first and second parent meganucleases are different.

In particular the variant may be obtained by a method comprising the additional steps of:

(k) selecting heterodimers from step (j) and constructing a third series of variants having at least one substitution in at least one of the monomers of said selected heterodimers,

(l) combining said third series variants of step (k) and screening the resulting heterodimers for enhanced cleavage activity against said DNA target from RHO.

In a preferred embodiment of said variant, said substitution(s) in the subdomain situated from positions 44 to 77 of I-CreI are at positions 44, 68, 70, 75 and/or 77.

In another preferred embodiment of said variant, said substitution(s) in the subdomain situated from positions 28 to 40 of I-CreI are at positions 28, 30, 32, 33, 38 and/or 40.

In another preferred embodiment of said variant, it comprises one or more mutations in I-CreI monomer(s) at positions of other amino acid residues that contact the DNA target sequence or interact with the DNA backbone or with the nucleotide bases, directly or via a water molecule; these residues are well-known in the art (Jurica et al., Molecular Cell., 1998, 2, 469-476; Chevalier et al., J. Mol. Biol., 2003, 329, 253-269). In particular, additional substitutions may be introduced at positions contacting the phosphate backbone, for example in the final C-terminal loop (positions 137 to 143; Prieto et al., Nucleic Acids Res., Epub 22 Apr. 2007).

Preferably said residues are involved in binding and cleavage of said DNA cleavage site.

More preferably, said residues are at positions 138, 139, 142 or 143 of I-CreI. Two residues may be mutated in one variant provided that each mutation is in a different pair of residues chosen from the pair of residues at positions 138 and 139 and the pair of residues at positions 142 and 143. The mutations which are introduced modify the interaction(s) of said amino acid(s) of the final C-terminal loop with the phosphate backbone of the I-CreI site. Preferably, the residue at position 138 or 139 is substituted by a hydrophobic amino acid to avoid the formation of hydrogen bonds with the phosphate backbone of the DNA cleavage site. For example, the residue at position 138 is substituted by an alanine or the residue at position 139 is substituted by a methionine. The residue at position 142 or 143 is advantageously substituted by a small amino acid, for example a glycine, to decrease the size of the side chains of these amino acid residues.

More preferably, said substitution in the final C-terminal loop modify the specificity of the variant towards the nucleotide at positions ±1 to 2, ±6 to 7 and/or ±11 to 12 of the I-CreI site.

In another preferred embodiment of said variant, it comprises one or more additional mutations that improve the binding and/or the cleavage properties of the variant towards the DNA target sequence from the RHO gene. The additional residues which are mutated may be on the entire I-CreI sequence, and in particular in the C-terminal half of I-CreI (positions 80 to 163). Both I-CreI monomers are advantageously mutated; the mutation(s) in each monomer may be identical or different. For example, the variant comprises one or more additional substitutions at positions: 2, 19, 43, 80 and 81. Said substitutions are advantageously selected from the group consisting of: N2S, G19S, F43L, E80K and I81T. More preferably, the variant comprises at least one substitution selected from the group consisting of: N2S, G19S, F43L, E80K and I81T. The variant may also comprise additional residues at the C-terminus. For example a glycine (G) and/or a proline (P) residue may be inserted at positions 164 and 165 of I-CreI, respectively.

According to a preferred embodiment, said additional mutation in said variant further impairs the formation of a functional homodimer. More preferably, said mutation is the G19S mutation. The G19S mutation is advantageously introduced in one of the two monomers of a heterodimeric I-CreI variant, so as to obtain a meganuclease having enhanced cleavage activity and enhanced cleavage specificity. In addition, to enhance the cleavage specificity further, the other monomer may carry a distinct mutation that impairs the formation of a functional homodimer or favors the formation of the heterodimer.

In another preferred embodiment of said variant, said substitutions are replacement of the initial amino acids with amino acids selected from the group consisting of: A, D, E, G, H, K, N, P, Q, R, S, T, Y, C, V, L, M, F, I and W.

In particular the variant is selected from the group consisting of SEQ ID NO: 40 to 65, SEQ ID NO: 92 to 103 and SEQ ID NO: 105 to 116.

The variant of the invention may be derived from the wild-type I-CreI (SEQ ID NO: 1). preferred are where the variant of the invention is derived from an I-CreI scaffold protein having at least 85% identity, at least 90% identity, at least 95% identity, at least 96% identity, at least 97% identity, at least 98% identity, and at least 99% identity with SEQ ID NO: 1 such as the scaffold called I-CreI N75 (167 amino acids; SEQ ID NO: 3) having the insertion of an alanine at position 2, and the insertion of AAD at the C-terminus (positions 164 to 166) of the I-CreI sequence. In the present patent application all the I-CreI variants described comprise an additional Alanine after the first Methionine of the wild type I-CreI sequence (SEQ ID NO: 1). These variants also comprise two additional Alanine residues and an Aspartic Acid residue after the final Proline of the wild type I-CreI sequence. These additional residues do not affect the properties of the enzyme and to avoid confusion these additional residues do not affect the numeration of the residues in I-CreI or a variant referred in the present patent application, as these references exclusively refer to residues of the wild type I-CreI enzyme (SEQ ID NO: 1) as present in the variant, so for instance residue 2 of 1-CreI is in fact residue 3 of a variant which comprises an additional Alanine after the first Methionine.

In addition, the variants of the invention may include one or more residues inserted at the NH₂ terminus and/or COOH terminus of the sequence. For example, a tag (epitope or polyhistidine sequence) is introduced at the NH₂ terminus and/or COOH terminus; said tag is useful for the detection and/or the purification of said variant. The variant may also comprise a nuclear localization signal (NLS); said NLS is useful for the importation of said variant into the cell nucleus. The NLS may be inserted just after the first methionine of the variant or just after an N-terminal tag. As a non limited example, it has been reported that C-terminal part of RHO gene is important for transport of Rhodopsin to the membrane; in this case, a locus such as Rho_{—}7, as described in more details below, might be used to generate mutants deficient in C-term part of Rhodopsin, thereby affected in Rhodopsin transport to the membrane.

The variant according to the present invention may be a homodimer which is able to cleave a palindromic or pseudo-palindromic DNA target sequence.

Alternatively, said variant is a heterodimer, resulting from the association of a first and a second monomer having different substitutions at positions 28 to 40 and 44 to 77 of I-CreI, said heterodimer being able to cleave a non-palindromic DNA target sequence from the RHO gene.

In particular said heterodimer variant is composed by one of the possible associations between variants from the group consisting of SEQ ID NO: 40 to 52, SEQ ID NO: 53 to 65, SEQ ID NO: 92 to 103 and SEQ ID NO: 105 to 116 respectively.

The DNA target sequences are situated in the RHO ORF and these sequences cover all the RHO ORF. In particular said DNA target sequences for the variant of the present invention are selected from the group consisting of the SEQ ID NO: 8 to 13, 20 to 25, 32 to 37 and 86 to 91.

The sequence of each I-CreI variant is defined by the mutated residues at the indicated positions. The positions are indicated by reference to I-CreI sequence (SEQ ID NO: 1); I-CreI has N, S, Y, Q, S, Q, R, R, D, I and E at positions 30, 32, 33, 38, 40, 44, 68, 70, 75, 77 and 80 respectively.

Each monomer (first monomer and second monomer) of the heterodimeric variant according to the present invention may also be named with a letter code, after the eleven residues at positions 28, 30, 32, 33, 38, 40, 44, 68 and 70, 75 and 77 and the additional residues which are mutated, as indicated above. For example, 32T33C38H44V68Y70S75R77V100R (SEQ ID NO: 40).

The heterodimeric variant as defined above may have only the amino acid substitutions as indicated above. In this case, the positions which are not indicated are not mutated and thus correspond to the wild-type I-CreI (SEQ ID NO: 1).

The invention encompasses I-CreI variants having at least 85% identity, preferably at least 90% identity, more preferably at least 95% (96%, 97%, 98%, 99%) identity with the sequences as defined above, said variant being able to cleave a DNA target from the RHO gene.

The heterodimeric variant is advantageously an obligate heterodimer variant having at least one pair of mutations corresponding to residues of the first and the second monomers which make an intermolecular interaction between the two I-CreI monomers, wherein the first mutation of said pair(s) is in the first monomer and the second mutation of said pair(s) is in the second monomer and said pair(s) of mutations prevent the formation of functional homodimers from each monomer and allow the formation of a functional heterodimer, able to cleave the genomic DNA target from the RHO gene.

To form an obligate heterodimer, the monomers have advantageously at least one of the following pairs of mutations, respectively for the first monomer and the second monomer:

a) the substitution of the glutamic acid at position 8 with a basic amino acid, preferably an arginine (first monomer) and the substitution of the lysine at position 7 with an acidic amino acid, preferably a glutamic acid (second monomer); the first monomer may further comprise the substitution of at least one of the lysine residues at positions 7 and 96, by an arginine,

b) the substitution of the glutamic acid at position 61 with a basic amino acid, preferably an arginine (first monomer) and the substitution of the lysine at position 96 with an acidic amino acid, preferably a glutamic acid (second monomer); the first monomer may further comprise the substitution of at least one of the lysine residues at positions 7 and 96, by an arginine,

c) the substitution of the leucine at position 97 with an aromatic amino acid, preferably a phenylalanine (first monomer) and the substitution of the phenylalanine at position 54 with a small amino acid, preferably a glycine (second monomer); the first monomer may further comprise the substitution of the phenylalanine at position 54 by a tryptophane and the second monomer may further comprise the substitution of the leucine at position 58 or lysine at position 57, by a methionine, and

d) the substitution of the aspartic acid at position 137 with a basic amino acid, preferably an arginine (first monomer) and the substitution of the arginine at position 51 with an acidic amino acid, preferably a glutamic acid (second monomer).

For example, the first monomer may have the mutation D137R and the second monomer, the mutation R51D. The obligate heterodimer meganuclease comprises advantageously, at least two pairs of mutations as defined in a), b), c) or d), above; one of the pairs of mutation is advantageously as defined in c) or d). Preferably, one monomer comprises the substitution of the lysine residues at positions 7 and 96 by an acidic amino acid (aspartic acid (D) or glutamic acid (E)), preferably a glutamic acid (K7E and K96E) and the other monomer comprises the substitution of the glutamic acid residues at positions 8 and 61 by a basic amino acid (arginine (R) or lysine (K); for example, E8K and E61R). More preferably, the obligate heterodimer meganuclease, comprises three pairs of mutations as defined in a), b) and c), above.

The obligate heterodimer meganuclease consists advantageously of a first monomer (A) having at least the mutations (i) E8R, E8K or E8H, E61R, E61K or E61H and L97F, L97W or L97Y; (ii) K7R, E8R, E61R, K96R and L97F, or (iii) K7R, E8R, F54W, E61R, K96R and L97F and a second monomer (B) having at least the mutations (iv) K7E or K7D, F54G or F54A and K96D or K96E; (v) K7E, F54G, L58M and K96E, or (vi) K7E, F54G, K57M and K96E. For example, the first monomer may have the mutations K7R, E8R or E8K, E61R, K96R and L97F or K7R, E8R or E8K, F54W, E61R, K96R and L97F and the second monomer, the mutations K7E, F54G, L58M and K96E or K7E, F54G, K57M and K96E. The obligate heterodimer may comprise at least one NLS and/or one tag as defined above; said NLS and/or tag may be in the first and/or the second monomer.

The subject-matter of the present invention is also a single-chain chimeric meganuclease (fusion protein) derived from an I-CreI variant as defined above. The single-chain meganuclease may comprise two I-CreI monomers, two I-CreI core domains (positions 6 to 94 of I-CreI) or a combination of both. Preferably, the two monomers/core domains or the combination of both, are connected by a peptidic linker. Said peptidic linker can be RM2 linker (SEQ ID NO: 78) or another suitable linker. More preferably the single-chain chimeric meganuclease is composed by one of the possible associations between variants from the group consisting of SEQ ID NO: 40 to 52, SEQ ID NO: 53 to 65, SEQ ID NO: 92 to 103 and SEQ ID NO: 105 to 116 connected by a linker. More preferably this single-chain chimeric meganuclease is one from the group consisting of SEQ ID NO: 66 to 76, SEQ ID NO: 104 and SEQ ID NO: 117 to 123.

It is understood that the scope of the present invention also encompasses the I-CreI variants per se, including heterodimers, obligate heterodimers, single chain meganucleases as non limiting examples, able to cleave one of the sequence targets in RHO gene.

The subject-matter of the present invention is also a polynucleotide fragment encoding a variant or a single-chain chimeric meganuclease as defined above; said polynucleotide may encode one monomer of a homodimeric or heterodimeric variant, or two domains/monomers of a single-chain chimeric meganuclease. It is understood that the subject-matter of the present invention is also a polynucleotide fragment encoding one of the variant species as defined above, obtained by any method well-known in the art.

The subject-matter of the present invention is also a recombinant vector for the expression of a variant or a single-chain meganuclease according to the invention. The recombinant vector comprises at least one polynucleotide fragment encoding a variant or a single-chain meganuclease, as defined above. In a preferred embodiment, said vector comprises two different polynucleotide fragments, each encoding one of the monomers of a heterodimeric variant.

A vector which can be used in the present invention includes, but is not limited to, a viral vector, a plasmid, a RNA vector or a linear or circular DNA or RNA molecule which may consists of a chromosomal, non chromosomal, semi-synthetic or synthetic nucleic acids. Preferred vectors are those capable of autonomous replication (episomal vector) and/or expression of nucleic acids to which they are linked (expression vectors). Large numbers of suitable vectors are known to those skilled in the art and commercially available.

Viral vectors include retrovirus, adenovirus, parvovirus (e.g. adeno-associated viruses), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D type viruses, HTLV-BLV group, lentivirus (particularly self inactivacting lentiviral vectors), spumavirus (Coffin, J. M., Retroviridae: The viruses and their replication, In Fundamental Virology, Third Edition, B. N. Fields, et al., Eds., Lippincott-Raven Publishers, Philadelphia, 1996).

Preferred vectors include adeno-associated viruses (AAV) based on existing studies on RHO gene transfer into retinal cells.

Vectors can comprise selectable markers, for example: neomycin phosphotransferase, histidinol dehydrogenase, dihydrofolate reductase, hygromycin phosphotransferase, herpes simplex virus thymidine kinase, adenosine deaminase, Glutamine Synthetase, and hypoxanthine-guanine phosphoribosyl transferase for eukaryotic cell culture; TRP1, URA3 and LEU2 for S. cerevisiae; tetracycline, rifampicin or ampicillin resistance in E. coli.

Preferably said vectors are expression vectors, wherein the sequence(s) encoding the variant/single-chain meganuclease of the invention is placed under control of appropriate transcriptional and translational control elements to permit production or synthesis of said variant. Therefore, said polynucleotide is comprised in an expression cassette. More particularly, the vector comprises a replication origin, a promoter operatively linked to said polynucleotide, a ribosome-binding site, an RNA-splicing site (when genomic DNA is used), a polyadenylation site and a transcription termination site. It also can comprise an enhancer. Selection of the promoter will depend upon the cell in which the polypeptide is expressed. Preferably, when said variant is a heterodimer, the two polynucleotides encoding each of the monomers are included in one vector which is able to drive the expression of both polynucleotides, simultaneously. Suitable promoters include tissue specific and/or inducible promoters. Examples of inducible promoters are: eukaryotic metallothionine promoter which is induced by increased levels of heavy metals, prokaryotic lacZ promoter which is induced in response to isopropyl-β-D-thiogalacto-pyranoside (IPTG) and eukaryotic heat shock promoter which is induced by increased temperature. Examples of tissue specific promoters are skeletal muscle creatine kinase, prostate-specific antigen (PSA), α-antitrypsin protease, human surfactant (SP) A and B proteins, β-casein and acidic whey protein genes.

According to another advantageous embodiment of said vector, it includes a targeting construct comprising sequences sharing homologies with the region surrounding the genomic DNA cleavage site as defined above.

For instance, said sequence sharing homologies with the regions surrounding the genomic DNA cleavage site of the variant is a fragment of the human RHO. Alternatively, the vector coding for an I-CreI variant/single-chain meganuclease and the vector comprising the targeting construct are different vectors.

More preferably, the targeting DNA construct comprises:

a) sequences sharing homologies with the region surrounding the genomic DNA cleavage site as defined above, and

b) a sequence to be introduced flanked by sequences as in a) or included in sequences as in a).

Preferably, homologous sequences of at least 50 bp, preferably more than 100 bp and more preferably more than 200 bp are used. Therefore, the targeting DNA construct is preferably from 200 bp to 6000 bp, more preferably from 1000 bp to 2000 bp. Indeed, shared DNA homologies are located in regions flanking upstream and downstream the site of the break and the DNA sequence to be introduced should be located between the two arms. The sequence to be introduced may be any sequence used to alter the chromosomal DNA in some specific way including a sequence used to repair a mutation in the RHO gene, restore a functional RHO gene in place of a mutated one, modify a specific sequence in the RHO gene, to attenuate or activate the RHO gene, to inactivate or delete the RHO gene or part thereof, to introduce a mutation into a site of interest or to introduce an exogenous gene or part thereof. Such chromosomal DNA alterations are used for genome engineering (animal models/recombinant cell lines) or genome therapy (gene correction or recovery of a functional gene). The targeting construct comprises advantageously a positive selection marker between the two homology arms and eventually a negative selection marker upstream of the first homology arm or downstream of the second homology arm. The marker(s) allow(s) the selection of cells having inserted the sequence of interest by homologous recombination at the target site.

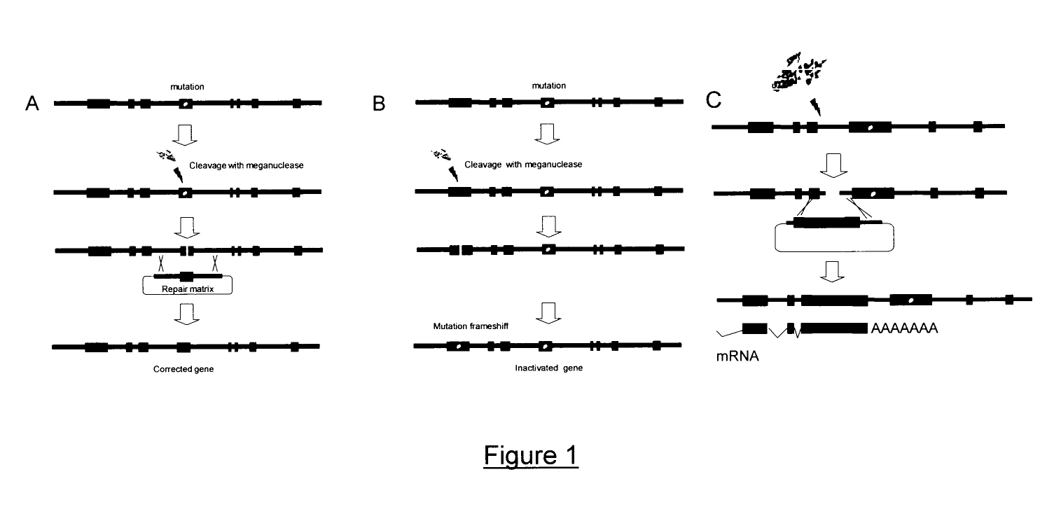

The sequence to be introduced is a sequence which repairs a mutation in the RHO gene (gene correction or recovery of a functional gene), for the purpose of genome therapy (FIGS. 1A and 1C). For correcting the RHO gene, cleavage of the gene occurs in the vicinity of the mutation, preferably, within 500 bp of the mutation (FIG. 1C). The targeting construct comprises a RHO gene fragment which has at least 200 bp of homologous sequence flanking the target site (minimal repair matrix) for repairing the cleavage, and includes a sequence encoding a portion of wild-type RHO gene corresponding to the region of the mutation for repairing the mutation (FIG. 1C). Consequently, the targeting construct for gene correction comprises or consists of the minimal repair matrix; it is preferably from 200 pb to 6000 pb, more preferably from 1000 pb to 2000 pb. Preferably, when the cleavage site of the variant overlaps with the mutation the repair matrix includes a modified cleavage site that is not cleaved by the variant which is used to induce said cleavage in the RHO gene and a sequence encoding wild-type RHO that does not change the open reading frame of the RHO gene.

Alternatively, for the generation of knock-in cells/animals, the targeting DNA construct may comprise flanking regions corresponding to RHO gene fragments which has at least 200 bp of homologous sequence flanking the target site of the I-CreI variant for repairing the cleavage, an exogenous gene of interest within an expression cassette and eventually a selection marker such as the neomycin resistance gene.

For the insertion of a sequence, DNA homologies are generally located in regions directly upstream and downstream to the site of the break (sequences immediately adjacent to the break; minimal repair matrix). However, when the insertion is associated with a deletion of ORF sequences flanking the cleavage site, shared DNA homologies are located in regions upstream and downstream the region of the deletion.

Alternatively, for restoring a functional gene (FIGS. 1A et 1C), cleavage of the gene occurs in the vicinity or upstream of a mutation. Preferably said mutation is the first known mutation in the sequence of the gene, so that all the downstream mutations of the gene can be corrected simultaneously. The targeting construct comprises the exons downstream of the cleavage site fused in frame (as in the cDNA) and with a polyadenylation site to stop transcription in 3′. The sequence to be introduced (exon knock-in construct) is flanked by introns or exons sequences surrounding the cleavage site, so as to allow the transcription of the engineered gene (exon knock-in gene) into a mRNA able to code for a functional protein (FIG. 1C). For example, the exon knock-in construct is flanked by sequences upstream and downstream of the cleavage site, from a minimal repair matrix as defined above.

The subject matter of the present invention is also a targeting DNA construct as defined above.

The subject-matter of the present invention is also a composition characterized in that it comprises at least one meganuclease as defined above (variant or single-chain chimeric meganuclease) and/or at least one expression vector encoding said meganuclease, as defined above.

In a preferred embodiment of said composition, it comprises a targeting DNA construct, as defined above.

Preferably, said targeting DNA construct is either included in a recombinant vector or it is included in an expression vector comprising the polynucleotide(s) encoding the meganuclease according to the invention.

The subject-matter of the present invention is further the use of a meganuclease as defined above, one or two polynucleotide(s), preferably included in expression vector(s), for repairing mutations of the RHO gene.

According to an advantageous embodiment of said use, it is for inducing a double-strand break in a site of interest of the RHO gene comprising a genomic DNA target sequence, thereby inducing a DNA recombination event, a DNA loss or cell death.

According to the invention, said double-strand break is for: repairing a specific sequence in the RHO gene, modifying a specific sequence in the RHO gene, restoring a functional RHO gene in place of a mutated one, attenuating or activating the RHO gene, introducing a mutation into a site of interest of the RHO gene, introducing an exogenous gene or a part thereof, inactivating or deleting the RHO gene or a part thereof, translocating a chromosomal arm, or leaving the DNA unrepaired and degraded.

The subject-matter of the present invention is also a method for making a RHO knock-out or knock-in recombinant cell, comprising at least the step of:

(a) introducing into a cell, a meganuclease as defined above (I-CreI variant or single-chain derivative), so as to induce a double stranded cleavage at a site of interest of the RHO gene comprising a DNA recognition and cleavage site for said meganuclease, simultaneously or consecutively,

(b) introducing into the cell of step (a), a targeting DNA, wherein said targeting DNA comprises (1) DNA sharing homologies to the region surrounding the cleavage site and (2) DNA which repairs the site of interest upon recombination between the targeting DNA and the chromosomal DNA, so as to generate a recombinant cell having repaired the site of interest by homologous recombination,

(c) isolating the recombinant cell of step (b), by any appropriate means.

The subject-matter of the present invention is also a method for making a RHO knock-out or knock-in animal, comprising at least the step of:

(a) introducing into a pluripotent precursor cell or an embryo of an animal, a meganuclease as defined above, so as to induce a double stranded cleavage at a site of interest of the RHO gene comprising a DNA recognition and cleavage site for said meganuclease, simultaneously or consecutively,

(b) introducing into the animal precursor cell or embryo of step (a) a targeting DNA, wherein said targeting DNA comprises (1) DNA sharing homologies to the region surrounding the cleavage site and (2) DNA which repairs the site of interest upon recombination between the targeting DNA and the chromosomal DNA, so as to generate a genetically modified animal precursor cell or embryo having repaired the site of interest by homologous recombination,

(c) developing the genetically modified animal precursor cell or embryo of step (b) into a chimeric animal, and

(d) deriving a transgenic animal from the chimeric animal of step (c).

Preferably, step (c) comprises the introduction of the genetically modified precursor cell generated in step (b) into blastocysts so as to generate chimeric animals.

The targeting DNA is introduced into the cell under conditions appropriate for introduction of the targeting DNA into the site of interest.

For making knock-out cells/animals, the DNA which repairs the site of interest comprises sequences that inactivate the RHO gene.

For making knock-in cells/animals, the DNA which repairs the site of interest comprises the sequence of an exogenous gene of interest, and eventually a selection marker, such as the neomycin resistance gene.

In a preferred embodiment, said targeting DNA construct is inserted in a vector.

The subject-matter of the present invention is also a method for making a RHO-deficient cell, comprising at least the step of:

(a) introducing into a cell, a meganuclease as defined above, so as to induce a double stranded cleavage at a site of interest of the RHO gene comprising a DNA recognition and cleavage site of said meganuclease, and thereby generate genetically modified RHO deficient cell having repaired the double-strands break, by non-homologous end joining, and

(b) isolating the genetically modified RHO deficient cell of step (a), by any appropriate mean.

The subject-matter of the present invention is also a method for making a RHO knock-out animal, comprising at least the step of:

(a) introducing into a pluripotent precursor cell or an embryo of an animal, a meganuclease, as defined above, so as to induce a double stranded cleavage at a site of interest of the RHO gene comprising a DNA recognition and cleavage site of said meganuclease, and thereby generate genetically modified precursor cell or embryo having repaired the double-strands break by non-homologous end joining,

(b) developing the genetically modified animal precursor cell or embryo of step (a) into a chimeric animal, and

(c) deriving a transgenic animal from a chimeric animal of step (b).

Preferably, step (b) comprises the introduction of the genetically modified precursor cell obtained in step (a), into blastocysts, so as to generate chimeric animals.

The cells which are modified may be any cells of interest as long as they contain the specific target site. For making knock-in/transgenic mice, the cells are pluripotent precursor cells such as embryo-derived stem (ES) cells, which are well-known in the art. For making recombinant human cell lines, the cells may advantageously be PerC6 (Fallaux et al., Hum. Gene Ther. 9, 1909-1917, 1998) or HEK293 (ATCC # CRL-1573) cells.

The animal is preferably a mammal, more preferably a laboratory rodent (mice, rat, guinea-pig), or a rabbit, a cow, pig, horse or goat.

Said meganuclease can be provided directly to the cell or through an expression vector comprising the polynucleotide sequence encoding said meganuclease and suitable for its expression in the used cell.

For making recombinant cell lines expressing an heterologous protein of interest, the targeting DNA comprises a sequence encoding the product of interest (protein or RNA), and eventually a marker gene, flanked by sequences upstream and downstream the cleavage site, as defined above, so as to generate genetically modified cells having integrated the exogenous sequence of interest in the RHO gene, by homologous recombination.

The sequence of interest may be any gene coding for a certain protein/peptide of interest, included but not limited to: reporter genes, receptors, signaling molecules, transcription factors, pharmaceutically active proteins and peptides, disease causing gene products and toxins. The sequence may also encode a RNA molecule of interest including for example an interfering RNA such as ShRNA, miRNA or siRNA, well-known in the art.

The expression of the exogenous sequence may be driven, either by the endogenous Rho gene promoter or by a heterologous promoter, preferably a ubiquitous or tissue specific promoter, either constitutive or inducible, as defined above. In addition, the expression of the sequence of interest may be conditional; the expression may be induced by a site-specific recombinase such as Cre or FLP (Akagi K, Sandig V, Vooijs M, Van der Valk M, Giovannini M, Strauss M, Berns A (May 1997). “Nucleic Acids Res. 25 (9): 1766-73.; Zhu X D, Sadowski P D (1995). J Biol Chem 270).

Thus, the sequence of interest is inserted in an appropriate cassette that may comprise an heterologous promoter operatively linked to said gene of interest and one or more functional sequences including but not limited to (selectable) marker genes, recombinase recognition sites, polyadenylation signals, splice acceptor sequences, introns, tag for protein detection and enhancers.

The subject matter of the present invention is also a kit for making RHO knock-out or knock-in cells/animals comprising at least a meganuclease and/or one expression vector, as defined above. Preferably, the kit further comprises a targeting DNA comprising a sequence that inactivates the RHO gene flanked by sequences sharing homologies with the region of the RHO gene surrounding the DNA cleavage site of said meganuclease. In addition, for making knock-in cells/animals, the kit includes also a vector comprising a sequence of interest to be introduced in the genome of said cells/animals and eventually a selectable marker gene, as defined above.

The subject-matter of the present invention is also the use of at least one meganuclease and/or one expression vector, as defined above, for the preparation of a medicament for preventing, improving or curing a pathological condition caused by a mutation in the RHO gene as defined above, in an individual in need thereof.

Preferably said pathological condition is a group of inherited retinal degenerative disorders characterized by progressive degeneration of the midperipheral retina, leading to night blindness, visual field constriction, and eventual loss of visual acuity, known as Retinitis Pigmentosa. More preferably, said pathological condition is the autosomal dominant inherited form of Retinitis Pigmentosa (adRP).

Since RHO mutations have also been associated with other milder retinal pathologies such as autosomal dominant Congenital stationary night blindness (AdCSNB, Zeitz et al), the development of meganucleases might prove useful in the context of other pathologies whenever Rho mutations are or will be reported (retinopathies, rod-cone dystrophies).

The use of the meganuclease may comprise at least the step of (a) inducing in somatic tissue(s) of the donor/individual a double stranded cleavage at a site of interest of the RHO gene comprising at least one recognition and cleavage site of said meganuclease by contacting said cleavage site with said meganuclease, and (b) introducing into said somatic tissue(s) a targeting DNA, wherein said targeting DNA comprises (1) DNA sharing homologies to the region surrounding the cleavage site and (2) DNA which repairs the RHO gene upon recombination between the targeting DNA and the chromosomal DNA, as defined above. The targeting DNA is introduced into the somatic tissues(s) under conditions appropriate for introduction of the targeting DNA into the site of interest.

According to the present invention, said double-stranded cleavage may be induced, ex vivo by introduction of said meganuclease into somatic cells from the diseased individual and then transplantation of the modified cells back into the diseased individual.

The subject-matter of the present invention is also a method for preventing, improving or curing a pathological condition caused by a mutation in the RHO gene, in an individual in need thereof, said method comprising at least the step of administering to said individual a composition as defined above, by any means. The meganuclease can be used either as a polypeptide or as a polynucleotide construct encoding said polypeptide. It is introduced into mouse cells, by any convenient means well-known to those in the art, which are appropriate for the particular cell type, alone or in association with either at least an appropriate vehicle or carrier and/or with the targeting DNA.

According to an advantageous embodiment of the uses according to the invention, the meganuclease (polypeptide) is associated with:
liposomes, polyethyleneimine (PEI); in such a case said association is administered and therefore introduced into somatic target cells.
membrane translocating peptides (Bonetta, The Scientist, 2002, 16, 38; Ford et al., Gene Ther., 2001, 8, 1-4; Wadia and Dowdy, Curr. Opin. Biotechnol., 2002, 13, 52-56); in such a case, the sequence of the variant/single-chain meganuclease is fused with the sequence of a membrane translocating peptide (fusion protein).

According to another advantageous embodiment of the uses according to the invention, the meganuclease (polynucleotide encoding said meganuclease) and/or the targeting DNA is inserted in a vector. Vectors comprising targeting DNA and/or nucleic acid encoding a meganuclease can be introduced into a cell by a variety of methods (e.g., injection, direct uptake, projectile bombardment, liposomes, electroporation). Meganucleases can be stably or transiently expressed into cells using expression vectors. Techniques of expression in eukaryotic cells are well known to those in the art. (See Current Protocols in Human Genetics: Chapter 12 “Vectors For Gene Therapy” & Chapter 13 “Delivery Systems for Gene Therapy”). Optionally, it may be preferable to incorporate a nuclear localization signal into the recombinant protein to be sure that it is expressed within the nucleus.

Once in a cell, the meganuclease and if present, the vector comprising targeting DNA and/or nucleic acid encoding a meganuclease are imported or translocated by the cell from the cytoplasm to the site of action in the nucleus.

Rhodopsin is a visual pigment which is highly expressed in vertebrate retinal rod cells (Zeitz et al) and is thus a retina associated gene. Meganuclease targeting the Rho gene, especially the meganucleases whose sites are located close to the Rho promoter region, could be used to insert genetic elements (transgenes, tags, reporter genes) under the control of Rho promoter allowing targeted expression in the retina. The generation of Knock out models [ips (induced pluripotent stem cells), cell lines or animal models] for Rho gene could be envisioned via NHEJ gene inactivation approach.

The CMV promoter has been successfully used to express transgene in cells of the retina (Takahashi et al). The pCLS 1853 backbone of the mammalian expression vector used for SCOH meganuclease testing in CHO SSA Assay bears the CMV promoter and should be suitable for meganuclease expression in target cells of the retina. Since AAV vectorization should provide long term expression of the meganuclease the use of inducible expression systems might be a strategic option. The possibility to use inducible expression systems has been demonstrated in the eye with tet-on inducible expression system (Gimenez et al).

Since meganucleases recognize a specific DNA sequence, any meganuclease developed in the context of human Rho gene therapy could be used in other contexts (other organisms, other loci, use in the context of a landing pad containing the site) unrelated with gene therapy of rhodopsin in human as long as the site is present.

For purposes of therapy, the meganucleases and a pharmaceutically acceptable excipient are administered in a therapeutically effective amount. Such a combination is said to be administered in a “therapeutically effective amount” if the amount administered is physiologically significant. An agent is physiologically significant if its presence results in a detectable change in the physiology of the recipient. In the present context, an agent is physiologically significant if its presence results in a decrease in the severity of one or more symptoms of the targeted disease and in a genome correction of the lesion or abnormality. Vectors comprising targeting DNA and/or nucleic acid encoding a meganuclease can be introduced into a cell by a variety of methods (e.g., injection, direct uptake, projectile bombardment, liposomes, electroporation). Meganucleases can be stably or transiently expressed into cells using expression vectors. Techniques of expression in eukaryotic cells are well known to those in the art. (See Current Protocols in Human Genetics: Chapter 12 “Vectors For Gene Therapy” & Chapter 13 “Delivery Systems for Gene Therapy”).

In one embodiment of the uses according to the present invention, the meganuclease is substantially non-immunogenic, i.e., engender little or no adverse immunological response. A variety of methods for ameliorating or eliminating deleterious immunological reactions of this sort can be used in accordance with the invention. In a preferred embodiment, the meganuclease is substantially free of N-formyl methionine. Another way to avoid unwanted immunological reactions is to conjugate meganucleases to polyethylene glycol (“PEG”) or polypropylene glycol (“PPG”) (preferably of 500 to 20,000 daltons average molecular weight (MW)). Conjugation with PEG or PPG, as described by Davis et al. (U.S. Pat. No. 4,179,337) for example, can provide non-immunogenic, physiologically active, water soluble endonuclease conjugates with anti-viral activity. Similar methods also using a polyethylene-polypropylene glycol copolymer are described in Saifer et al. (U.S. Pat. No. 5,006,333).

The invention also concerns a prokaryotic or eukaryotic host cell which is modified by a polynucleotide or a vector as defined above, preferably an expression vector.

The invention also concerns a non-human transgenic animal or a transgenic plant, characterized in that all or a part of their cells are modified by a polynucleotide or a vector as defined above.

As used herein, a cell refers to a prokaryotic cell, such as a bacterial cell, or an eukaryotic cell, such as an animal, plant or yeast cell.

The subject-matter of the present invention is also the use of at least one meganuclease variant, as defined above, as a scaffold for making other meganucleases. For example, further rounds of mutagenesis and selection/screening can be performed on said variants, for the purpose of making novel meganucleases.

The different uses of the meganuclease and the methods of using said meganuclease according to the present invention include the use of the I-CreI variant, the single-chain chimeric meganuclease derived from said variant, the polynucleotide(s), vector, cell, transgenic plant or non-human transgenic mammal encoding said variant or single-chain chimeric meganuclease, as defined above.

The subject matter of the present invention is also an I-CreI variant having mutations at positions 28 to 40 and/or 44 to 77 of I-CreI that is useful for engineering the variants able to cleave a DNA target from the RHO gene, according to the present invention. In particular, the invention encompasses the I-CreI variants as defined in step (c) to (f) of the method for engineering I-CreI variants, as defined above, including the variants at positions 28, 30, 32, 33, 38 and 40, or 44, 68, 70, 75 and 77. The invention encompasses also the I-CreI variants as defined in step (g), (h), (i), (j), (k) and (l) of the method for engineering I-CreI variants, as defined above including the variants of Tables I and III and Tables IV to XIII.

Single-chain chimeric meganucleases able to cleave a DNA target from the gene of interest are derived from the variants according to the invention by methods well-known in the art (Epinat et al., Nucleic Acids Res., 2003, 31, 2952-62; Chevalier et al., Mol. Cell., 2002, 10, 895-905; Steuer et al., Chembiochem., 2004, 5, 206-13; International PCT Applications WO 03/078619, WO 2004/031346 and WO 2009/095793). Any of such methods, may be applied for constructing single-chain chimeric meganucleases derived from the variants as defined in the present invention. In particular, the invention encompasses also the I-CreI variants defined in the tables II, IV, VIII and XIII.

The polynucleotide sequence(s) encoding the variant as defined in the present invention may be prepared by any method known by the man skilled in the art. For example, they are amplified from a cDNA template, by polymerase chain reaction with specific primers. Preferably the codons of said cDNA are chosen to favour the expression of said protein in the desired expression system.

The recombinant vector comprising said polynucleotides may be obtained and introduced in a host cell by the well-known recombinant DNA and genetic engineering techniques.

The I-CreI variant or single-chain derivative as defined in the present invention are produced by expressing the polypeptide(s) as defined above; preferably said polypeptide(s) are expressed or co-expressed (in the case of the variant only) in a host cell or a transgenic animal/plant modified by one expression vector or two expression vectors (in the case of the variant only), under conditions suitable for the expression or co-expression of the polypeptide(s), and the variant or single-chain derivative is recovered from the host cell culture or from the transgenic animal/plant.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Current Protocols in Molecular Biology (Frederick M. AUSUBEL, 2000, Wiley and son Inc, Library of Congress, USA); Molecular Cloning: A Laboratory Manual, Third Edition, (Sambrook et al, 2001, Cold Spring Harbor, N.Y.: Cold Spring Harbor Laboratory Press); Oligonucleotide Synthesis (M. J. Gait ed., 1984); Mullis et al. U.S. Pat. No. 4,683,195; Nucleic Acid Hybridization (B. D. Harries & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the series, Methods In ENZYMOLOGY (J. Abelson and M. Simon, eds.-in-chief, Academic Press, Inc., New York), specifically, Vols. 154 and 155 (Wu et al. eds.) and Vol. 185, “Gene Expression Technology” (D. Goeddel, ed.); Gene Transfer Vectors For Mammalian Cells (J. H. Miller and M. P. Calos eds., 1987, Cold Spring Harbor Laboratory); Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); and Manipulating the Mouse Embryo, (Cold Spring Harbor Laboratory Press, Cold Spring Harbor, N.Y., 1986).

### DEFINITIONS

Amino acid residues in a polypeptide sequence are designated herein according to the one-letter code, in which, for example, Q means Gln or Glutamine residue, R means Arg or Arginine residue and D means Asp or Aspartic acid residue.
Amino acid substitution means the replacement of one amino acid residue with another, for instance the replacement of an Arginine residue with a Glutamine residue in a peptide sequence is an amino acid substitution.
Altered/enhanced/increased cleavage activity, refers to an increase in the detected level of meganuclease cleavage activity, see below, against a target DNA sequence by a second meganuclease in comparison to the activity of a first meganuclease against the target DNA sequence. Normally the second meganuclease is a variant of the first and comprise one or more substituted amino acid residues in comparison to the first meganuclease.
Nucleotides are designated as follows: one-letter code is used for designating the base of a nucleoside: a is adenine, t is thymine, c is cytosine, and g is guanine. For the degenerated nucleotides, r represents g or a (purine nucleotides), k represents g or t, s represents g or c, w represents a or t, m represents a or c, y represents t or c (pyrimidine nucleotides), d represents g, a or t, v represents g, a or c, b represents g, t or c, h represents a, t or c, and n represents g, a, t or c.
by “meganuclease”, is intended an endonuclease having a double-stranded DNA target sequence of 12 to 45 bp. Said meganuclease is either a dimeric enzyme, wherein each domain is on a monomer or a monomeric enzyme comprising the two domains on a single polypeptide.
by “meganuclease domain” is intended the region which interacts with one half of the DNA target of a meganuclease and is able to associate with the other domain of the same meganuclease which interacts with the other half of the DNA target to form a functional meganuclease able to cleave said DNA target.
by “meganuclease variant” or “variant” it is intended a meganuclease obtained by replacement of at least one residue in the amino acid sequence of the parent meganuclease with a different amino acid.
by “peptide linker” it is intended to mean a peptide sequence of at least 10 and preferably at least 17 amino acids which links the C-terminal amino acid residue of the first monomer to the N-terminal residue of the second monomer and which allows the two variant monomers to adopt the correct conformation for activity and which does not alter the specificity of either of the monomers for their targets.
by “subdomain” it is intended the region of a LAGLIDADG homing endonuclease core domain which interacts with a distinct part of a homing endonuclease DNA target half-site.
by “targeting DNA construct/minimal repair matrix/repair matrix” it is intended to mean a DNA construct comprising a first and second portions which are homologous to regions 5′ and 3′ of the DNA target in situ. The DNA construct also comprises a third portion positioned between the first and second portion which comprise some homology with the corresponding DNA sequence in situ or alternatively comprise no homology with the regions 5′ and 3′ of the DNA target in situ. Following cleavage of the DNA target, a homologous recombination event is stimulated between the genome containing the RHO gene and the repair matrix, wherein the genomic sequence containing the DNA target is replaced by the third portion of the repair matrix and a variable part of the first and second portions of the repair matrix.
by “functional variant” is intended a variant which is able to cleave a DNA target sequence, preferably said target is a new target which is not cleaved by the parent meganuclease. For example, such variants have amino acid variation at positions contacting the DNA target sequence or interacting directly or indirectly with said DNA target.
by “selection or selecting” it is intended to mean the isolation of one or more meganuclease variants based upon an observed specified phenotype, for instance altered cleavage activity. This selection can be of the variant in a peptide form upon which the observation is made or alternatively the selection can be of a nucleotide coding for selected meganuclease variant.
by “screening” it is intended to mean the sequential or simultaneous selection of one or more meganuclease variant (s) which exhibits a specified phenotype such as altered cleavage activity.
by “derived from” it is intended to mean a meganuclease variant which is created from a parent meganuclease and hence the peptide sequence of the meganuclease variant is related to (primary sequence level) but derived from (mutations) the sequence peptide sequence of the parent meganuclease.
by “I-CreI” is intended the wild-type I-CreI having the sequence of pdb accession code 1g9y, corresponding to the sequence SEQ ID NO: 1 in the sequence listing.
by “I-CreI variant with novel specificity” is intended a variant having a pattern of cleaved targets different from that of the parent meganuclease. The terms “novel specificity”, “modified specificity”, “novel cleavage specificity”, “novel substrate specificity” which are equivalent and used indifferently, refer to the specificity of the variant towards the nucleotides of the DNA target sequence. In the present patent application all the I-CreI variants described comprise an additional Alanine after the first Methionine of the wild type I-CreI sequence (SEQ ID NO: 1). These variants also comprise two additional Alanine residues and an Aspartic Acid residue after the final Proline of the wild type I-CreI sequence. These additional residues do not affect the properties of the enzyme and to avoid confusion these additional residues do not affect the numeration of the residues in I-CreI or a variant referred in the present patent application, as these references exclusively refer to residues of the wild type I-CreI enzyme (SEQ ID NO: 1) as present in the variant, so for instance residue 2 of I-CreI is in fact residue 3 of a variant which comprises an additional Alanine after the first Methionine.
by “I-CreI site” is intended a 22 to 24 bp double-stranded DNA sequence which is cleaved by I-CreI. I-CreI sites include the wild-type non-palindromic I-CreI homing site and the derived palindromic sequences such as the sequence 5′-t₋₁₂c₋₁₁a₋₁₀a₋₉a₋₈a₋₇c₋₆g₋₅t₋₄c₋₃g₋₂t₋₁a₊₁c₊₂g₊₃a₊₄c₊₅g₊₆t₊₇t₊₈t₊₉t₊₁₀g₊₁₁a₊₁₂ (SEQ ID NO: 2), also called C1221 (FIGS. 3, 6 and 9).
by “domain” or “core domain” is intended the “LAGLIDADG homing endonuclease core domain” which is the characteristic α₁β₁β₂α₂β₃β₄α₃ fold of the homing endonucleases of the LAGLIDADG family, corresponding to a sequence of about one hundred amino acid residues. Said domain comprises four beta-strands (β₁β₂β₃β₄) folded in an anti-parallel beta-sheet which interacts with one half of the DNA target. This domain is able to associate with another LAGLIDADG homing endonuclease core domain which interacts with the other half of the DNA target to form a functional endonuclease able to cleave said DNA target. For example, in the case of the dimeric homing endonuclease I-CreI (163 amino acids), the LAGLIDADG homing endonuclease core domain corresponds to the residues 6 to 94.
by “subdomain” is intended the region of a LAGLIDADG homing endonuclease core domain which interacts with a distinct part of a homing endonuclease DNA target half-site.
by “chimeric DNA target” or “hybrid DNA target” it is intended the fusion of a different half of two parent meganuclease target sequences. In addition at least one half of said target may comprise the combination of nucleotides which are bound by at least two separate subdomains (combined DNA target).

by “beta-hairpin” is intended two consecutive beta-strands of the antiparallel beta-sheet of a LAGLIDADG homing endonuclease core domain (β₁β₂ or, β₃β₄) which are connected by a loop or a turn,
by “single-chain meganuclease”, “single-chain chimeric meganuclease”, “single-chain meganuclease derivative”, “single-chain chimeric meganuclease derivative” or “single-chain derivative” is intended a meganuclease comprising two LAGLIDADG homing endonuclease domains or core domains linked by a peptidic spacer. The single-chain meganuclease is able to cleave a chimeric DNA target sequence comprising one different half of each parent meganuclease target sequence.
by “DNA target”, “DNA target sequence”, “target sequence”, “target-site”, “target”, “site”, “site of interest”, “recognition site”, “recognition sequence”, “homing recognition site”, “homing site”, “cleavage site” is intended a 20 to 24 bp double-stranded palindromic, partially palindromic (pseudo-palindromic) or non-palindromic polynucleotide sequence that is recognized and cleaved by a LAGLIDADG homing endonuclease such as I-CreI, or a variant, or a single-chain chimeric meganuclease derived from I-CreI. These terms refer to a distinct DNA location, preferably a genomic location, at which a double stranded break (cleavage) is to be induced by the meganuclease. The DNA target is defined by the 5′ to 3′ sequence of one strand of the double-stranded polynucleotide, as indicate above for C1221. Cleavage of the DNA target occurs at the nucleotides at positions +2 and −2, respectively for the sense and the antisense strand. Unless otherwise indicated, the position at which cleavage of the DNA target by an I-Cre I meganuclease variant occurs, corresponds to the cleavage site on the sense strand of the DNA target.
by “DNA target half-site”, “half cleavage site” or half-site” is intended the portion of the DNA target which is bound by each LAGLIDADG homing endonuclease core domain.
by “chimeric DNA target” or “hybrid DNA target” is intended the fusion of different halves of two parent meganuclease target sequences. In addition at least one half of said target may comprise the combination of nucleotides which are bound by at least two separate subdomains (combined DNA target).
by “RHO gene” is intended a Rhodopsin gene, preferably the RHO gene of a vertebrate, more preferably the RHO gene of a mammal such as human. RHO gene sequences are available in sequence databases, such as the NCBI/GenBank database. The human Rhodopsin gene has been described in databanks as Gene RHO human NCBI NC000003 (NC000003.11 for the 10-JUN-2009 update). This coding sequence (CDS) can be obtained by joining (96..456), (2238..2406), (3613..3778), (3895..4134), (4970..5080), corresponding to exon1, exon2, exon3, exon4 and exon5 respectively. Additionally, regions upstream of the Rho gene (promoter) can be found in the contig. As described in Table IX.
by “DNA target sequence from the RHO gene”, “genomic DNA target sequence”, “genomic DNA cleavage site”, “genomic DNA target” or “genomic target” is intended a 22 to 24 bp sequence of a RHO gene as defined above, which is recognized and cleaved by a meganuclease variant or a single-chain chimeric meganuclease derivative.
by “parent meganuclease” it is intended to mean a wild type meganuclease or a variant of such a wild type meganuclease with identical properties or alternatively a meganuclease with some altered characteristic in comparison to a wild type version of the same meganuclease. In the present invention the parent meganuclease can refer to the initial meganuclease from which the first series of variants are derived in step (a) or the meganuclease from which the second series of variants are derived in step (b), or the meganuclease from which the third series of variants are derived in step (k).
by “vector” is intended a nucleic acid molecule capable of transporting another nucleic acid to which it has been linked.
by “homologous” is intended a sequence with enough identity to another one to lead to homologous recombination between sequences, more particularly having at least 95% identity, preferably 97% identity and more preferably 99%.
“identity” refers to sequence identity between two nucleic acid molecules or polypeptides. Identity can be determined by comparing a position in each sequence which may be aligned for purposes of comparison. When a position in the compared sequence is occupied by the same base, then the molecules are identical at that position. A degree of similarity or identity between nucleic acid or amino acid sequences is a function of the number of identical or matching nucleotides at positions shared by the nucleic acid sequences. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default setting.
by “mutation” is intended the substitution, deletion, insertion of one or more nucleotides/amino acids in a polynucleotide (cDNA, gene) or a polypeptide sequence. Said mutation can affect the coding sequence of a gene or its regulatory sequence. It may also affect the structure of the genomic sequence or the structure/stability of the encoded mRNA.

The above written description of the invention provides a manner and process of making and using it such that any person skilled in this art is enabled to make and use the same, this enablement being provided in particular for the subject matter of the appended claims, which make up a part of the original description.

As used above, the phrases “selected from the group consisting of,” “chosen from,” and the like include mixtures of the specified materials.

Where a numerical limit or range is stated herein, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

Having generally described this invention, a further understanding can be obtained by reference to certain specific examples, which are provided herein for purposes of illustration only, and are not intended to be limiting unless otherwise specified.

### EXAMPLES

### Example 1

### Engineering Meganucleases Targeting the Rho34 Locus

Rho34 is a locus comprising a 24 bp non-palindromic target (ACTTCCTCACGCTCTACGTCACCG also referred to as Rho34.1 target=SEQ ID NO: 8) that is present in the first exon of RHO gene (reference sequence NC000003.11 as described in 10062009 database update; start by 259-282, downstream of the ATG).

It can thus be used for several strategies:
inactivation of the gene (dominant negative pathologic allele) by NHEJ induced mutagenesis in the absence of repair matrix.
gene correction or gene modification (cell line engineering at Rho34 locus with reporter genes for example) in the presence of a repair matrix.
introduction of a functional cds to follow a exon KI strategy; Rho34 localization in the first exon of RHO gene makes it especially well suited to apply this strategy.

I-CreI heterodimers able to cleave target sequence Rho 34.1 (SEQ ID NO: 8) were identified using methods derived from those described in Chames et al. (Nucleic Acids Res., 2005, 33, e178), Arnould et al. (J. Mol. Biol., 2006, 355, 443-458), Smith et al. (Nucleic Acids Res., 2006, 34, e149), Arnould et al. (Arnould et al. J Mol. Biol. 2007 371:49-65). Active heterodimers on Rho34.1 target (=SEQ ID NO: 8) were identified in Yeast. These results were then used to design single-chain meganucleases directed against the target sequence SEQ ID NO: 8. These single-chain meganucleases were cloned into mammalian expression vectors and tested for Rho34.1 cleavage in CHO cells. Strong cleavage activity of the Rho34.1 target could be observed for these single chain molecules in mammalian cells.

### Example 1.1

### Identification of Meganucleases Cleaving Rho34

I-CreI variants potentially cleaving the Rho34.1 target sequence in heterodimeric form were constructed by genetic engineering. Pairs of such variants were then co-expressed in yeast. Upon co-expression, one obtains three molecular species, namely two homodimers and one heterodimer. It was then determined whether the heterodimers were capable of cutting Rho34.1 target sequence SEQ ID NO: 8.

a) Construction of Variants of the I-CreI Meganuclease Cleaving Palindromic Sequences Derived from the Rho34.1 Target Sequence

The Rho34 sequence is partially a combination of the 10TTC_P (SEQ ID NO: 4), 5CAC_P (SEQ ID NO: 6), 10GTG_P (SEQ ID NO: 5) and 5GTA_P (SEQ ID NO: 7) target sequences which are shown on FIG. 3. These sequences are cleaved by mega-nucleases obtained as described in International PCT applications WO 2006/097784 and WO 2006/097853, Arnould et al. (J. Mol. Biol., 2006, 355, 443-458) and Smith et al. (Nucleic Acids Res., 2006). Thus, Rho34 should be cleaved by combinatorial variants resulting from these previously identified meganucleases.

A series of targets were derived from Rho34 (FIG. 3). The palindromic targets, Rho34.5 (ACTTCCTCACGCTCGTGAGGAAGT=SEQ ID NO: 11) and Rho34.6 (CGGTGACGTAGCTCTACGTCACCG=SEQ ID NO: 13), should be cleaved by homodimeric proteins. Therefore, homodimeric I-CreI variants cleaving either the Rho34.5 palindromic target sequence of SEQ ID NO: 11 or the Rho34.6 palindromic target sequence of SEQ ID NO: 13 were constructed using methods derived from those described in Chames et al. (Nucleic Acids Res., 2005, 33, e 178), Arnould et al. (J. Mol. Biol., 2006, 355, 443-458), Smith et al. (Nucleic Acids Res., 2006, 34, e 149) and Arnould et al. (Arnould et al. J Mol. Biol. 2007 371:49-65).

b) Construction of Target Vector

An oligonucleotide of SEQ ID NO: 77, corresponding to the Rho34.1 target sequence flanked by gateway cloning sequences, was ordered from PROLIGO. This oligo has the following sequence:

TGGCATACAAGTTTACTTCCTCACGCTCTACGTCACCGCAATCGTC
TGTCA).

Double-stranded target DNA, generated by PCR amplification of the single stranded oligonucleotide, was cloned into the pCLS 1055 yeast reporter vector using the Gateway protocol (INVITROGEN).

Yeast reporter vector was transformed into the FYBL2-7B Saccharomyces cerevisiae strain having the following genotype: MAT a, ura3Δ851, trp1Δ63, leu2Δ1, lys2Δ202. The resulting strain corresponds to a reporter strain (MILLEGEN).

c) Co-Expression of Variants

The open reading frames coding for the variants cleaving the Rho34.5 or the Rho34.6 sequences were cloned into the pCLS542 and pCLS1107 expression vectors, respectively. Yeast DNA from these variants was extracted using standard protocols and was used to transform E. coli. The resulting plasmids were then used to co-transform yeast. Transformants were selected on synthetic medium lacking leucine and containing G418.

d) Mating of Meganucleases Coexpressing Clones and Screening in Yeast

Mating was performed using a colony gridder (QpixII, Genetix). Variants were gridded on nylon filters covering YPD plates, using a low gridding density (4-6 spots/cm²). A second gridding process was performed on the same filters to spot a second layer consisting of different reporter-harboring yeast strains for each target. Membranes were placed on solid agar YPD rich medium, and incubated at 30° C. for one night, to allow mating. Next, filters were transferred to synthetic medium, lacking leucine and tryptophan, adding G418, with galactose (2%) as a carbon source, and incubated for five days at 37° C., to select for diploids carrying the expression and target vectors. After 5 days, filters were placed on solid agarose medium with 0.02% X-Gal in 0.5 M sodium phosphate buffer, pH 7.0, 0.1% SDS, 6% dimethyl formamide (DMF), 7 mM β-mercaptoethanol, 1% agarose, and incubated at 37° C., to monitor β-galactosidase activity. Results were analyzed by scanning and quantification was performed using an appropriate software.

e) Results

Co-expression of different variants resulted in cleavage of the Rho34.1 target in all of the 40 tested combinations summarized in Table I herebelow. In this table, “+” indicates a functional combination on the Rho34 target sequence, i.e., the heterodimer is able to cleave the Rho34 target sequence. SEQ ID NO: 40 to 47 correspond to variants cleaving Rho34.5 target (SEQ ID NO: 11). SEQ ID NO: 48 to 52 correspond to variants cleaving Rho34.6 target (SEQ ID NO: 13).

TABLE I
I-CreI variants able to cleave Rho34.5 and Rho34.6 targets

| | | Amino acids positions and residues | | | | | | | |
|---|---|---|---|---|---|---|---|---|---|
| | | of the I-CreI variants cleaving the Rho34.5 target (SEQ ID NO: 11) | | | | | | | |
| | | 32T33C38 | | 31R32T33 | 1V32T33C | 32T33C38 | 32T33C38 | 23V32T33 | 32T33C38 |
| | | H44V68Y7 | 32T33C38 | C38H44V6 | 38H44V68 | H44V54S6 | H41S44V6 | C38H44V6 | H44V68Y7 |
| | | 0S75R77V | H44V68Y7 | 8Y70S75R | Y70S75R7 | 8Y70S75R | 8Y70S75R | 8Y70S72P | 0S75R77V |
| | | 100R | 0S75R77V | 77V | 7V | 77V | 77V | 75R77V | 153G |
| | | (SEQ ID | (SEQ ID | (SEQ ID | (SEQ ID | (SEQ ID | (SEQ ID | (SEQ ID | (SEQ ID |
| | | NO: 40) | NO: 41) | NO: 42) | NO: 43) | NO: 44) | NO: 45) | NO: 46) | NO: 47) |
| Amino acids | 6D32H33 | + | + | + | + | + | + | + | + |
| positions and | H38A44S4 | | | | | | | | |
| residues of the | 6G70S73 | | | | | | | | |
| ICreI variants | M75E77Y | | | | | | | | |
| cleaving the | 117G132V | | | | | | | | |
| Rho34.6 target | (SEQ ID | | | | | | | | |
| (SEQ ID NO: 13) | NO: 48) | | | | | | | | |
| | 32H33H38 | + | + | + | + | + | + | + | + |
| | A44S46G6 | | | | | | | | |
| | 6H70S73 | | | | | | | | |
| | M75E77Y | | | | | | | | |
| | (SEQ ID | | | | | | | | |
| | NO: 49) | | | | | | | | |
| | 32H33H38 | + | + | + | + | + | + | + | + |
| | A44S46G5 | | | | | | | | |
| | 9A70S73 | | | | | | | | |
| | M75E77C | | | | | | | | |
| | 80G | | | | | | | | |
| | (SEQ ID | | | | | | | | |
| | NO: 50) | | | | | | | | |
| | 32H33H38 | + | + | + | + | + | + | + | + |
| | A44S46G6 | | | | | | | | |
| | 6H70S73 | | | | | | | | |
| | M75E77Y | | | | | | | | |
| | 105A | | | | | | | | |
| | (SEQ ID | | | | | | | | |
| | NO: 51) | | | | | | | | |
| | 32H33H38 | + | + | + | + | + | + | + | + |
| | A44S46G6 | | | | | | | | |
| | 6H69N70S | | | | | | | | |
| | 73M75E77 | | | | | | | | |
| | Y110G | | | | | | | | |
| | (SEQ ID | | | | | | | | |
| | NO: 52) | | | | | | | | |

In conclusion, several heterodimeric I-CreI variants able to cleave Rho34 target sequence in yeast were identified.

### Example 1.2

### Validation of Rho34 Target Cleavage in an Extrachromosomal Model in CHO Cells by Covalent Assembly of Heterodimers as Single Chain and Improvement of Meganucleases Cleaving Rho34

I-CreI variants able to efficiently cleave the Rho34 target in yeast when forming heterodimers are described hereabove in example 1.1. In order to further assess the cleavage activity for the Rho34 target in CHO cells, synthetic single chain molecules based on several pairs of mutants identified in Yeast have been assayed using an extrachromosomal assay in CHO cells. The screen in CHO cells is a single-strand annealing (SSA) based assay where cleavage of the target by the meganucleases induces homologous recombination and expression of a LagoZ reporter gene (a derivative of the bacterial lacZ gene).

The M1×MA Rho34 heterodimer gives high cleavage activity in yeast. Rho34.5-MA is a Rho34.5 cutter that bears the following mutations in comparison with the I-CreI wild type sequence: 32T 33C 38H 44V 54S 68Y 70S 75R 77V. Rho34.6-M1 is a Rho34.6 cutter that bears the following mutations in comparison with the I-CreI wild type sequence: 32H 33H 38A 44S 46G 59A 70S 73M 75E 77C 80G.

Single chain constructs were engineered using the linker RM2 [AAGGSDKYNQALSKYNQALSKYNQALSGGGGS (SEQ ID NO: 78)], thus resulting in the production of the single chain molecule: MA-linkerRM2-M1. During this design step, the G19S mutation was introduced into the C-terminal M1 variant. In addition, mutations K7E and K96E were introduced into the MA variant and mutations E8K and E61R into the M1 variant to create the single chain molecule: MA (K7E K96E)-linkerRM2-M1 (E8K E61R G195) that is further called SCOH-ro34-b11 scaffold. Some additional amino-acid substitutions have been found in previous studies to enhance the activity of I-CreI derivatives: I132V (replacement of Isoleucine 132 with Valine), E80K and V105A are some of these mutations of potential interest. The I132V mutation was introduced into either one, both or none of the coding sequence of N-terminal and C-terminal protein fragments. In some cases, E80K and V105A mutations were also introduced as described in table II below.

The same strategy was applied to a second scaffold, termed SCOH-Ro34-b56 scaffold, based on the other variants cleaving Rho34.5 (32T 33C 38H 41S 44V 68Y 70S 75R 77V) and Rho34.6 (32H 33H 38A 44S 46G 66H 70S 73M 75E 77Y 105A) as homodimers, respectively.

The same strategy was applied to a third scaffold, termed SCOH-Ro34-b12 scaffold, based on another set of variants cleaving Rho34.5 (32T 33C 38H 44V 54S 68Y 70S 75R 77V) and Rho34.6 (32H 33H 38A 44S 46G 66H 70S 73M 75E 77Y 105A) as homodimers, respectively.

The resulting proteins are shown in Table II below. All the single chain molecules were assayed in CHO for cleavage of the Rho34 target.

a) Cloning of Rho34 Target in a Vector for CHO Screen

An oligonucleotide corresponding to the Rho34 target sequence flanked by gateway cloning sequences, was ordered from PROLIGO (TGGCATACAAGTTTACTTCCTCACGCTCTACGTCACCGCAATCGTCTGTCA=SEQ ID NO: 77). Double-stranded target DNA, generated by PCR amplification of the single stranded oligonucleotide, was cloned using the Gateway protocol (INVITROGEN) into the pCLS 1058 CHO reporter vector. Cloned target was verified by sequencing (MILLEGEN).

b) Cloning of the Single Chain Molecule

A series of synthetic gene assembly was ordered to MWG-EUROFINS. Synthetic genes coding for the different single chain variants targeting Rho34 (“SCOH-ro34”) were cloned into pCLS 1853 using AscI and XhoI restriction sites.

c) Extrachromosomal Assay in Mammalian Cells

CHO K1 cells were transfected with Polyfect® transfection reagent according to the supplier's protocol (Qiagen). 72 hours after transfection, culture medium was removed and 150 μl of lysis/revelation buffer for β-galactosidase liquid assay was added. After incubation at 37° C., OD was measured at 420 nm. The entire process was performed on an automated Velocity 11 BioCel platform. Per assay, 150 ng of target vector was cotransfected with an increasing quantity of variant DNA from 3.12 to 25 ng (25 ng of single chain DNA corresponding to 12.5 ng+12.5 ng of heterodimer DNA). Finally, the transfected DNA variant DNA quantity was 3.12 ng, 6.25 ng, 12.5 ng and 25 ng. The total amount of transfected DNA was completed to 175 ng (target DNA, variant DNA, carrier DNA) using an empty vector (pCLS0002).

d) Results

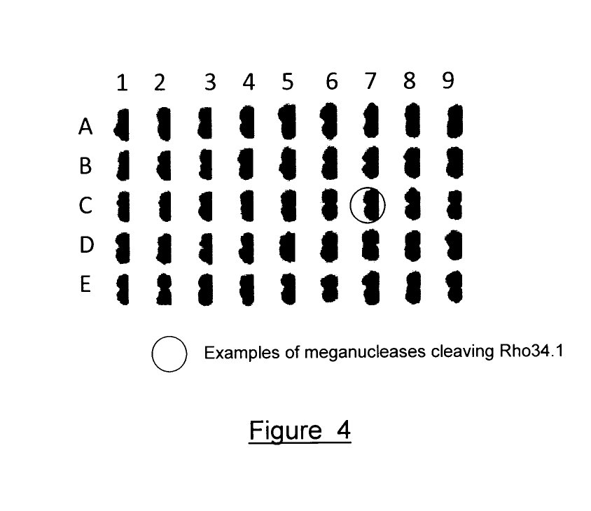

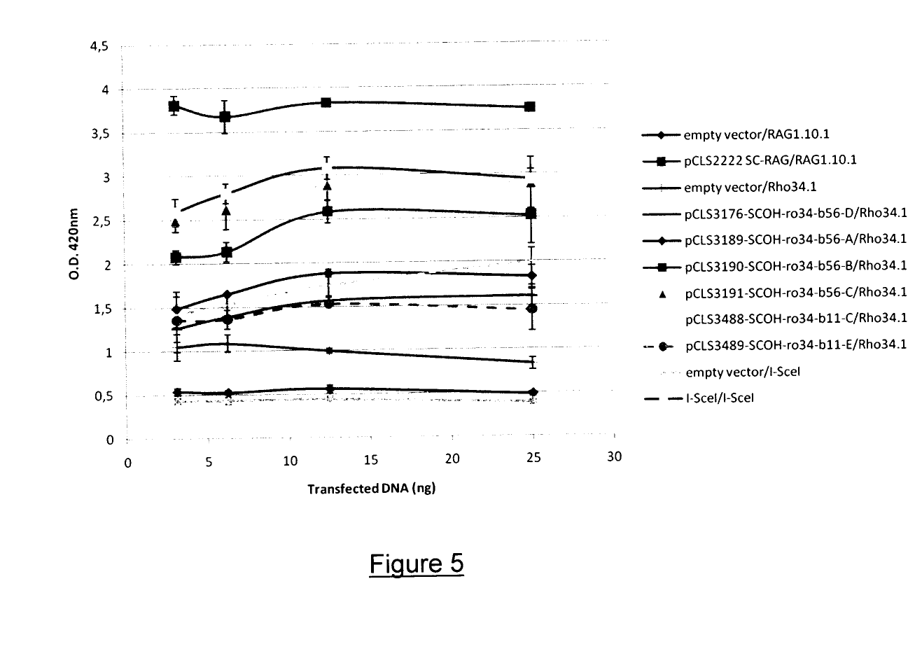

The activity of the single chain molecules against the Rho34 target was monitored using the previously described CHO assay along with our internal control SCOH-RAG and I-Sce I meganucleases. All comparisons were done at 3.12 ng, 6.25 ng, 12.5 ng, and 25 ng transfected variant DNA (FIGS. 4 and 5). Examples of single chain molecules displaying Rho34 target cleavage activity in CHO assay are listed in Table II below.

Variants shared specific behavior upon assayed dose depending on the mutation profile they bear (FIG. 5). For example, pCLS3191 SCOH-Ro34-b56-C displays higher activity at all tested doses than pCLS3488 SCOH-ro34-b11-C variant. pCLS3191 displays comparable level of activity as I-SceI a molecule known as a reference in genome engineering.

All of the “SCOH-ro34” variants active in CHO assay can be considered for genome engineering at Rho34 locus including insertion of transgenes, gene modification, gene correction and mutagenesis.

TABLE II
Single chain series designed for strong cleavage of Rho34 target in CHO cells

| | Mutations | Mutations | | |
|---|---|---|---|---|
| | on N- | on C- | | |
| | terminal | terminal | | SEQ |
| Name | monomer | monomer | Protein sequence | ID NO: |
| SCOH-ro34-b56-d | 7E32T33C38H | 8K19S32H33H | MANTKYNEEFLLYLAGFVDGDGSIIAQIKPNQTCKFKHHLSSTFVVTQ | 66 |
| (pCLS3176) | 41S44V68Y70 | 38A44S46G61 | KTQRRWFLDKLVDEIGVGYVYDSGSVSRYVLSEIKPLHNFLTQLQPFL | |
| | S75R77V96E | R66H70S73M | ELKQKQANLVLKIIEQLPSAKESPDKFLEVCTWVDQIAALNDSKTRKT | |
| | | 75E77Y105A1 | TSETVRAVLDSLSEKKKSSPAAGGSDKYNQALSKYNQALSKYNQALSG | |
| | | 32V | GGGSNKKFLLYLAGFVDSDGSIIAQIKPNQHHKFKHALSLTFSVGQKT | |
| | | | QRRWFLDKLVDRIGVGHVRDSGSMSEYYLSEIKPLHNFLTQLQPFLKL | |
| | | | KQKQANLALKIEEQLPSAKESPDKFLEVCTWVDQVAALDNSKTRKTTS | |
| | | | ETVRAVLDSLSEKKKSSP | |
| SCOH-ro34-b56-A | 7E32T33C38H | 8K19S32H33H | MANTKYNEEFLLYLAGFVDGDGSIIAQIKPNQTCKFKHHLSSTFVVTQ | 67 |
| (pCLS3189) | 41S44V68Y70 | 38A44S46G61 | KTQRRWFLDKLVDIEGVGYVYDSGSVSRYVLSEIKPLHNFLTQLQPFL | |
| | S75R77V96E | R66H70S73M | ELKQKQANLVLKIIEQLPSAKESPDKFLEVCTWVDQIAALNDSKTRKT | |
| | | 75E77Y105A | TSETVRAVLDSLSEKKKSSPAAGGSDKYNQALSKYNQALSKYNQALSG | |
| | | | GGGSNKKFLLYLAGFVDSDGSIIAQIKPNQHHKFKHALSLTFSVGQKT | |
| | | | QRRWFLDKLVDRIGVGHVRDSGSMSEYYLSEIKPLHNFLTQLQPFLKL | |
| | | | KQKQANLALKIEEQLPSAKESPDKFLEVCTWVDQIAALNDSKTRKTTS | |
| | | | ETVRAVLDSLSEKKKSSP | |
| SCOH-ro34-b56-B | 7E32T33C38H | 8K19S32H33H | MANTKYNEEFLLYLAGFVDGDGSIIAQIKPNQTCKFKHHLSSTFVVTQ | 68 |
| (pCLS3190) | 41S44V68Y70 | 38A44S46G61 | KTQRRWFLDKLVDEIGVGYVYDSGSVSRYVLSEIKPLHNFLTQLQPFL | |
| | S75R77V96E1 | R66H70S73M | ELKQKQANLVLKIIEQLPSAKESPDKFLEVCTWVDQVAALNDSKTRKT | |
| | 32V | 75E77Y105A | TSETVRAVLDSLSEKKKSSPAAGGSDKYNQALSKYNQALSKYNQALSG | |
| | | | GGGSNKKFLLYLAGFVVDSDGSIIAQIKPNQHHKFKHALSLTFSVGQK | |
| | | | TQRRWFLDKLVDRIGVGHVRDSGSMSEYYLSEIKPLHNFLTQLQPFLK | |
| | | | LKQKQANLALKIIEQLPSAKESPDKFLEVCTWVDQIAALNDSKTRKTT | |
| | | | SETVRAVLDSLSEKKKSSP | |
| SCOH-ro34-b56-C | 7E32T33C38H | 8K19S32H33H | MANTKYNEEFLLYLAGFVDGDGSIIAQIKPNQTCKFKHHLSSTFVVTQ | 69 |
| (pCLS3191) | 41S44V68Y70 | 38A44S46G61 | KTQRRWFLDKLVDEIGVGYVYDSGSVSRYVLSEIKPLHNFLTQLQPFL | |
| | S75R77V96E1 | R66H70S73M | ELKQKQANLVLKIIEQLPSAKESPDKFLEVCTWVDQVAALNDDKTRKT | |
| | 32V | 75E77Y105A1 | TSETVRAVLDSLSEKKKSSPAAGGSDKYNQALSKYNQALSKYNQALSG | |
| | | 32V | GGGSNKKFLLYLAGFVDSDGSIIAQIKPNQHHKFKHALSLTFSVGQKT | |
| | | | QRRWFLDKLVDRIGVGHVRDSGSMSEYYLSEIKPLHNFLTQLQPFLKL | |
| | | | KQKQANLALKIIEQLPSAKESPDKFLEVVTWVDQVAALNDSKTRKTTS | |
| | | | ETVRAVLDSLSEKKKSSP | |
| SCOH-ro34-b11-A | 7E32T33C38H | 8K19S32H33H | MANTKYNEEFLLYLAGFVDGDGSIIAQIKPNQTCKFKHHLSLTFVVTQ | 70 |
| (pCLS3487) | 44V54S68Y70 | 38A44S46G59 | KTQRRWSLDKLVDEIGVGYVYDSGSVSRYVLSEIKPLHNFLTQLQPFL | |
| | S75R77V96E | A61R70S73M | ELKQKQANLVLKIIEQLPSAKESPDKFLEVCTWVDQIAALNDSKTRKT | |
| | | 75E77C80G | TSETVRAVLDSLSEKKKSSPAAGGSDKYNQALSKYNQALSKYNQALSG | |
| | | | GGGSNKKFLLYLAGFVDSDGSIIAQIKPNQHHKFKHALSLTFVGQKTQ | |
| | | | RRWFLDKLADRIGVGYVRDSGSMSEYCLSGIKPLHNFLTQLQPFLKLK | |
| | | | QKQANLVLKIIEQLPSAKESPDKFLEVCTWVDQIAALNDSKTRKTTSE | |
| | | | TVRAVLDSLSEKKKSSP | |
| SCOH-ro34-b11-C | 7E32T33C38H | 8K19S32H33H | MANTKYNEEFLLYLAGFVDGDGSIIAQIKPNQTCKFKHHLSLTFVVTQ | 71 |
| (pCLS3488) | 44V54S68Y70 | 38A44S46G59 | KTQRRWSLDKLVDEIGVGYVYDSGSVSRYVLSEIKPLHNFLTQLQPFL | |
| | S75R77V96E1 | A61R70S73M | ELKQKQANLVLKIIEQLPSAKESPDKFLEVCTWVDQVAALNDSKTRKT | |
| | 32V | 75E77C80G13 | TSETVRAVLDSLSEKKKSSPAAGGSDKYNQALSKYNQALSKYNQALSG | |
| | | 2V | GGGSNKKFLLYLAGFVDSDGSIIAQIKPNQHHKFKHALSLTFSVGQKT | |
| | | | QRRWFLDKLADRIGVGYVRDSGSMSEYCLSGIKPLHNFLTQLQPFLKL | |
| | | | KQKQANLVLKIIEQLPSAKESPDKFLEVCTWVDQVAALNDSKTRKTTS | |
| | | | ETVRAVLDSLSEKKKSSP | |
| SCOH-ro34-b11-E | 7E32T33C38H | 8K19S32H33H | MANTKYNEEFLLYLAGFVDGDGSIIAQIKPNQTCKFKHHLSLTFVVTQ | 72 |
| (pCLS3489) | 44V54S68Y70 | 38A44S46G59 | KTQRRWSLDKLVDEIGVGYVYDSGSVSRYVLSKIKPLHNFLTQLQPFL | |
| | S75R77V80K9 | A61R70S73M | ELKQKQANLVLKIIEQLPSAKESPDKFLEVCTWVDQVAALNDSKTRKT | |
| | 6E132V | 75E77C80G10 | TSETVRAVLDSLSEKKKSSPAAGGSDKYNQALSKYNQALSKYNQALSG | |
| | | 5A132V | GGGSNKKFLLYLAGFVDSDGSIIAQIKPNQHHKFKHALSLTFSVGQKT | |
| | | | QRRWFLDKLADRIGVGYVRDSGSMSEYCLSGIKPLHNFLTQLQPFLKL | |
| | | | KQKQANLALKIIEQLPSAKESPDKFLEVCTWVDQVAALNDSKTRKTTS | |
| | | | ETVRAVLDSLSEKKKSSP | |
| SCOH-ro34-b12-A | 7E32T33C38H | 8K19S32H33H | MANTKYNEEFLLYLAGFVDGDGSIIAQIKPNQTCKFKHHLSLTFVVTQ | 73 |
| (pCLS3490) | 44V54S68Y70 | 38A44S46G61 | KTQRRWSLDKLVDEIGVGYVYDSGSVSRYVLSEIKPLHNFLTQLQPFL | |
| | S75R77V96E | R66H70S73M | ELKQKQANLVLKIIEQLPSAKESPDKFLEVCTWVDQIAALNDSKTRKT | |
| | | 75E77Y105A | TSETVRAVLDSLSEKKKSSPAAGGSDKYNQALSKYNQALSKYNQALSG | |
| | | | GGGSNKKFLLYLAGFVDSDGSIIAQIKPNQHHKFKHALSLTFSVGQKT | |
| | | | QRRWFLDKLVDRIGVGHVRDSGSMSEYYLSEIKPLHNFLTQLQPFLKL | |
| | | | KQKQANLALKIIEQLPSAKESPDKFLEVCTWVDQIAALNDSKTRKTTS | |
| | | | ETVRAVLDSLSEKKKSSP | |
| SCOH-ro34-b56- | 7E32T33C38H | 8K19S32H33H | MANTKYNEEFLLYLAGFVDGDGSIIAQIKPNQTCKFKHHLSSTFVVTQ | 74 |
| C_V2 | 41S44V68Y77 | 38A44S46G61 | KTQRRWFLDKLVDEIGVGYVYDRGSVSDYVLSEIKPLHNFLTQLQPFL | |
| (pCLS4321) | V96E132V | R66H70S73M | ELKQKQANLVLKIIEQLPSAKESPDKFLEVCTWVDQVAALNDSKTRKT | |
| | | 75E77Y105A1 | TSETVRAVLDSLSEKKKSSPAAGGSDKYNQALSKYNQALSKYNQALSG | |
| | | 32V | GGGSNKKFLLYLAGFVDSDGSIIAQIKPNQHHKFKHALSLTFSVGQKT | |
| | | | QRRWFLDKLVDRIGVGHVRDSGSMSEYYLSEIKPLHNFLTQLQPFLKL | |
| | | | KQKQANLALKIIEQLPSAKESPDKFLEVCTWVDQVAALNDSKTRKTTS | |
| | | | ETVRAVLDSLSEKKKSSP | |

### Example 2

### Engineering Meganucleases Targeting the Rho_{—}7 Locus

Rho_{—}7 is a locus comprising a 24 bp non-palindromic target (GTCAGCCACCACACAGAAGGCAGA also referred to as Rho_{—}7.1 target=SEQ ID NO: 20) that is present in the exon4 of RHO gene (reference sequence NC000003.11 as described in 10062009 database update; start 3915 bp-3938 bp, downstream of the ATG.

Rho-7 being located in Exon 4, this locus can be used for strategies such as:
gene correction or gene modification (cell line engineering at Rho_{—}7 locus with reporter genes for example) in the presence of a repair matrix.
introduction of a functional cds to follow a exon KI strategy especially well suited for proximal and downstream (3′) mutations.

I-CreI heterodimers able to cleave Rho_{—}7.1 target sequence (SEQ ID NO: 20) were identified using methods derived from those described in Chames et al. (Nucleic Acids Res., 2005, 33, e178), Arnould et al. (J. Mol. Biol., 2006, 355, 443-458), Smith et al. (Nucleic Acids Res., 2006, 34, e149), Arnould et al. (Arnould et al. J Mol. Biol. 2007 371:49-65). Active heterodimers on Rho_{—}7.1 target (=SEQ ID NO: 20) were identified in Yeast. These results were then utilized to design single-chain meganucleases directed against the target sequence SEQ ID NO: 20. These single-chain meganucleases were cloned into mammalian expression vectors and tested for Rho_{—}7.1 cleavage in CHO cells. Strong cleavage activity of the Rho_{—}7.1 target could be observed for these single chain molecules in mammalian cells.

### Example 2.1

### Identification of Meganucleases Cleaving Rho_{—}7

I-CreI variants potentially cleaving Rho_{—}7.1 target sequence in heterodimeric form were constructed by genetic engineering. Pairs of such variants were then co-expressed in yeast. Upon co-expression, one obtains three molecular species, namely two homodimers and one heterodimer. It was then determined whether the heterodimers were capable of cutting the Rho_{—}7.1 target sequence of SEQ ID NO: 20.

a) Construction of Variants of the I-CreI Meganuclease Cleaving Palindromic Sequences Derived from the Rho_{—}7.1 Target Sequence

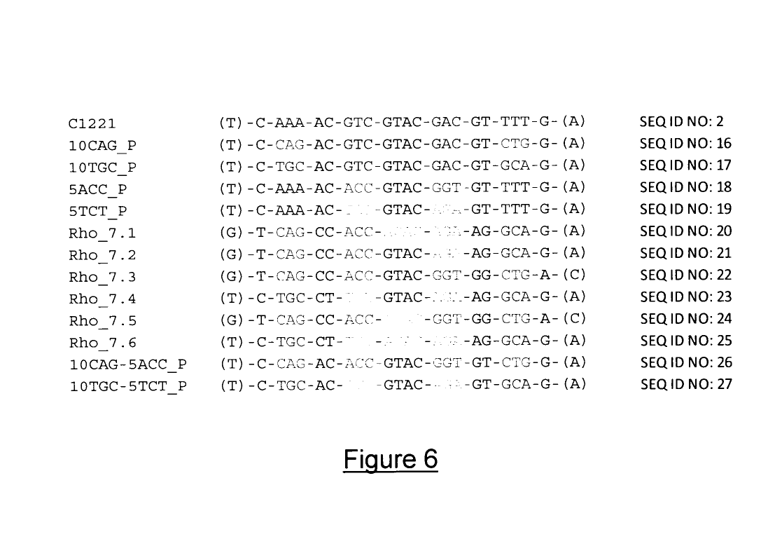

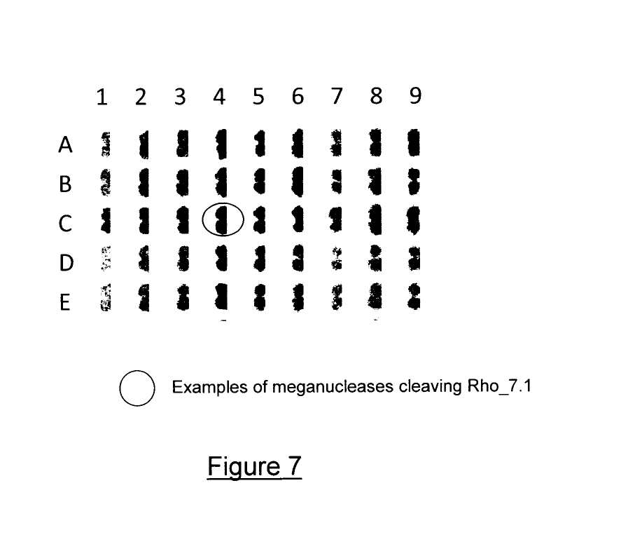

The Rho_{—}7.1 sequence is partially a combination of the 10CAG_P (SEQ ID NO: 16), 5ACC_P (SEQ ID NO: 18), 10TGC_P (SEQ ID NO: 17) and 5TCT_P (SEQ ID NO: 19) target sequences which are shown on FIG. 6. These sequences are cleaved by mega-nucleases obtained as described in International PCT applications WO 2006/097784 and WO 2006/097853, Arnould et al. (J. Mol. Biol., 2006, 355, 443-458) and Smith et al. (Nucleic Acids Res., 2006). Thus, Rho_{—}7.1 should be cleaved by combinatorial variants resulting from these previously identified meganucleases.

A series of targets were derived from Rho_{—}7.1 (FIG. 6). The palindromic targets, Rho_{—}7.5 (GTCAGCCACCACACGGTGGCTGAC=SEQ ID NO: 24) and Rho_{—}7.6 (TCTGCCTTCTACACAGAAGGCAGA=SEQ ID NO: 25), should be cleaved by homodimeric proteins. Therefore, homodimeric I-CreI variants cleaving either the Rho_{—}7.5 palindromic target sequence of SEQ ID NO: 24 or the Rho_{—}7.6 palindromic target sequence of SEQ ID NO: 25 were constructed using methods derived from those described in Chames et al. (Nucleic Acids Res., 2005, 33, e178), Arnould et al. (J. Mol. Biol., 2006, 355, 443-458), Smith et al. (Nucleic Acids Res., 2006, 34, e149) and Arnould et al. (Arnould et al. J Mol. Biol. 2007 371:49-65).

b) Construction of Target Vector

An oligonucleotide of SEQ ID NO: 79, corresponding to the Rho_{—}7.1 target sequence flanked by gateway cloning sequences, was ordered from PROLIGO. This oligo has the following sequence:

TGGCATACAAGTTTGTCAGCCACCACACAGAAGGCAGACAATCGTCTG
TCA.

Double-stranded target DNA, generated by PCR amplification of the single stranded oligonucleotide, was cloned into the pCLS1055 yeast reporter vector using the Gateway protocol (INVITROGEN).

Yeast reporter vector was transformed into the FYBL2-7B Saccharomyces cerevisiae strain having the following genotype: MAT a, ura3Δ851, trp1Δ63, leu2Δ1, lys2Δ202. The resulting strain corresponds to a reporter strain (MILLEGEN).

c) Co-Expression of Variants

The open reading frames coding for the variants cleaving the Rho_{—}7.5 or the Rho_{—}7.6 sequences were cloned into the pCLS542 and pCLS1107 expression vectors, respectively. Yeast DNA from these variants was extracted using standard protocols and was used to transform E. coli. The resulting plasmids were then used to co-transform yeast. Transformants were selected on synthetic medium lacking leucine and containing G418.

d) Mating of Meganucleases Coexpressing Clones and Screening in Yeast

Mating was performed using a colony gridder (QpixII, Genetix). Variants were gridded on nylon filters covering YPD plates, using a low gridding density (4-6 spots/cm²). A second gridding process was performed on the same filters to spot a second layer consisting of different reporter-harboring yeast strains for each target. Membranes were placed on solid agar YPD rich medium, and incubated at 30° C. for one night, to allow mating. Next, filters were transferred to synthetic medium, lacking leucine and tryptophan, adding G418, with galactose (2%) as a carbon source, and incubated for five days at 37° C., to select for diploids carrying the expression and target vectors. After 5 days, filters were placed on solid agarose medium with 0.02% X-Gal in 0.5 M sodium phosphate buffer, pH 7.0, 0.1% SDS, 6% dimethyl formamide (DMF), 7 mM β-mercaptoethanol, 1% agarose, and incubated at 37° C., to monitor β-galactosidase activity. Results were analyzed by scanning and quantification was performed using an appropriate software.

e) Results

Co-expression of different variants resulted in cleavage of the Rho_{—}7.1 target in all of the 36 tested combinations are summarized in Table III herebelow. In this table, “+” indicates a functional combination on the Rho_{—}7 target sequence, i.e., the heterodimer is able to cleave the Rho_{—}7 target sequence. SEQ ID NO: 62 to 65 correspond to variants cleaving Rho34.5 target (SEQ ID NO: 24). SEQ ID NO: 53 to 61 correspond to variants cleaving Rho34.6 target (SEQ ID NO: 25).

TABLE III
I-CreI variants cleaving the Rho_7.5 and Rho_7.6 targets.

| | | Amino acids positions and residues of the I-CreI variants | | | | |
|---|---|---|---|---|---|---|
| | | cleaving the Rho_7.6 target (SEQ ID NO: 25) | | | | |
| | | 17A28S33 | 28S33S38 | 28S33S38 | 28S33S38 | 28S33S38 |
| | | S38R40R5 | R40R54L6 | R40R54L6 | R40R54L6 | R40R54L5 |
| | | 4L68S70S | 8S70S75N | 8S70S75N | 8S70S75N | 9L68S70S7 |
| | | 75N77R82 | 77R82E87 | 77R82E142 | 77R82E89 | 5N77R82E |
| | | E151A | L125A131R | R151A | A131R | 131R |
| | | (SEQ ID | (SEQ ID | (SEQ ID | (SEQ ID | (SEQ ID |
| | | NO: 53) | NO: 54) | NO: 55) | NO: 56) | NO: 57) |
| Amino | 1T9L33N3 | + | + | + | + | + |
| acids | 8Y40R43L | | | | | |
| positions | 44K54L57 | | | | | |
| and | E68Y70S7 | | | | | |
| resdidues | 5Y77Q85R | | | | | |
| of the | 86S89A15 | | | | | |
| I-CreI | 4T | | | | | |
| variants | (SEQ ID | | | | | |
| cleaving | NO: 62) | | | | | |
| the | 9L33N38Y | + | + | + | + | + |
| Rho_7.5 | 40R43L44 | | | | | |
| target | K54L57E6 | | | | | |
| (SEQ | 8Y70S75Y | | | | | |
| ID | 77Q8SR86 | | | | | |
| NO: 24) | S89A158E | | | | | |
| | (SEQ ID | | | | | |
| | NO: 63) | | | | | |
| | 9L24V33N | + | + | + | + | + |
| | 38Y40R43 | | | | | |
| | L44K54L5 | | | | | |
| | 7E68Y70S | | | | | |
| | 75Y77Q85 | | | | | |
| | R86S89A1 | | | | | |
| | 56G | | | | | |
| | (SEQ ID | | | | | |
| | NO: 64) | | | | | |
| | 9L33S38Y | + | + | + | + | + |
| | 40R43L44 | | | | | |
| | K54L57E6 | | | | | |
| | 8Y70S75Y | | | | | |
| | 77Q86S89 | | | | | |
| | A149H | | | | | |
| | (SEQ ID | | | | | |
| | NO: 65) | | | | | |

| | | Amino acids positions and residues of the I-CreI variants | | | |
|---|---|---|---|---|---|
| | | cleaving the Rho_7.6 target (SEQ ID NO: 25) | | | |
| | | | | 28S33S38 | |
| | | 28S33S38 | 28S33S38 | R40R54L6 | 28S33S38 |
| | | R40R54L6 | R40R54L6 | 8S70S75N | R40R54L6 |
| | | 8S70S75N | 8S70S75N | 77R82E151 | 8S70S75N |
| | | 77R82E131R | 77R82E | A164T | 77R82E151A |
| | | (SEQ ID | (SEQ ID | (SEQ ID | (SEQ ID |
| | | NO: 58) | NO: 59) | NO: 60) | NO: 61) |
| Amino | 1T9L33N3 | + | + | + | + |
| acids | 8Y40R43L | | | | |
| positions | 44K54L57 | | | | |
| and | E68Y70S7 | | | | |
| resdidues | 5Y77Q85R | | | | |
| of the | 86S89A15 | | | | |
| I-CreI | 4T | | | | |
| variants | (SEQ ID | | | | |
| cleaving | NO: 62) | | | | |
| the | 9L33N38Y | + | + | + | + |
| Rho_7.5 | 40R43L44 | | | | |
| target | K54L57E6 | | | | |
| (SEQ | 8Y70S75Y | | | | |
| ID | 77Q8SR86 | | | | |
| NO: 24) | S89A158E | | | | |
| | (SEQ ID | | | | |
| | NO: 63) | | | | |
| | 9L24V33N | + | + | + | + |
| | 38Y40R43 | | | | |
| | L44K54L5 | | | | |
| | 7E68Y70S | | | | |
| | 75Y77Q85 | | | | |
| | R86S89A1 | | | | |
| | 56G | | | | |
| | (SEQ ID | | | | |
| | NO: 64) | | | | |
| | 9L33S38Y | + | + | + | + |
| | 40R43L44 | | | | |
| | K54L57E6 | | | | |
| | 8Y70S75Y | | | | |
| | 77Q86S89 | | | | |
| | A149H | | | | |
| | (SEQ ID | | | | |
| | NO: 65) | | | | |

In conclusion, several heterodimeric I-CreI variants able to cleave Rho_{—}7 target sequence in yeast were identified.

### Example 2.2

### Validation of Rho_{—}7 Target Cleavage in an Extrachromosomal Model in CHO Cells by Covalent Assembly of Heterodimers as Single Chain and Improvement of Meganucleases Cleaving Rho_{—}7

I-CreI variants able to efficiently cleave the Rho_{—}7 target in yeast when forming heterodimers are described hereabove in example 2.1. In order to further assess the cleavage activity for the Rho_{—}7 target in CHO cells, synthetic single chain molecules based on several pairs of mutants identified in Yeast have been assayed using an extrachromosomal assay in CHO cells. The screen in CHO cells is a single-strand annealing (SSA) based assay where cleavage of the target by the meganucleases induces homologous recombination and expression of a LagoZ reporter gene (a derivative of the bacterial lacZ gene).

The M1×MA Rho_{—}7 heterodimer gives high cleavage activity in yeast. Rho_{—}7.5-MA is a Rho_{—}7.5 cutter that bears the following mutations in comparison with the I-CreI wild type sequence: 9L 33S 38Y 40R 43L 44K 54L 57E 68Y 70S 75Y 77Q 86S 89A 149H. Rho_{—}7.6-M1 is a Rho_{—}7.6 cutter that bears the following mutations in comparison with the I-CreI wild type sequence: 17A 28S 33S 38R 40R 54L 68S 70S 75N 77R 82E 151A.

Single chain constructs were engineered using the linker RM2 (AAGGSDKYNQALSKYNQALSKYNQALSGGGGS=SEQ ID NO: 78), thus resulting in the production of the single chain molecule: M1-linkerRM2-MA. During this design step, the G19S mutation was introduced into the C-terminal MA variant. In addition, mutations K7E and K96E were introduced into the M1 variant and mutations E8K and E61R into the MA variant to create the single chain molecule: M1 (K7E K96E)-linkerRM2-MA (E8K E61R G19S) that is further called SCOH-ro7-b1 scaffold. Some additional amino-acid substitutions have been found in previous studies to enhance the activity of I-CreI derivatives: I132V (replacement of Isoleucine 132 with Valine), E80K and V105A are some of these mutations of potential interest. The I132V, E80K and V105A mutations were introduced into either one, both or none of the coding sequence of N-terminal and C-terminal protein fragments as described in table IV.

A similar strategy was applied to a second scaffold, termed SCOH-Ro7-b56 scaffold, based on the other variants cleaving Rho_{—}7.5 (9L 24V 33N 38Y 40R 43L 44K 54L 57E 68Y 70S 75Y 77Q 85R 86S 89A 156G) and Rho_{—}7.6 (28S 33S 38R 40R 54L 59L 68S 70S 75N 77R 82E 131R) as homodimers, respectively. The resulting proteins are shown in Table IV below. All the single chain molecules were assayed in CHO for cleavage of the Rho_{—}7 target.

a) Cloning of Rho_{—}7 Target in a Vector for CHO Screen

An oligonucleotide corresponding to the Rho_{—}7 target sequence flanked by gateway cloning sequences, was ordered from PROLIGO (TGGCATACAAGTTTGTCAGCCACCACACAGAAGGCAGACAATCGTCTGTCA=SEQ ID NO: 79). Double-stranded target DNA, generated by PCR amplification of the single stranded oligonucleotide, was cloned using the Gateway protocol (INVITROGEN) into the pCLS 1058 CHO reporter vector. Cloned target was verified by sequencing (MILLEGEN).

b) Cloning of the Single Chain Molecule

A series of synthetic gene assembly was ordered to MWG-EUROFINS. Synthetic genes coding for the different single chain variants targeting Rho_{—}7 (“SCOH-ro7”) were cloned into pCLS 1853 using AscI and XhoI restriction sites.

c) Extrachromosomal Assay in Mammalian Cells

CHO K1 cells were transfected as described in example 1.2. 72 hours after transfection, culture medium was removed and 150 μl of lysis/revelation buffer for β-galactosidase liquid assay was added. After incubation at 37° C., OD was measured at 420 nm. The entire process was performed on an automated Velocity11 BioCel platform. Per assay, 150 ng of target vector was cotransfected with an increasing quantity of variant DNA from 3.12 to 25 ng (25 ng of single chain DNA corresponding to 12.5 ng+12.5 ng of heterodimer DNA). Finally, the transfected DNA variant DNA quantity was 3.12 ng, 6.25 ng, 12.5 ng and 25 ng. The total amount of transfected DNA was completed to 175 ng (target DNA, variant DNA, carrier DNA) using an empty vector (pCLS0002).

d) Results

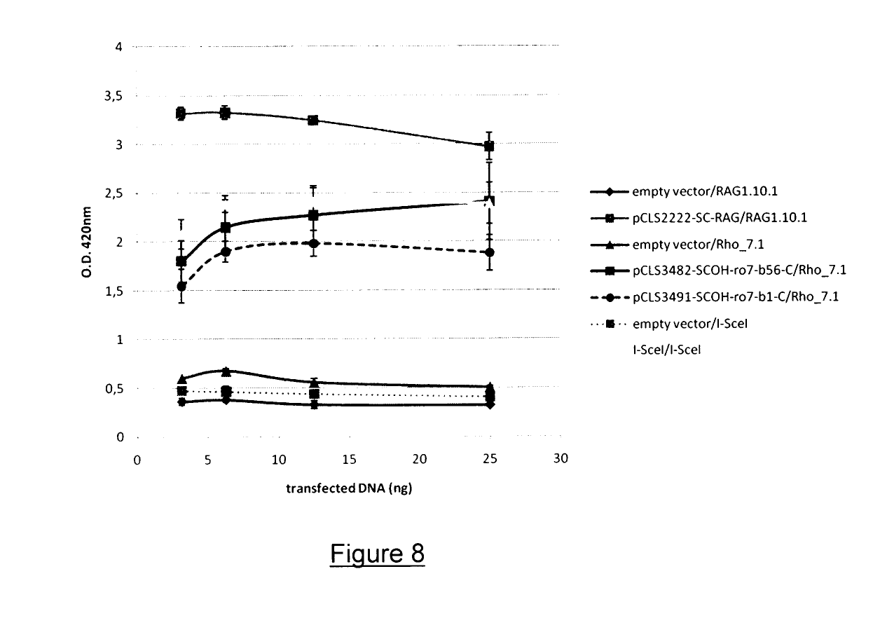

The activity of the single chain molecules against the Rho_{—}7 target was monitored using the previously described CHO assay along with our internal control SCOH-RAG (pCLS2222) and I-Sce I meganucleases. All comparisons were done at 3.12 ng, 6.25 ng, 12.5 ng, and 25 ng transfected variant DNA (FIG. 8). Examples of Single chain molecules displaying Rho_{—}7 target cleavage activity in CHO assay are listed in Table IV below.

TABLE IV
Examples of single chain series designed for strong cleavage
of Rho 7 target in CHO cells

| | Mutations | Mutations | | |
|---|---|---|---|---|
| | on N- | on C- | | |
| | terminal | terminal | | SEQ |
| Name | monomer | monomer | Protein sequence | ID NO: |
| SCOH-ro7-b56-C | 7E28S33S38R4 | 8K9L19S24V33 | MANTKYNEEFLLYLAGFVDGDGSIIAQISPNQSSKFKHRLRLTFQVT | 75 |
| (pCLS3482) | 0R54L59L68S70 | N38Y40R43L44 | QKTQRRWLLDKLLDEIGVGYVSDSGSVSNYRLSEIEPLHNFLTQLQP | |
| | S75N77R82E96 | K54LS7E61R68 | FLELKQKQANLVLKIIEQLPSAKESPDKFLEVCTWVDRVAALNDSKT | |
| | E131R132V | Y70S75Y77Q85 | RKTTSETVRAVLDSLSEKKKSSPAAGGSDKYNQALSKYNQALSKYNQ | |
| | | R86S89A132V1 | ALSGGGGSNKKLLLYLAGFVDSDGSIVAQIKPNQSNKFKHYLRLTLK | |
| | | 56G | VTQKTQRRWLLDELVDRIGVGYVYDSGSVSYYQLSEIKPLRSFLAQL | |
| | | | QPFLKLKQKQANLVLKIIEQLPSAKESPDKFLEVCTWVDQVAALNDS | |
| | | | KTRKTTSETVRAVLDSLGEKKKSSP | |
| SCOH-ro7-b1-C | 7E17A28S33S3 | 8K9L19S33S | MANTKYNEEFLLYLAGFADGDGSIIAQISPNQSSKFKHRLRLTFQVT | 76 |
| (pCLS3491) | 8R40R54L68S7 | 38Y40R43L44 | QKTQRRWLLDKLVDEIGVGYVSDSGSVSNYRLSEIEPLHNFLTQLQP | |
| | 0S75N77R82E9 | K541S7E61R | FLELKQKQANLVLKIIEQLPSAKESPDKFLEVCTWVDQVAALNDSKT | |
| | 6E132V151A | 68Y70S7SY77 | RKTTSETVRAALDSLSEKKKSSPAAGGSDKYNQALSKYNQALSKYNQ | |
| | | Q86S89A132 | ALSGGGGSNKKLLLYLAGFVDSDGSIIAQIKPNQSSKFKHYLRLTLK | |
| | | V149H | VTQKTQRRWLLDELVDRIGVGYVYDSGSVSYYQLSEIKPLHSFLAQL | |
| | | | QPFLKLKQKQANLVLKIIEQLPSAKESPDKFLEVCTWVDQVAALNDS | |
| | | | KTRKTTSETVHAVLDSLSEKKKSSP | |

Variants shared specific behaviour upon assayed dose depending on the mutation profile they bear (FIG. 8). For example, pCLS3482 SCOH-ro7-b56-C displayed a slightly higher activity than pCLS3491 SCOH-ro7-b1-C. Both pCLS3482 and pCLS3491 show activity levels comparable to I-SceI, a molecule of reference in the field of genome engineering.

All of the “SCOH-ro7” variants active in CHO assay can be considered for genome engineering at Rho_{—}7 locus including insertion of transgenes, gene modification, gene correction and mutagenesis.

Numerous modifications and variations on the present invention are possible in light of the above teachings. It is, therefore, to be understood that within the scope of the accompanying claims, the invention may be practiced otherwise than as specifically described herein.

### Example 3

### Engineering Meganucleases Targeting the Rho36 Locus

Rho36 is a locus comprising a 24 bp non-palindromic target (CAGATCCCACTTAACAGAGAGGAA also referred to as Rho36.1 target=SEQ ID NO: 32) that is present in the intron1 of RHO gene (reference sequence NC000003.11 as described in 10062009 database update; start 1177 bp-1200 bp).

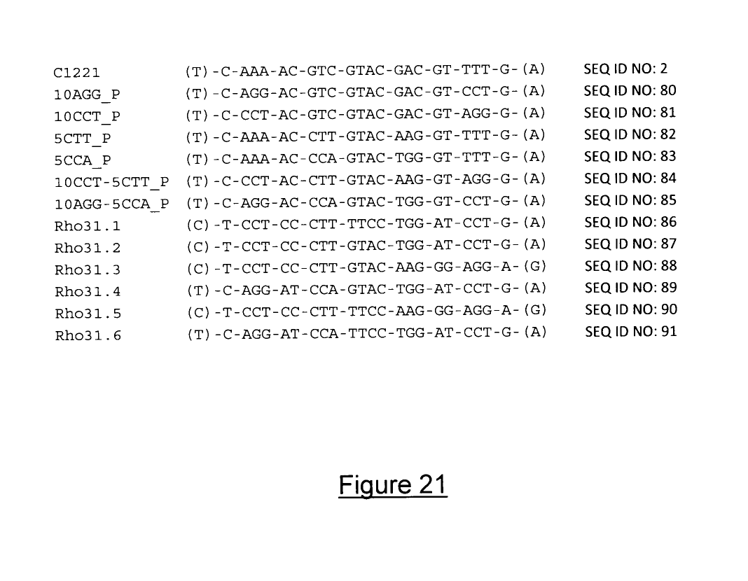

Rho36 being located in an intron, this locus can be used for strategies such as the introduction of a functional cds to follow a exon KI strategy especially well suited for proximal and downstream (3′) mutations.
As previously described in examples 1 and 2, a series of targets were derived from Rho36 (FIG. 21). The palindromic targets, Rho36.5 (SEQ ID NO: 36) and Rho36.6 (SEQ ID NO: 37), should be cleaved by homodimeric proteins. Therefore, homodimeric I-CreI variants cleaving either the Rho36.5 palindromic target sequence of SEQ ID NO: 36 or the Rho36.6 palindromic target sequence of SEQ ID NO: 37 were constructed using methods derived from those described in Chames et al. (Nucleic Acids Res., 2005, 33, e178), Arnould et al. (J. Mol. Biol., 2006, 355, 443-458), Smith et al. (Nucleic Acids Res., 2006, 34, e149) and Arnould et al. (Arnould et al. J Mol. Biol. 2007 371:49-65).
Amino acids positions and residues of I-CreI variants cleaving Rho36.5 and Rho36.6 targets are shown in Tables V and VI below:

TABLE V
I-CreI variants cleaving Rho 36.5 target
Amino acids positions and residues of the I-CreI variants cleaving
the Rho36.5 target (SEQ ID NO: 36)

| | |
|---|---|
| 32G33H44R68Y77W103S | SEQ ID NO: 92 |
| 32G33H44R68Y72P77W105A | SEQ ID NO: 93 |
| 32G33H44R68Y77W105A | SEQ ID NO: 94 |
| 32G33H44R68Y77W | SEQ ID NO: 95 |
| 32G33H44R68Y77W85R | SEQ ID NO: 96 |
| 32G33H44R66H68Y77W109V | SEQ ID NO: 97 |
| 32G33H44R68Y77W116R | SEQ ID NO: 98 |
| 32G33H44R68Y77W121R | SEQ ID NO: 99 |
| 31R32G33H44R68Y77W | SEQ ID NO: 100 |

TABLE VI
I-CreI variants cleaving Rho 36.6 target
Amino acids positions and residues of the I-CreI variants cleaving
the Rho36.6 target (SEQ ID NO: 37)

| | |
|---|---|
| 33S38Y44R57R66H68Y70S75N77T | SEQ ID NO: 101 |
| 33S38Y44R57R66H68Y70S75N77T | SEQ ID NO: 102 |
| 33S38Y44R57R66H68Y70S71R75N77T87L105A | SEQ ID NO: 103 |

I-CreI heterodimers able to cleave Rho36.1 target sequence (SEQ ID NO: 32) were identified using methods derived from those described in Chames et al. (Nucleic Acids Res., 2005, 33, e178), Arnould et al. (J. Mol. Biol., 2006, 355, 443-458), Smith et al. (Nucleic Acids Res., 2006, 34, e149), Arnould et al. (Arnould et al. J Mol. Biol. 2007 371:49-65). With the same methods previously described in examples 1 and 2, active heterodimers on Rho36.1 target (SEQ ID NO: 32) were identified in Yeast. Some active heterodimers are listed in table VII below.

TABLE VII
Active heterodimers cleaving Rho 36.1 target

| Amino acids positions and residues of the | | |
|---|---|---|
| I-CreI variants cleaving the Rho36.5 | Amino acids positions and residues of the | Activity |
| target | I-CreI variants cleaving the Rho36.6 target | in Yeast |
| 32G33H44R68Y77W121R | 33S38Y44R57R66H68Y70S75N77T | + |
| (SEQID NO: 99) | (SEQID NO: 101) | |
| 31R32G33H44R68Y77W | 33S38Y44R57R66H68Y70S75N77T | + |
| (SEQ ID NO: 100) | (SEQ ID NO: 101) | |
| 32G33H44R68Y77W121R | 33S38Y44R57R66H68Y70S75N77T | + |
| (SEQ ID NO: 99) | (SEQ ID NO: 102) | |
| 31R32G33H44R68Y77W | 33S38Y44R57R66H68Y70S75N77T | + |
| (SEQ ID NO: 100) | (SEQID NO: 102) | |
| 32G33H44R68Y77W103S | 33S38Y44R57R66H68Y70S71R75N77T87L | + |
| (SEQ ID NO: 92) | 105A | |
| | (SEQ ID NO: 103) | |
| 32G33H44R68Y72P77W105A | 33S38Y44R57R66H68Y70S71R75N77T87 | + |
| (SEQ ID NO: 93) | L105A | |
| | (SEQ ID NO: 103) | |
| 32G33H44R68Y77W105A | 33S38Y44R57R66H68Y70S71R75N77T87L | + |
| (SEQ ID NO: 94) | 105A | |
| | (SEQ ID NO: 103) | |
| 32G33H44R68Y77W | 33S38Y44R57R66H68Y70S71R75N77T87L | + |
| (SEQ ID NO: 95) | 105A | |
| | (SEQ ID NO: 103) | |
| 32G33H44R68Y77W85R | 33S38Y44R57R66H68Y70S71R75N77T87L | + |
| (SEQ ID NO: 96) | 105A | |
| | (SEQ ID NO: 103) | |
| 32G33H44R66H68Y77W109V | 33S38Y44R57R66H68Y70S71R75N77T87L | + |
| (SEQ ID NO: 97) | 105A | |
| | (SEQ ID NO: 103) | |
| 32G33H44R68Y77W116R | 33S38Y44R57R66H68Y70S71R75N77T87L | + |
| (SEQ ID NO: 98) | 105A | |
| | (SEQ ID NO: 103) | |
| 31R32G33H44R68Y77W | 33S38Y44R57R66H68Y70S71R75N77T87L | + |
| (SEQ ID NO: 100) | 105A | |
| | (SEQ ID NO: 103) | |

These results were then utilized to design single-chain meganucleases directed against the Rho36.1 target sequence (SEQ ID NO: 32). The heterodimer providing the best cleavage activity (in bold in Table VII) has been used to design a single chain molecule. The M1×MA Rho36 heterodimer gives high cleavage activity in yeast. Rho36.5-M1 is a Rho36.5 cutter that bears the mutations 32G 33H 44R 68Y 72P 77W 105A (SEQ ID NO: 93) when compared to I-CreI wild type sequence. Rho36.6-MA is a Rho36.6 cutter that bears the mutations 33S 38Y 44R 57R 66H 68Y 70S 71R 75N 77T 87L 105A 33S38Y44R57R66H68Y70S71R75N77T87L105A (SEQ ID NO: 103) when compared to I-CreI wild type sequence. Single chain constructs were engineered using the linker RM2(AAGGSDKYNQALSKYNQALSKYNQALSGGGGS=SEQ ID NO: 78), thus resulting in the production of the single chain molecule: M1-linkerRM2-MA. During this design step, the G19S mutation was introduced into the C-terminal MA variant. In addition, mutations K7E and K96E were introduced into the M1 variant and mutations E8K and E61R into the MA variant to create the single chain molecule: M1 (K7E K96E)-linkerRM2-MA (E8K E61R G19S) that is further called SCOH-Ro36-b1-C scaffold. Some additional amino-acid substitutions have been found in previous studies to enhance the activity of I-CreI derivatives such as I132V (replacement of Isoleucine 132 with Valine), E80K and V105A. The I132V, E80K and V105A mutations were introduced into either one, both or none of the coding sequence of N-terminal and C-terminal protein fragments as described in the following table VIII. The single chain construct described below has been designed and cloned into yeast and mammalian expression vectors but any active heterodimer pair could be used to generate alternative scaffolds.

TABLE VIII
Single chain designed for Rho36 target

| | Mutations on N-terminal | | SEQ ID |
|---|---|---|---|
| Name | monomer | Mutations on C-terminal monomer | No. |
| SCOH-Ro36-b1-C | 7E32G33H44R68Y72P77W96E1 | 8K19S33S38Y44R57R61R66H68Y70 | 104 |
| (pCLS5645) | 05A132V | S71R75N77T87L105A132V | |

The single chain molecule designed based on heterodimer cleavage of Rho36.1 can be considered for genome engineering at Rho36 locus including insertion of transgenes, gene modification and gene correction.

### Example 4

### Engineering Meganucleases Targeting the Rho31 Locus

Rho31 is a locus comprising a 24 bp non-palindromic target (CTCCTCCCTTTTCCTGGATCCTGA also referred to as Rho31.1 target=SEQ ID NO: 86) that is present in the region upstream of the 1^{st} exon of Rho gene that will be referred to as “preExon1”. Rho31 locus can be located precisely on whole genome assembly as displayed in the table IX below also recapitulating the targets described in previous examples:

TABLE IX
Genomic positions of Rho targets (Y for yes; N or no)

| | | | | | | | In | | | |
|---|---|---|---|---|---|---|---|---|---|---|
| | | | Position on | | Position | In | Promoter | In | In | exon |
| name | site_sequence | chromosome | chromosome | contig | in contig | Gene | Region | CDS | Exon | −n° |
| Rho3 | CTCCTCCCTTTTCCTGGATCCTGA | 3 | 129247268 | NT_005 | 35742414 | N | RHO, | | | |
| 1.1 | | | | 612 | | | | | | |
| Rho3 | ACTTCCTCACGCTCTACGTCACCG | 3 | 129247740 | NT_005 | 35742886 | Y | N | Y | Y | 1 |
| 4.1 | | | | 612 | | | | | | |
| Rho3 | CAGATCCCACTTAACAGAGAGGAA | 3 | 129248658 | NT_005 | 35743804 | Y | N | N | N | — |
| 6.1 | | | | 612 | | | | | | |
| Rho_ | GTCAGCCACCACACAGAAGGCAGA | 3 | 129251396 | NT_005 | 35746542 | Y | N | Y | Y | 4 |
| 7.1 | | | | 612 | | | | | | |

Rho31 being located in the preExon1, this locus can be used for strategies sum as:
gene correction or gene modification (cell line engineering at Rho31 locus with reporter genes for example) in the presence of a repair matrix.
introduction of a functional cds to follow a exon KI strategy especially well suited for proximal and downstream (3′) mutations.
Amongst all possible gene modifications that can be attempted, it can be used to engineer RHO promoter.
   As described in previous examples, a series of targets were derived from Rho31 (FIG. 21). The palindromic targets, Rho31.5 (SEQ ID NO: 90) and Rho31.6 (SEQ ID NO: 91), should be cleaved by homodimeric proteins. Therefore, homodimeric I-CreI variants cleaving either the Rho31.5 palindromic target sequence of SEQ ID NO: 90 or the Rho31.6 palindromic target sequence of SEQ ID NO: 91 were constructed using methods derived from those described in Chames et al. (Nucleic Acids Res., 2005, 33, e178), Arnould et al. (J. Mol. Biol., 2006, 355, 443-458), Smith et al. (Nucleic Acids Res., 2006, 34, e149) and Arnould et al. (Arnould et al. J Mol. Biol. 2007 371:49-65).
   Amino acids positions and residues of I-CreI variants cleaving Rho36.5 and Rho36.6 targets are shown in Tables X and XI below:

TABLE X
I-CreI variants cleaving Rho 31.5 target
Amino acids positions and residues of the I-CreI variants cleaving
the Rho31.5 target (SEQ ID NO: 90)

| | |
|---|---|
| 4R8G33S38Y44R68Y70S77N87L105A160R161P | SEQ ID NO: 105 |
| 33S38Y44R68Y70S77N85R87L161P | SEQ ID NO: 106 |
| 33S38Y44R66H68Y70S77N89A157G158E | SEQ ID NO: 107 |
| 33S38Y44R68Y70S77N87L120G161P | SEQ ID NO: 108 |
| 33S38Y44R66H68Y70S77N87L94L157G | SEQ ID NO: 109 |
| 6S33S38Y44R66H68Y70S77N89A157G161P | SEQ ID NO: 110 |
| 33S38Y44R68Y70T77N87L153V161P | SEQ ID NO: 111 |
| 33S38Y44R68Y70S77N87L129M161P | SEQ ID NO: 112 |
| 6S33S38Y44R66H68Y70S77N89A157G | SEQ ID NO: 113 |

TABLE XI
I-CreI variants cleaving Rho 31.6 target
Amino acids positions and residues of the I-CreI variants cleaving
the Rho31.6 target (SEQ ID NO: 91)

| | |
|---|---|
| 28E38R40K43L44K54L70E75N81V96R153V160G | SEQ ID NO: 114 |
| 2I28E38R40K43L44K54L70E75N81V96R153V160R | SEQ ID NO: 115 |
| 33S38Y43L44R68Y70S77N87L161P | SEQ ID NO: 116 |

I-CreI heterodimers able to cleave Rho31.1 target sequence (SEQ ID NO: 86) were identified using methods derived from those described in Chames et al. (Nucleic Acids Res., 2005, 33, e178), Arnould et al. (J. Mol. Biol., 2006, 355, 443-458), Smith et al. (Nucleic Acids Res., 2006, 34, e149), Arnould et al. (Arnould et al. J Mol. Biol. 2007 371:49-65). Some active heterodimers on Rho31.1 target (SEQ ID NO: 86) were identified in Yeast.

TABLE XII
Active heterodimers cleaving Rho 31.1 target

| Amino acids positions and residues of the | | |
|---|---|---|
| I-CreI variants cleaving the Rho31.5 | Amino acids positions and residues of the | Activity |
| target | I-CreI variants cleaving the Rho31.6 target | in Yeast |
| 4R8G33S38Y44R68Y70S77N87L105A | 28E38R40K43L44K54L70E75N81V96R | + |
| 160R161P | 153V160G | |
| (SEQ ID NO: 105) | (SEQ ID NO: 114) | |
| 33S38Y44R68Y70S77N85R87L161P | 28E38R40K43L44K54L70E75N81V96R | + |
| (SEQ ID NO: 106) | 153V160G | |
| | (SEQ ID NO: 114) | |
| 33S38Y44R66H68Y70S77N89A157G158E | 28E38R40K43L44K54L70E75N81V96R | + |
| (SEQ ID NO: 107) | 153V160G | |
| | (SEQ ID NO: 114) | |
| 33S38Y44R68Y70S77N87L120G161P | 28E38R40K43L44K54L70E75N81V96R | + |
| (SEQ ID NO: 108) | 153V160G | |
| | (SEQ ID NO: 114) | |
| 33S38Y44R66H68Y70S77N87L94L157G | 28E38R40K43L44K54L70E75N81V96R | + |
| (SEQ ID NO: 109) | 153V160G | |
| | (SEQ ID NO: 114) | |
| 6S33S38Y44R66H68Y70S77N89A157G | 28E38R40K43L44K54L70E75N81V96R | + |
| 161P | 153V160G | |
| (SEQ ID NO: 110) | (SEQ ID NO: 114) | |
| 33S38Y44R68Y70T77N87L153V161P | 28E38R40K43L44K54L70E75N81V96R | + |
| (SEQ ID NO: 111) | 153V160G | |
| | (SEQ ID NO: 114) | |
| 33S38Y44R68Y70S77N87L129M161P | 28E38R40K43L44K54L70E75N81V96R | + |
| (SEQ ID NO: 112) | 153V160G | |
| | (SEQ ID NO: 114) | |
| 6S33S38Y44R66H68Y70S77N89A157G | 28E38R40K43L44K54L70E75N81V96R | + |
| (SEQ ID NO: 113) | 153V160G | |
| | (SEQ ID NO: 114) | |
| 4R8G33S38Y44R68Y70S77N87L105A | 2I28E38R40K43L44K54L70E75N81V96R | + |
| 160R161P | 153V160R | |
| (SEQ ID NO: 105) | (SEQ ID NO: 115) | |
| 33S38Y44R68Y70S77N85R87L161P | 2I28E38R40K43L44K54L70E75N81V96R | + |
| (SEQ ID NO: 106) | 153V160R | |
| | (SEQ ID NO: 115) | |
| 33S38Y44R66H68Y70S77N89A157G158E | 2I28E38R40K43L44K54L70E75N81V96R | + |
| (SEQ ID NO: 107) | 153V160R | |
| | (SEQ ID NO: 115) | |
| 33S38Y44R68Y70S77N87L120G161P | 2I28E38R40K43L44K54L70E75N81V96R | + |
| (SEQ ID NO: 108) | 153V160R | |
| | (SEQ ID NO: 115) | |
| 33S38Y44R66H68Y70S77N87L94L157G | 2I28E38R40K43L44K54L70E75N81V96R | + |
| (SEQ ID NO: 109) | 153V160R | |
| | (SEQ ID NO: 115) | |
| 6S33S38Y44R66H68Y70S77N89A157G | 2I28E38R40K43L44K54L70E75N81V96R | + |
| 161P | 153V160R | |
| (SEQ ID NO: 110) | (SEQ ID NO: 115) | |
| 33S38Y44R68Y70T77N87L153V161P | 2I28E38R40K43L44K54L70E75N81V96R | + |
| (SEQ ID NO: 111) | 153V160R | |
| | (SEQ ID NO: 115) | |
| 33S38Y44R68Y70S77N87L129M161P | 2I28E38R40K43L44K54L70E75N81V96R | + |
| (SEQ ID NO: 112) | 153V160R | |
| | (SEQ ID NO: 115) | |
| 6S33S38Y44R66H68Y70S77N89A157G | 2I28E38R40K43L44K54L70E75N81V96R | + |
| (SEQ ID NO: 113) | 153V160R | |
| | (SEQ ID NO: 115) | |
| 33S38Y44R66H68Y70S77N87L94L157G | 33S38Y43L44R68Y70S77N87L161P | + |
| (SEQ ID NO: 109) | (SEQ ID NO: 116) | |

These results as well as previous homodimer cleavage activity results were then utilized to design a series of single-chain meganucleases directed against the target sequence Rho31.1 (SEQ ID NO: 86) named scaffolds SCOH-Ro31-b1 or SCOH-Ro31-b56 respectively.

The M1×MA Rho31 heterodimer provides the best cleavage activity in yeast. Rho31.5-M1 (SEQ ID NO: 109) is a Rho31.5 cutter that bears the mutations 33S 38Y 44R 66H 68Y 70S 77N 87L 94L 157G when compared to I-CreI wild type sequence. Rho31.6-MA (SEQ ID NO: 114) is a Rho31.6 cutter that bears the mutations 28E 38R 40K 43L 44K 54L 70E 75N 81V 96R 153V 160G. when compared to I-CreI wild type sequence. Single chain constructs were engineered using the linker RM2 (AAGGSDKYNQALSKYNQALSKYNQALSGGGGS; SEQ ID NO: 78), thus resulting in the production of the single chain molecule: M1-linkerRM2-MA. During this design step, the G19S mutation was introduced into the C-terminal MA variant. In addition, mutations K7E and K96E were introduced into the M1 variant and mutations E8K and E61R into the MA variant to create the single chain molecule: M1 (K7E K96E)-linkerRM2-MA (E8K E61R G19S) that is further called SCOH-Ro31-b1 scaffold.

Alternatively the mutants displaying the best activity on Rho31.5 and Rho31.6 as homodimers, also displaying cleavage activity as heterodimers on Rho31.1 have been used to design a series of single chain molecules. The M2×M2 Rho31 heterodimer provides cleavage activity in yeast. Rho36.5-M2 (SEQ ID NO: 106) is a Rho36.5 cutter, displaying highest activity on Rho31.5 as homodimer, that bears the mutations 33S 38Y 44R 68Y 70S 77N 85R 87L 161P when compared to I-CreI wild type sequence. Rho36.6-MB (SEQ ID NO: 115) is a Rho36.6 cutter, displaying highest activity on Rho31.6 as homodimer, that bears the mutations 2I 28E 38R 40K 43L 44K 54L 70E 75N 81V 96R 153V 160R when compared to I-CreI wild type sequence. Single chain constructs were engineered using the linker RM2 (AAGGSDKYNQALSKYNQALSKYNQALSGGGGS; SEQ ID NO: 78), thus resulting in the production of the single chain molecule: M2-linkerRM2-MB. During this design step, the G19S mutation was introduced into the C-terminal MA variant. In addition, mutations K7E and K96E were introduced into the M1 variant and mutations E8K and E61R into the MA variant to create the single chain molecule: M1 (K7E K96E)-linkerRM2-MA (E8K E61R G19S) that is further called SCOH-Ro31-b56 scaffold. Some additional amino-acid substitutions have been found in previous studies to enhance the activity of I-CreI derivatives such as I132V (replacement of Isoleucine 132 with Valine), E80K and V105A. The I132V, E80K and V105A mutations were introduced or not into either one or both coding sequences of N-terminal and C-terminal protein fragments as described in the following table. The mutation 2I was not kept in the single chain molecule as this position is not conserved due to the presence of the linker. Any active heterodimer might be used to generate alternative scaffolds.

These single-chain meganucleases were cloned into both yeast and mammalian expression vectors. Some variant were then tested for Rho31.1 (SEQ ID NO: 86) cleavage in Yeast. Cleavage activity of the Rho31.1 target could be observed for several of these single chain molecules, listed in the table XIII below:

TABLE XIII
Single chains designed for Rho31 target

| | Mutations | Mutations | | | |
|---|---|---|---|---|---|
| | on N- | on C- | | | Cleavage |
| | terminal | terminal | | SEQ | of Rho31.1 |
| Name | monomer | monomer | Protein sequence | ID NO: | in Yeast |
| SCOH-ro31- | 7E33S38Y | 8K19S28E | MANTKYNEEFLLYLAGFVDGDGSIIAQIKPNQSSKFKHYLSLTFRV | 117 | Nd |
| b56-A | 44R68Y70 | 38R40K43 | TQKTQRRWFLDKLVDEIGVGYVYDSGSVSDYNLSEIKPLRNLLTQL | | |
| (pCLS6298) | S77N85R | L44K54L | QPFLELKQKQANLVLKIIEQLPSAKESPDKFLEVCTWVDQIAALND | | |
| | 87L96E16 | 61R70E75 | SKTRKTTSETVRAVLDSLSEKKKPSPAAGGSDKYNQALSKYNQALS | | |
| | 1P | N81V96R | KYNQALSGGGGSNKKFLLYLAGFVDSDGSIIAQIEPNQSYKFKHRL | | |
| | | 153V160R | KLTLKVTQKTQRRWLLDKLVDRIGVGYVRDEGSVSNYILSEVKPLH | | |
| | | | NFLTQLQPFLRLKQKQANLVLKIIEQLPSAKESPDKFLEVCTWVDQ | | |
| | | | IAALNDSKTRKTTSETVRAVLVSLSEKKRSSP | | |
| SCOH-ro31- | 7E33S38Y | 8K19S28E | MANTKYNEEFLLYLAGFVDGDGSIIAQIKPNQSSKFKHYLSLTFRV | 118 | Nd |
| b56-D | 44R68Y70 | 38R40K43 | TQKTQRRWFLDKLVDEIGVGYVYDSGSVSDYNLSEIKPLRNLLTQL | | |
| (pCLS6299) | S77N85R | L44K54L | QPFLELKQKQANLVLKIIQLPSAKESPDKFLEVCTWVDQIAALNDS | | |
| | 87L96E | 61R70E75 | KTRKTTSETVRAVLDSLSEKKKPSPAAGGSDKYNQALSKYNQALSK | | |
| | 161P | N81V96R | YNQALSGGGGSNKKFLLYLAGFVDSDGSIIAQIEPNQSYKFKHRLK | | |
| | | 132V153 | LTLKVTQKTQRRWLLDKLVDRIGVGYVRDEGSVSNYILSEVKPLHN | | |
| | | V160R | FLTQLQPFLRLKQKQANLVLKIIEQLPSAKESPDKFLEVCTWVDQV | | |
| | | | AALDNSKTRKTTSETVRAVLVSLSEKKRSSP | | |
| SCOH-ro31- | 7E33S38Y | 8K19S28E | MANTKYNEEFLLYLAGFVDGDGSIIAQIKPNQSSKFKHYLSLTFRV | 119 | + |
| b1-A | 44R66H68 | 38R40K43 | TQKTQRRWFLDKLVDEIGVGHVYDSGSVSDYNLSEIKPLHNLLTQL | | |
| (pCLS6300) | Y70S77N | L44K54L | QPLLELKQKQANLVLKIIEQLPSAKESPDKFLEVCTWVDQIAALND | | |
| | 87L94L96 | 61R70E75 | SKTRKTTSETVRAVLDSLSGKKKSSPAAGGSDKYNQALSKYNQALS | | |
| | E157G | N81V96R | KYNQALSGGGGSNKKFLLYLAGFVDSDGSIIAQIEPNQSYKFKHRL | | |
| | | 153V160G | KLTLKVTQKTQRRWLLDKLVDRIGVGYVRDEGSVSNYILSEVKPLH | | |
| | | | NFLTQLQPFLRLKQKQANLVLKIIEQLPSAKESPDKFLEVCTWVDQ | | |
| | | | IAALNDSKTRKTTSETVRAVLVSLSEKKGSSP | | |
| SCOH-ro31- | 7E33S38Y | 8K19S28E | MANTKYNEEFLLYLAGFVDGDGSIIAQIKPNQSSKFKHYLSLTFRV | 120 | + |
| b1-B | 44R66H68 | 38R40K43 | TQKTQRRWFLDKLVDEIGVGHVYDSGSVSDYNLSEIKPLHNLLTQL | | |
| (pCLS6301) | Y70S77N | L44K54L | QPLLELKQKQANLVLKIIEQLPSAKESPDKFLEVCTWVDQVAALND | | |
| | 87L94L96 | 61R70E75 | SKTRKTTSETVRAVLDSLSGKKKSSPAAGGSDKYNQALDKYNQALS | | |
| | E132V157 | N81V96R | KYNQALSGGGGSNKKFLLYLAGFVDSDGSIIAQIEPNQSYKFKHRL | | |
| | G | 153V160G | KLTLKVTQKTQRRWLLDKLVDRIGVGYVRDEGSVSNYILSEVKPLH | | |
| | | | NFLTQLQPFLRLKQKQANLVLKIIEQLPSAKESPDKFLEVCTWVDQ | | |
| | | | IAALNDSKTRKTTSETVRAVLVSLSEKKGSSP | | |
| pCLS6302- | 7E33S38Y | 8K19S28E | MANTKYNEEFLLYLAGFVDGDGSIIAQIKPNQSSKFKHYLSLTFRV | 121 | + |
| SCOH-ro31- | 44R66H68 | 38R40K43 | TQKTQRRWFLDKLVDEIGVGHVYDSGSVSDYNLSEIKPLHNLLTQL | | |
| b1-D | Y70S77N | L44K54L | QPLLELKQKQANLVLKIIEQLPSAKESPDKFLEVCTWVDQIAALND | | |
| (pCLS6302) | 87L94L96 | 61R70E75 | SKTRKTTSETVRAVLDSLSGKKKSSPAAGGSDKYNQALSKYNQALS | | |
| | E157G | N81V96R | KYNQALSGGGGSNKKFLLYLAGFVDSDGSIIAQIEPNQSYKFKHRL | | |
| | | 132V153 | KLTLKVTQKTQRRWLLDKLVDRIGVGYVRDEGSVSNYILSEVKPLH | | |
| | | V160G | NFLTQLQPFLRLKQKQANLVLKIIEQLPSAKESPDKFLEVCTWVDQ | | |
| | | | VAALNDSKTRKTTSETVRAVLVSLSEKKGSSP | | |
| SCOH-ro31- | 7E33S38Y | 8K19S28E | MANTKYNEEFLLYLAGFVDGDGSIIAQIKPNQSSKFKHYLSLTFRV | 122 | Nd |
| b56-B | 44R68Y70 | 38R40K43 | TQKTQRRWFLDKLVDEIGVGYVYDSGSVSDYNLSEIKPLRNLLTQL | | |
| (pCLS6304) | S77N85R | L44K54L | QPFLELKQKQANLVLKIIEQLPSAKESPDKFLEVCTWVDQVAALND | | |
| | 87L96E | 61R70E75 | SKTRKTTSETVRAVLDSLSEKKKPSPAAGGSDKYNQALSKYNQALS | | |
| | 132V161P | N81V96R | KYNQALSGGGGSNKKFLLYLAGFVDSDGSIIAQIEPNQSYKFKHRL | | |
| | | 153V160R | KLTLKVTQKTQRRWLLDKLVDRIGVGYVRDEGSVSNYILSEVKPLH | | |
| | | | NFLTQLQPFLRLKQKQANLVLKIIEQLPSAKESPDKFLEVCTWVDQ | | |
| | | | IAALNDSKTRKTTSETVRAVLVSLSEKKRSSP | | |
| SCOH-ro31- | 7E33S38Y | 8K19S28E | MANTKYNEEFLLYLAGFVDGDGSIIAQIKPNQSSKFKHYLSLTFRV | 123 | Nd |
| b1-E | 44R66H68 | 38R40K43 | TQKTQRRWFLDKLVDEIGVGYVYDSGSVSDYNLSEIKPLRNLLTQL | | |
| (pCLS6316) | Y70S77N | L44K54L | QPFLELKQKQANLVLKIIEQLPSAKESPDKFLEVCTWVDQVAALND | | |
| | 80K87L94 | 61R70E75 | SKTRKTTSETVRAVLDSLSEKKKPSPAAGGSDKYNQALSKYNQALS | | |
| | L96E132 | N81V96R | KYNQALSGGGGSNKKFLLYLAGFVDSDGSIIAQIEPNQSYKFKHRL | | |
| | V157G | 105A132V | KLTLKVTQKTQRRWLLDKLVDRIGVGYVRDEGSVSNYILSEVKPLH | | |
| | | 153V160G | NFLTQLQPFLRLKQKQANLVLKIIEQLPSAKESPDKFLEVCTWVDQ | | |
| | | | IAALNDSKTRKTTSETVRAVLVSLSEKKRSSP | | |

Any of the single chain molecules active in Yeast on rho31.1 target can be considered for genome engineering at Rho31 locus including insertion of transgenes, RHO promoter engineering, gene modification and gene correction.

### LIST OF REFERENCES CITED IN THE DESCRIPTION

1. Sullivan et al. Invest Ophthalmol V is Sci 2006 47(7): 3052-3064
2. Hims et al. Dev Ophthalmol 2003 37:109-25
3. Bunker et al. Am J Ophthalmol 1984 97:357-65
4. Ayuso et al. Clin Genet. 1995 48:120-2
5. Van Soest et al. Surv Ophthalmol 1999 43:321-34
6. Audo et al.
7. O'Reilly et al. Am J Hum Genet. 2007 81(1): 127-35
8. Palfi et al. Hum Gene Ther 2010 21(3): 311-23
9. Takahashi et al. Methods Mol Biol 2004 246: 439-49
10. Capecchi et al. Trends Genet. 1989 5(3): 70-6.
11. Smithies et al. Nat Med 2001 7(10): 1083-6
12. De Semir D et al. J Gene Med 2003 5: 625-639
13. Goncz et al. Gene Therapy 2001 8: 961-965
14. Sangiuolo et al. BMC Med Genet. 2002 3-8
15. Bruscia et al. Gene Ther 2002 9: 683-685
16. De Semir and Aran Oligonucleotides 2003 13: 261-269
17. Thierry and Dujon Nucleic Acids Res 1992 20: 5625-5631
18. Puchta et al. Nucleic Acids Res 1993 21: 5034-5040
19. Rouet et al. Mol Cell Biol 1994 14: 8096-8106
20. Choulika et al. Mol Cell Biol 1995 15: 1968-1973
21. Puchta et al. Proc Natl Acad Sci U.S.A 1996 93: 5055-5060
22. Sargent et al. Mol Cell Biol 1997 17: 267-277
23. Cohen-Tannoudji et al. Mol Cell Biol 1998 18: 1444-1448
24. Donoho et al. Mol Cell Biol 1998 18: 4070-4078
25. Elliott et al. Mol Cell Biol 1998 18: 93-101
26. Chevalier and Stoddard Nucleic Acids Res 2001 29: 3757-3774
27. Smith et al. Nucleic Acids Res 1999 27: 674-681
28. Bibikova et al. Mol Cell Biol 2001 21: 289-297
29. Bibikova et al. Genetics 2002 161: 1169-1175
30. Bibikova et al. Science 2003 300: 764
31. Porteus and Baltimore Science 2003 300: 763
32. Alwin et al. Mol Ther 2005 12: 610-617
33. Urnov et al. Nature 2005 435: 646-651
34. Porteus M. H. Mol Ther 2006 13: 438-446
35. Pabo et al. Annu Rev Biochem 2001 70: 313-340
36. Jamieson et al. Nat Rev Drug Discov 2003 2: 361-368
37. Rebar and Pabo Science 1994 263: 671-673
38. Kim and Pabo Proc Natl Acad Sci USA 1998 95: 2812-2817
39. Klug et al. Proc Natl Acad Sci USA 1994 91: 11163-11167
40. Isalan and Klug Nat Biotechnol 2001 19: 656-660
41. Catto et al. Nucleic Acids Res 2006 34: 1711-1720
42. Chevalier et al. Nat Struct Biol 2001 8: 312-316
43. Chevalier et al. J Mol Biol 2003 329: 253-269
44. Moure et al. J Mol Biol 2003 334: 685-693,
45. Silva et al. J Mol Biol 1999 286: 1123-1136
46. Bolduc et al. Genes Dev 2003 17: 2875-2888
47. Ichiyanagi et al. J Mol Biol 2000 300: 889-901
48. Moure et al. Nat Struct Biol 2002 9: 764-770
49. Chevalier et al. Mol Cell 2002 10: 895-905
50. Epinat et al. Nucleic Acids Res 2003 31: 2952-62
51. Seligman et al. Genetics 1997 147: 1653-1664
52. Sussman et al. J Mol Biol 2004 342: 31-41
53. Arnould et al. J Mol Biol 2006 355: 443-458
54. Rosen et al. Nucleic Acids Res 2006 34: 4791-4800
55. Smith et al. Nucleic Acids Res 2006 34 e149
56. Doyon et al. J Am Chem Soc 2006 128: 2477-2484
57. Gimble et al. J Mol Biol 2003 334: 993-1008
58. Ashworth et al. Nature 2006 441: 656-659
59. Argast et al. J Mol Biol 1998 280: 345-353
60. Jurica et al. Mol Cell 1998 2: 469-476
61. Chevalier et al. Biochemistry 2004 43: 14015-14026
62. Chames et al. Nucleic Acids Res 2005 33 e178
63. Arnould et al. J Mol Biol 2007 371 49-65
64. Chevalier et al. J Mol Biol 2003 329: 253-269
65. Prieto et al. Nucleic Acids Res Epub 22 Apr. 2007
66. Fallaux et al. Hum Gene Ther 1998 9: 1909-1917
67. Akagi et al. Nucleic Acids Res 1997 25 (9): 1766-73
68. Zhu X D et al. J Biol Chem 1995 270
69. Zeitz et al. Invest Ophthalmol V is Sci 2008 49(9): 4105-14 Epub 2008 May 16
70. Bonetta The Scientist 2002 16:38
71. Ford et al. Gene Ther 2001 8: 1-4
72. Wadia and Dowdy Curr Opin Biotechnol 2002 13: 52-56
73. Gimenez et al. Pigment Cell Res 2004 17(4): 363-370
74. Steuer et al. Chembiochem 2004 5: 206-13

1. An I-CreI variant, comprising at least two I-CreI monomers wherein at least one of the two I-CreI monomers comprises at least two substitutions, one in each of two functional subdomains of a LAGLIDADG core domain situated from positions 26 to 40 and 44 to 77 of I-CreI, the variant being able to cleave a DNA target sequence selected from the group consisting of the sequences SEQ ID NO: 8 to 13, 20 to 25, 32 to 37, and 86 to 91 from a Rhodopsin gene (RHO), and wherein the I-CreI variant is obtained by a method comprising:
(a) constructing a first series of I-CreI variants comprising a substitution of at least one position selected from the group consisting of 26, 28, 30, 32, 33, 38 and 40 of a first functional subdomain of the LAGLIDADG core domain situated from positions 26 to 40 of I-CreI,
(b) constructing a second series of I-CreI variants comprising a substitution of at least one position selected from the group consisting of 44, 68, 70, 75 and 77 of a second functional subdomain of the LAGLIDADG core domain situated from positions 44 to 77 of I-CreI,
(c) selecting, screening, or selecting and screening the variants from the first series of (a) which are able to cleave a mutant I-CreI site wherein
(i) a nucleotide triplet in positions −10 to −8 of the I-CreI site has been replaced with a nucleotide triplet which is present in positions −10 to −8 of the DNA target sequence from RHO and
(ii) a nucleotide triplet in positions +8 to +10 has been replaced with a reverse complementary sequence of a nucleotide triplet which is present in position −10 to −8 of the DNA target sequence from RHO,
(d) selecting, screening, or selecting and screening the variants from the second series of (b) which are able to cleave a mutant I-CreI site wherein
(i) a nucleotide triplet in positions −5 to −3 of the I-CreI site has been replaced with a nucleotide triplet which is present in positions −5 to −3 of the DNA target sequence from RHO and
(ii) a nucleotide triplet in positions +3 to +5 has been replaced with a reverse complementary sequence of the nucleotide triplet which is present in position −5 to −3 of the DNA target sequence from RHO,
(e) selecting, screening, or selecting and screening the variants from the first series of (a) which are able to cleave a mutant I-CreI site wherein
(i) a nucleotide triplet in positions +8 to +10 of the I-CreI site has been replaced with a nucleotide triplet which is present in positions +8 to +10 of the DNA target sequence from RHO and
(ii) a nucleotide triplet in positions −10 to −8 has been replaced with a reverse complementary sequence of the nucleotide triplet which is present in position +8 to +10 of the DNA target sequence from RHO,
(f) selecting, screening, or selecting and screening the variants from the second series of (b) which are able to cleave a mutant I-CreI site wherein
(i) a nucleotide triplet in positions +3 to +5 of the I-CreI site has been replaced with a nucleotide triplet which is present in positions +3 to +5 of the DNA target sequence from RHO and
(ii) a nucleotide triplet in positions −5 to −3 has been replaced with a reverse complementary sequence of the nucleotide triplet which is present in position +3 to +5 of the DNA target sequence from RHO, and
wherein the method further comprises (g), (h), or (g) and (h) comprising:
(g) combining in a single variant, the mutation or mutations in positions 26 to 40 and 44 to 77 of two variants from (c) and (d), to obtain a novel homodimeric I-CreI variant which cleaves a sequence wherein
(i) the nucleotide triplet in positions −10 to −8 is identical to the nucleotide triplet which is present in positions −10 to −8 of the DNA target sequence from RHO,
(ii) the nucleotide triplet in positions +8 to +10 is identical to the reverse complementary sequence of the nucleotide triplet which is present in positions −10 to −8 of the DNA target sequence from RHO,
(iii) the nucleotide triplet in positions −5 to −3 is identical to the nucleotide triplet which is present in positions −5 to −3 of the DNA target sequence from RHO and
(iv) the nucleotide triplet in positions +3 to +5 is identical to the reverse complementary sequence of the nucleotide triplet which is present in positions −5 to −3 of the DNA target sequence from RHO, and
(h) combining in a single variant, the mutation or mutations in positions 26 to 40 and 44 to 77 of two variants from (e) and (f), to obtain a novel homodimeric I-CreI variant which cleaves a sequence wherein
(i) the nucleotide triplet in positions +8 to +10 of the I-CreI site has been replaced with the nucleotide triplet which is present in positions +8 to +10 of the DNA target sequence from RHO,
(ii) the nucleotide triplet in positions −10 to −8 is identical to the reverse complementary sequence of the nucleotide triplet in positions +8 to +10 of the DNA target sequence from RHO,
(iii) the nucleotide triplet in positions +3 to +5 is identical to the nucleotide triplet which is present in positions +3 to +5 of the DNA target sequence from RHO,
(iv) the nucleotide triplet in positions −5 to −3 is identical to the reverse complementary sequence of the nucleotide triplet which is present in positions +3 to +5 of the DNA target sequence from RHO, and
wherein the method further comprises:
(i) combining at least one variant obtained in (g) or (h) to form a heterodimer, and
(j) selecting, screening, or selecting and screening the heterodimer from (i) which is able to cleave the DNA target sequence from RHO.

2. (canceled)

3. (canceled)

4. (canceled)

5. (canceled)

6. (canceled)

7. The variant of claim 1, which comprises a substitution in positions 137 to 143 of I-CreI that modifies the specificity of the variant towards the nucleotide in at least one position selected from the group consisting of positions ±1 to 2, ±6 to 7 and ±11 to 12 of the target site in RHO.

8. The variant of claim 1, which comprises a substitution on the entire I-CreI sequence that improves binding, cleavage, or binding and cleavage properties of the variant towards the DNA target sequence from RHO.

9. The variant of claim 1, wherein the substitutions replacements of the initial amino acids wherein the amino acids are selected from the group consisting of A, D, E, F, G, H, I, K, M, N, P, Q, R, S, T, Y, C, W, L and V.

10. The variant of claim 1, wherein the variant is a heterodimer, resulting from the association of a first and a second monomer comprising different mutations in positions 26 to 40 and 44 to 77 of I-CreI, wherein the heterodimer is able to cleave a non-palindromic DNA target sequence from RHO.

11. The variant of claim 10, wherein the variant is an obligate heterodimer, wherein the first and the second monomer, respectively, further comprises a D137R mutation and a R51D mutation.

12. The variant of claim 10, wherein the variant is an obligate heterodimer, wherein the first monomer further comprises K7R, E8R, E61R, K96R and L97F or K7R, E8R, F54W, E61R, K96R and L97F mutations and the second monomer further comprises the K7E, F54G, L58M and K96E or K7E, F54G, K57M and K96E mutations.

13. The variant according to claim 1, wherein the variant comprises a single polypeptide chain comprising two monomers or core domains of one or two variants.

14. The variant of claim 13, wherein the variant comprises the first and the second monomers connected by a peptide linker.

15. The variant of claim 1, wherein the DNA target is selected from the group consisting of the SEQ ID NO: 8 to 13, 20 to 25, 32 to 37, 86 to 91.

16. The variant of claim 1, wherein at least one of the I-CreI monomers are selected from the group consisting of SEQ ID NO: 40 to 65, SEQ ID NO: 92 to 103 and SEQ ID NO: 105 to 116.

17. The variant according to claim 14, wherein the variant is selected from the group consisting of SEQ ID NO: 66 to 76, SEQ ID NO: 104 and SEQ ID NO: 117 to 123.

18. A polynucleotide fragment encoding the variant of claim 1.

19. An expression vector comprising a polynucleotide fragment of claim 18.

20. The vector of claim 19, comprising a sequence to be introduced flanked by sequences sharing homologies with the regions surrounding the DNA target sequence from RHO.

21. The vector of claim 20, wherein the sequence to be introduced is a sequence which inactivates RHO.

22. The vector of claim 21, wherein the sequence which inactivates RHO comprises in the 5′ to 3′ orientation:
a first transcription termination sequence and a marker cassette comprising a promoter,
a marker open reading frame and a second transcription termination sequence, and
the sequence interrupts the transcription of a coding sequence.

23. The vector of claim 19, wherein the sequence sharing homologies with the regions surrounding DNA target sequence from RHO is a fragment of RHO comprising sequences upstream and downstream of a cleavage site, so as to allow the deletion of coding sequences flanking the cleavage site.

24. A host cell which comprises the polynucleotide of claim 18.

25. A host cell which comprises the vector of claim 19.

26. A non-human transgenic animal which comprises the polynucleotide of claim 18.

27. A non-human transgenic animal which comprises the vector of claim 19.

28. A transgenic plant which comprises the polynucleotide of claim 18.

29. A transgenic plant which comprises the vector of claim 19.

30. A method of treatment of a genetic disease caused by a mutation in RHO comprising administering to a subject in need thereof an effective amount of the variant of claim 1.


SEQUENCE LISTING (123 sequences)

SEQ ID NO: 1 - protein, 163 aa, Chlamydomonas reinhardtii
MNTKYNKEFLLYLAGFVDGDGSIIAQIKPNQSYKFKHQLSLTFQVTQKTQRRWFLDKLVDEIGVGYVRDRGSVSDYILSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 2 - DNA, 22 bp, Artificial
  C1221 target
caaaacgtcgtacgacgttttg

SEQ ID NO: 3 - protein, 167 aa, Artificial
  I-CreI  N75
MANTKYNKEFLLYLAGFVDGDGSIIAQIKPNQSYKFKHQLSLAFQVTQKTQRRWFLDKLVDEIGVGYVRDRGSVSDYILSEIKPLHNFLTQLQPFLKLKQKQANLVLKIIWRLPSAKESPDKFLEVCTWVDQIAALNDSKTRKTTSETVRAVLDSLSEKKKSSPAAD

SEQ ID NO: 4 - DNA, 22 bp, Artificial
  10TTC_P target
cttcacgtcgtacgacgtgaag

SEQ ID NO: 5 - DNA, 22 bp, Artificial
  10GTG_P target
cgtgacgtcgtacgacgtcacg

SEQ ID NO: 6 - DNA, 22 bp, Artificial
  5CAC_P target
caaaaccacgtacgtggttttg

SEQ ID NO: 7 - DNA, 22 bp, Artificial
  5GTA_P target
caaaacgtagtactacgttttg

SEQ ID NO: 8 - DNA, 22 bp, Artificial
  Rho34.1 target
cttcctcacgctctacgtcacc

SEQ ID NO: 9 - DNA, 22 bp, Artificial
  Rho34.2 target
cttcctcacgtactacgtcacc

SEQ ID NO: 10 - DNA, 22 bp, Artificial
  Rho34.3 target
cttcctcacgtacgtgaggaag

SEQ ID NO: 11 - DNA, 22 bp, Artificial
  Rho34.5 target
cttcctcacgctcgtgaggaag

SEQ ID NO: 12 - DNA, 22 bp, Artificial
  Rho34.4 target
ggtgacgtagtactacgtcacc

SEQ ID NO: 13 - DNA, 22 bp, Artificial
  Rho34.6 target
ggtgacgtagctctacgtcacc

SEQ ID NO: 14 - DNA, 22 bp, Artificial
  10TTC-5CAC_P target
cttcaccacgtacgtggtgaag

SEQ ID NO: 15 - DNA, 22 bp, Artificial
  10GTG-5GTA_P target
cgtgacgtagtactacgtcacg

SEQ ID NO: 16 - DNA, 22 bp, Artificial
  10CAG_P target
ccagacgtcgtacgacgtctgg

SEQ ID NO: 17 - DNA, 22 bp, Artificial
  10TGC_P target
ctgcacgtcgtacgacgtgcag

SEQ ID NO: 18 - DNA, 22 bp, Artificial
  5ACC_P target
caaaacaccgtacggtgttttg

SEQ ID NO: 19 - DNA, 22 bp, Artificial
  5TCT_P target
caaaactctgtacagagttttg

SEQ ID NO: 20 - DNA, 22 bp, Artificial
  Rho_7.1 target
tcagccaccacacagaaggcag

SEQ ID NO: 21 - DNA, 22 bp, Artificial
  Rho_7.2 target
tcagccaccgtacagaaggcag

SEQ ID NO: 22 - DNA, 22 bp, Artificial
  Rho_7.3 target
tcagccaccgtacggtggctga

SEQ ID NO: 23 - DNA, 22 bp, Artificial
  Rho_7.4 target
ctgccttctgtacagaaggcag

SEQ ID NO: 24 - DNA, 22 bp, Artificial
  Rho_7.5 target
tcagccaccacacggtggctga

SEQ ID NO: 25 - DNA, 22 bp, Artificial
  Rho_7.6 target
ctgccttctacacagaaggcag

SEQ ID NO: 26 - DNA, 22 bp, Artificial
  10CAG-5ACC_P target
ccagacaccgtacggtgtctgg

SEQ ID NO: 27 - DNA, 22 bp, Artificial
  10TGC-5TCT_P target
ctgcactctgtacagagtgcag

SEQ ID NO: 28 - DNA, 22 bp, Artificial
  10GAT_P target
cgatacgtcgtacgacgtatcg

SEQ ID NO: 29 - DNA, 22 bp, Artificial
  5CAC_P target
caaaaccacgtacgtggttttg

SEQ ID NO: 30 - DNA, 22 bp, Artificial
  10CCT_P target
ccctacgtcgtacgacgtaggg

SEQ ID NO: 31 - DNA, 22 bp, Artificial
  5CTG_P target
caaaacctggtaccaggttttg

SEQ ID NO: 32 - DNA, 22 bp, Artificial
  Rho_36.1 target
agatcccacttaacagagagga

SEQ ID NO: 33 - DNA, 22 bp, Artificial
  Rho_36.2 target
agatcccacgtaccagagagga

SEQ ID NO: 34 - DNA, 22 bp, Artificial
  Rho_36.3 target
agatcccacgtacgtgggatct

SEQ ID NO: 35 - DNA, 22 bp, Artificial
  Rho_36.4 target
tcctctctggtaccagagagga

SEQ ID NO: 36 - DNA, 22 bp, Artificial
  Rho_36.5 target
agatcccacttaagtgggatct

SEQ ID NO: 37 - DNA, 22 bp, Artificial
  Rho_36.6 target
tcctctctgttaacagagagga

SEQ ID NO: 38 - DNA, 22 bp, Artificial
  10GAT-5CAC_P target
cgataccacgtacgtggtatcg

SEQ ID NO: 39 - DNA, 22 bp, Artificial
  10CCT-5CTG_P target
ccctacctggtaccaggtaggg

SEQ ID NO: 40 - protein, 167 aa, artificial sequence
  Rho34.5 I-CreI variant
MANTKYNKEFLLYLAGFVDGDGSIIAQIKPNQTCKFKHHLSLTFVVTQKTQRRWFLDKLVDEIGVGYVYDSGSVSRYVLSEIKPLHNFLTQLQPFLKLKQRQANLVLKIIEQLPSAKESPDKFLEVCTWVDQIAALNDSKTRKTTSETVRAVLDSLSEKKKSSPAAD

SEQ ID NO: 41 - protein, 167 aa, artificial sequence
  Rho34.5 I-CreI variant
MANTKYNKEFLLYLAGFVDGDGSIIAQIKPNQTCKFKHHLSLTFVVTQKTQRRWFLDKLVDEIGVGYVYDSGSVSRYVLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 42 - protein, 167 aa, artificial sequence
  Rho34.5 I-CreI variant
MANTKYNKEFLLYLAGFVDGDGSIIAQIKPNRTCKFKHHLSLTFVVTQKTQRRWFLDKLVDEIGVGYVYDSGSVSRYVLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 43 - protein, 167 aa, artificial sequence
  Rho34.5 I-CreI variant
MVNTKYNKEFLLYLAGFVDGDGSIIAQIKPNQTCKFKHHLSLTFVVTQKTQRRWFLDKLVDEIGVGYVYDSGSVSRYVLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 44 - protein, 167 aa, artificial sequence
  Rho34.5 I-CreI variant
MANTKYNKEFLLYLAGFVDGDGSIIAQIKPNQTCKFKHHLSLTFVVTQKTQRRWSLDKLVDEIGVGYVYDSGSVSRYVLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 45 - protein, 167 aa, artificial sequence
  Rho34.5 I-CreI variant
MANTKYNKEFLLYLAGFVDGDGSIIAQIKPNQTCKFKHHLSSTFVVTQKTQRRWFLDKLVDEIGVGYVYDSGSVSRYVLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 46 - protein, 167 aa, artificial sequence
  Rho34.5 I-CreI variant
MANTKYNKEFLLYLAGFVDGDGSVIAQIKPNQTCKFKHHLSLTFVVTQKTQRRWFLDKLVDEIGVGYVYDSGPVSRYVLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 47 - protein, 167 aa, artificial sequence
  Rho34.5 I-CreI variant
MANTKYNKEFLLYLAGFVDGDGSIIAQIKPNQTCKFKHHLSLTFVVTQKTQRRWFLDKLVDEIGVGYVYDSGSVSRYVLSEIKPLHNFLTQLQPFLKLKQKQANLVLKIIEQLPSAKESPDKFLEVCTWVDQIAALNDSKTRKTTSETVRAVLGSLSEKKKSSPAAD

SEQ ID NO: 48 - protein, 167 aa, artificial sequence
  Rho34.6 I-CreI variant
MANTKYDKEFLLYLAGFVDGDGSIIAQIKPNQHHKFKHALSLTFSVGQKTQRRWFLDKLVDEIGVGYVRDSGSMSEYYLSEIKPLHNFLTQLQPFLKLKQKQANLVLKIIEQLPSAKGSPDKFLEVCTWVDQVAALNDSKTRKTTSETVRAVLDSLSEKKKSSPAAD

SEQ ID NO: 49 - protein, 167 aa, artificial sequence
  Rho34.6 I-CreI variant
MANTKYNKEFLLYLAGFVDGDGSIIAQIKPNQHHKFKHALSLTFSVGQKTQRRWFLDKLVDEIGVGHVRDSGSMSEYYLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 50 - protein, 167 aa, artificial sequence
  Rho34.6 I-CreI variant
MANTKYNKEFLLYLAGFVDGDGSIIAQIKPNQHHKFKHALSLTFSVGQKTQRRWFLDKLADEIGVGYVRDSGSMSEYCLSGIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 51 - protein, 167 aa, artificial sequence
  Rho34.6 I-CreI variant
MANTKYNKEFLLYLAGFVDGDGSIIAQIKPNQHHKFKHALSLTFSVGQKTQRRWFLDKLVDEIGVGHVRDSGSMSEYYLSEIKPLHNFLTQLQPFLKLKQKQANLALKIIEQLPSAKESPDKFLEVCTWVDQIAALNDSKTRKTTSETVRAVLDSLSEKKKSSPAAD

SEQ ID NO: 52 - protein, 167 aa, artificial sequence
  Rho34.6 I-CreI variant
MANTKYNKEFLLYLAGFVDGDGSIIAQIKPNQHHKFKHALSLTFSVGQKTQRRWFLDKLVDEIGVGHVRNSGSMSEYYLSEIKPLHNFLTQLQPFLKLKQKQANLVLKIIGQLPSAKESPDKFLEVCTWVDQIAALNDSKTRKTTSETVRAVLDSLSEKKKSSPAAD

SEQ ID NO: 53 - protein, 167 aa, artificial sequence
  Rho_7.6 I-CreI variant
MANTKYNKEFLLYLAGFADGDGSIIAQISPNQSSKFKHRLRLTFQVTQKTQRRWLLDKLVDEIGVGYVSDSGSVSNYRLSEIEPLHNFLTQLQPFLKLKQKQANLVLKIIEQLPSAKESPDKFLEVCTWVDQIAALNDSKTRKTTSETVRAALDSLSEKKKSSPAAD

SEQ ID NO: 54 - protein, 167 aa, artificial sequence
  Rho_7.6 I-CreI variant
MANTKYNKEFLLYLAGFVDGDGSIIAQISPNQSSKFKHRLRLTFQVTQKTQRRWLLDKLVDEIGVGYVSDSGSVSNYRLSEIEPLHNLLTQLQPFLKLKQKQANLVLKIIEQLPSAKESPDKFLEACTWVDRIAALNDSKTRKTTSETVRAVLDSLSEKKKSSPAAD

SEQ ID NO: 55 - protein, 167 aa, artificial sequence
  Rho_7.6 I-CreI variant
MANTKYNKEFLLYLAGFVDGDGSIIAQISPNQSSKFKHRLRLTFQVTQKTQRRWLLDKLVDEIGVGYVSDSGSVSNYRLSEIEPLHNFLTQLQPFLKLKQKQANLVLKIIEQLPSAKESPDKFLEVCTWVDQIAALNDSKTRRTTSETVRAALDSLSEKKKSSPAAD

SEQ ID NO: 56 - protein, 167 aa, artificial sequence
  Rho_7.6 I-CreI variant
MANTKYNKEFLLYLAGFVDGDGSIIAQISPNQSSKFKHRLRLTFQVTQKTQRRWLLDKLVDEIGVGYVSDSGSVSNYRLSEIEPLHNFLAQLQPFLKLKQKQANLVLKIIEQLPSAKESPDKFLEVCTWVDRIAALNDSKTRKTTSETVRAVLDSLSEKKKSSPAAD

SEQ ID NO: 57 - protein, 167 aa, artificial sequence
  Rho_7.6 I-CreI variant
MANTKYNKEFLLYLAGFVDGDGSIIAQISPNQSSKFKHRLRLTFQVTQKTQRRWLLDKLLDEIGVGYVSDSGSVSNYRLSEIEPLHNFLTQLQPFLKLKQKQANLVLKIIEQLPSAKESPDKFLEVCTWVDRIAALNDSKTRKTTSETVRAVLDSLSEKKKSSPAAD

SEQ ID NO: 58 - protein, 167 aa, artificial sequence
  Rho_7.6 I-CreI variant
MANTKYNKEFLLYLAGFVDGDGSIIAQISPNQSSKFKHRLRLTFQVTQKTQRRWLLDKLVDEIGVGYVSDSGSVSNYRLSEIEPLHNFLTQLQPFLKLKQKQANLVLKIIEQLPSAKESPDKFLEVCTWVDRIAALNDSKTRKTTSETVRAVLDSLSEKKKSSPAAD

SEQ ID NO: 59 - protein, 167 aa, artificial sequence
  Rho_7.6 I-CreI variant
MANTKYNKEFLLYLAGFVDGDGSIIAQISPNQSSKFKHRLRLTFQVTQKTQRRWLLDKLVDEIGVGYVSDSGSVSNYRLSEIEPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 60 - protein, 167 aa, artificial sequence
  Rho_7.6 I-CreI variant
MANTKYNKEFLLYLAGFVDGDGSIIAQISPNQSSKFKHRLRLTFQVTQKTQRRWLLDKLVDEIGVGYVSDSGSVSNYRLSEIEPLHNFLTQLQPFLKLKQKQANLVLKIIEQLPSAKESPDKFLEVCTWVDQIAALNDSKTRKTTSETVRAALDSLSEKKKSSPTAD

SEQ ID NO: 61 - protein, 167 aa, artificial sequence
  Rho_7.6 I-CreI variant
MANTKYNKEFLLYLAGFVDGDGSIIAQISPNQSSKFKHRLRLTFQVTQKTQRRWLLDKLVDEIGVGYVSDSGSVSNYRLSEIEPLHNFLTQLQPFLKLKQKQANLVLKIIEQLPSAKESPDKFLEVCTWVDQIAALNDSKTRKTTSETVRAALDSLSEKKKSSPAAD

SEQ ID NO: 62 - protein, 167 aa, artificial sequence
  Rho_7.5 I-CreI variant
MTNTKYNKELLLYLAGFVDGDGSIIAQIKPNQSNKFKHYLRLTLKVTQKTQRRWLLDELVDEIGVGYVYDSGSVSYYQLSEIKPLRSFLAQLQPFLKLKQKQANLVLKIIEQLPSAKESPDKFLEVCTWVDQIAALNDSKTRKTTSETVRAVLDTLSEKKKSSPAAD

SEQ ID NO: 63 - protein, 167 aa, artificial sequence
  Rho_7.5 I-CreI variant
MANTKYNKELLLYLAGFVDGDGSIIAQIKPNQSNKFKHYLRLTLKVTQKTQRRWLLDELVDEIGVGYVYDSGSVSYYQLSEIKPLRSFLAQLQPFLKLKQKQANLVLKIIEQLPSAKESPDKFLEVCTWVDQIAALNDSKTRKTTSETVRAVLDSLSEEKKSSPAAD

SEQ ID NO: 64 - protein, 167 aa, artificial sequence
  Rho_7.5 I-CreI variant
MANTKYNKELLLYLAGFVDGDGSIVAQIKPNQSNKFKHYLRLTLKVTQKTQRRWLLDELVDEIGVGYVYDSGSVSYYQLSEIKPLRSFLAQLQPFLKLKQKQANLVLKIIEQLPSAKESPDKFLEVCTWVDQIAALNDSKTRKTTSETVRAVLDSLGEKKKSSPAAD

SEQ ID NO: 65 - protein, 167 aa, artificial sequence
  Rho_7.5 I-CreI variant
MANTKYNKELLLYLAGFVDGDGSIIAQIKPNQSSKFKHYLRLTLKVTQKTQRRWLLDELVDEIGVGYVYDSGSVSYYQLSEIKPLHSFLAQLQPFLKLKQKQANLVLKIIEQLPSAKESPDKFLEVCTWVDQIAALNDSKTRKTTSETVHAVLDSLSEKKKSSPAAD

SEQ ID NO: 66 - protein, 354 aa, artificial sequence
  SCOH-ro34-b56-D
MANTKYNEEFLLYLAGFVDGDGSIIAQIKPNQTCKFKHHLSSTFVVTQKTQRRWFLDKLVDEIGVGYVYDSGSVSRYVLSEIKPLHNFLTQLQPFLELKQKQANLVLKIIEQLPSAKESPDKFLEVCTWVDQIAALNDSKTRKTTSETVRAVLDSLSEKKKSSPAAGGSDKYNQALSKYNQALSKYNQALSGGGGSNEEFLLYLAGFVDGDGSIIAQIKPNQTCKFKHHLSSTFVVTQKTQRRWFLDKLVDEIGVGYVYDSGSVSRYVLSEIKPLHNFLTQLQPFLELKQKQANLVLKIIEQLPSAKESPDKFLEVCTWVDQIAALNDSKTRKTTSETVRAVLDSLSEKKKSSP

SEQ ID NO: 67 - protein, 354 aa, artificial sequence
  SCOH-ro34-b56-A
MANTKYNEEFLLYLAGFVDGDGSIIAQIKPNQTCKFKHHLSSTFVVTQKTQRRWFLDKLVDEIGVGYVYDSGSVSRYVLSEIKPLHNFLTQLQPFLELKQKQANLVLKIIEQLPSAKESPDKFLEVCTWVDQIAALNDSKTRKTTSETVRAVLDSLSEKKKSSPAAGGSDKYNQALSKYNQALSKYNQALSGGGGSNKKFLLYLAGFVDSDGSIIAQIKPNQHHKFKHALSLTFSVGQKTQRRWFLDKLVDRIGVGHVRDSGSMSEYYLSEIKPLHNFLTQLQPFLKLKQKQANLALKIIEQLPSAKESPDKFLEVCTWVDQIAALNDSKTRKTTSETVRAVLDSLSEKKKSSP

SEQ ID NO: 68 - protein, 354 aa, artificial sequence
  SCOH-ro34-b56-B
MANTKYNEEFLLYLAGFVDGDGSIIAQIKPNQTCKFKHHLSSTFVVTQKTQRRWFLDKLVDEIGVGYVYDSGSVSRYVLSEIKPLHNFLTQLQPFLELKQKQANLVLKIIEQLPSAKESPDKFLEVCTWVDQVAALNDSKTRKTTSETVRAVLDSLSEKKKSSPAAGGSDKYNQALSKYNQALSKYNQALSGGGGSNKKFLLYLAGFVDSDGSIIAQIKPNQHHKFKHALSLTFSVGQKTQRRWFLDKLVDRIGVGHVRDSGSMSEYYLSEIKPLHNFLTQLQPFLKLKQKQANLALKIIEQLPSAKESPDKFLEVCTWVDQIAALNDSKTRKTTSETVRAVLDSLSEKKKSSP

SEQ ID NO: 69 - protein, 354 aa, artificial sequence
  SCOH-ro34-b56-C
MANTKYNEEFLLYLAGFVDGDGSIIAQIKPNQTCKFKHHLSSTFVVTQKTQRRWFLDKLVDEIGVGYVYDSGSVSRYVLSEIKPLHNFLTQLQPFLELKQKQANLVLKIIEQLPSAKESPDKFLEVCTWVDQVAALNDSKTRKTTSETVRAVLDSLSEKKKSSPAAGGSDKYNQALSKYNQALSKYNQALSGGGGSNKKFLLYLAGFVDSDGSIIAQIKPNQHHKFKHALSLTFSVGQKTQRRWFLDKLVDRIGVGHVRDSGSMSEYYLSEIKPLHNFLTQLQPFLKLKQKQANLALKIIEQLPSAKESPDKFLEVCTWVDQVAALNDSKTRKTTSETVRAVLDSLSEKKKSSP

SEQ ID NO: 70 - protein, 354 aa, artificial sequence
  SCOH-ro34-b11-A
MANTKYNEEFLLYLAGFVDGDGSIIAQIKPNQTCKFKHHLSLTFVVTQKTQRRWSLDKLVDEIGVGYVYDSGSVSRYVLSEIKPLHNFLTQLQPFLELKQKQANLVLKIIEQLPSAKESPDKFLEVCTWVDQIAALNDSKTRKTTSETVRAVLDSLSEKKKSSPAAGGSDKYNQALSKYNQALSKYNQALSGGGGSNKKFLLYLAGFVDSDGSIIAQIKPNQHHKFKHALSLTFSVGQKTQRRWFLDKLADRIGVGYVRDSGSMSEYCLSGIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 71 - protein, 354 aa, artificial sequence
  SCOH-ro34-b11-C
MANTKYNEEFLLYLAGFVDGDGSIIAQIKPNQTCKFKHHLSLTFVVTQKTQRRWSLDKLVDEIGVGYVYDSGSVSRYVLSEIKPLHNFLTQLQPFLELKQKQANLVLKIIEQLPSAKESPDKFLEVCTWVDQVAALNDSKTRKTTSETVRAVLDSLSEKKKSSPAAGGSDKYNQALSKYNQALSKYNQALSGGGGSNKKFLLYLAGFVDSDGSIIAQIKPNQHHKFKHALSLTFSVGQKTQRRWFLDKLADRIGVGYVRDSGSMSEYCLSGIKPLHNFLTQLQPFLKLKQKQANLVLKIIEQLPSAKESPDKFLEVCTWVDQVAALNDSKTRKTTSETVRAVLDSLSEKKKSSP

SEQ ID NO: 72 - protein, 354 aa, artificial sequence
  SCOH-ro34-b11-E
MANTKYNEEFLLYLAGFVDGDGSIIAQIKPNQTCKFKHHLSLTFVVTQKTQRRWSLDKLVDEIGVGYVYDSGSVSRYVLSKIKPLHNFLTQLQPFLELKQKQANLVLKIIEQLPSAKESPDKFLEVCTWVDQVAALNDSKTRKTTSETVRAVLDSLSEKKKSSPAAGGSDKYNQALSKYNQALSKYNQALSGGGGSNKKFLLYLAGFVDSDGSIIAQIKPNQHHKFKHALSLTFSVGQKTQRRWFLDKLADRIGVGYVRDSGSMSEYCLSGIKPLHNFLTQLQPFLKLKQKQANLALKIIEQLPSAKESPDKFLEVCTWVDQVAALNDSKTRKTTSETVRAVLDSLSEKKKSSP

SEQ ID NO: 73 - protein, 354 aa, artificial sequence
  SCOH-ro34-b12-A
MANTKYNEEFLLYLAGFVDGDGSIIAQIKPNQTCKFKHHLSLTFVVTQKTQRRWSLDKLVDEIGVGYVYDSGSVSRYVLSEIKPLHNFLTQLQPFLELKQKQANLVLKIIEQLPSAKESPDKFLEVCTWVDQIAALNDSKTRKTTSETVRAVLDSLSEKKKSSPAAGGSDKYNQALSKYNQALSKYNQALSGGGGSNKKFLLYLAGFVDSDGSIIAQIKPNQHHKFKHALSLTFSVGQKTQRRWFLDKLVDRIGVGHVRDSGSMSEYYLSEIKPLHNFLTQLQPFLKLKQKQANLALKIIEQLPSAKESPDKFLEVCTWVDQIAALNDSKTRKTTSETVRAVLDSLSEKKKSSP

SEQ ID NO: 74 - protein, 354 aa, artificial sequence
  SCOH-ro34-b56-C_V2
MANTKYNEEFLLYLAGFVDGDGSIIAQIKPNQTCKFKHHLSSTFVVTQKTQRRWFLDKLVDEIGVGYVYDRGSVSDYVLSEIKPLHNFLTQLQPFLELKQKQANLVLKIIEQLPSAKESPDKFLEVCTWVDQVAALNDSKTRKTTSETVRAVLDSLSEKKKSSPAAGGSDKYNQALSKYNQALSKYNQALSGGGGSNKKFLLYLAGFVDSDGSIIAQIKPNQHHKFKHALSLTFSVGQKTQRRWFLDKLVDRIGVGHVRDSGSMSEYYLSEIKPLHNFLTQLQPFLKLKQKQANLALKIIEQLPSAKESPDKFLEVCTWVDQVAALNDSKTRKTTSETVRAVLDSLSEKKKSSP

SEQ ID NO: 75 - protein, 354 aa, artificial sequence
  SCOH-ro7-b56-C
MANTKYNEEFLLYLAGFVDGDGSIIAQISPNQSSKFKHRLRLTFQVTQKTQRRWLLDKLLDEIGVGYVSDSGSVSNYRLSEIEPLHNFLTQLQPFLELKQKQANLVLKIIEQLPSAKESPDKFLEVCTWVDRVAALNDSKTRKTTSETVRAVLDSLSEKKKSSPAAGGSDKYNQALSKYNQALSKYNQALSGGGGSNKKLLLYLAGFVDSDGSIVAQIKPNQSNKFKHYLRLTLKVTQKTQRRWLLDELVDRIGVGYVYDSGSVSYYQLSEIKPLRSFLAQLQPFLKLKQKQANLVLKIIEQLPSAKESPDKFLEVCTWVDQVAALNDSKTRKTTSETVRAVLDSLGEKKKSSP

SEQ ID NO: 76 - protein, 354 aa, artificial sequence
  SCOH-ro7-b1-C
MANTKYNEEFLLYLAGFADGDGSIIAQISPNQSSKFKHRLRLTFQVTQKTQRRWLLDKLVDEIGVGYVSDSGSVSNYRLSEIEPLHNFLTQLQPFLELKQKQANLVLKIIEQLPSAKESPDKFLEVCTWVDQVAALNDSKTRKTTSETVRAALDSLSEKKKSSPAAGGSDKYNQALSKYNQALSKYNQALSGGGGSNKKLLLYLAGFVDSDGSIIAQIKPNQSSKFKHYLRLTLKVTQKTQRRWLLDELVDRIGVGYVYDSGSVSYYQLSEIKPLHSFLAQLQPFLKLKQKQANLVLKIIEQLPSAKESPDKFLEVCTWVDQVAALNDSKTRKTTSETVHAVLDSLSEKKKSSP

SEQ ID NO: 77 - DNA, 51 bp, Artificial
  34.1 target Cloning oligonucleotide
tggcatacaagtttacttcctcacgctctacgtcaccgcaatcgtctgtca

SEQ ID NO: 78 - protein, 32 aa, Artificial
  RM2 peptidic linker
AAGGSDKYNQALSKYNQALSKYNQALSGGGGS

SEQ ID NO: 79 - DNA, 51 bp, Artificial
  7.1 cloning oligonucleotide
tggcatacaagtttgtcagccaccacacagaaggcagacaatcgtctgtca

SEQ ID NO: 80 - DNA, 22 bp, Artificial
  10AGG_P target
caggacgtcgtacgacgtcctg

SEQ ID NO: 81 - DNA, 22 bp, Artificial
  10CCT_P target
ccctacgtcgtacgacgtaggg

SEQ ID NO: 82 - DNA, 22 bp, Artificial
  5CTT_P target
caaaaccttgtacaaggttttg

SEQ ID NO: 83 - DNA, 22 bp, Artificial
  5CCA_P target
caaaacccagtactgggttttg

SEQ ID NO: 84 - DNA, 22 bp, Artificial
  10CCT-5CTT_P target
ccctaccttgtacaaggtaggg

SEQ ID NO: 85 - DNA, 22 bp, Artificial
  10AGG-5CCA_P target
caggacccagtactgggtcctg

SEQ ID NO: 86 - DNA, 22 bp, Artificial
  Rho31.1 target
tcctcccttttcctggatcctg

SEQ ID NO: 87 - DNA, 22 bp, Artificial
  Rho31.2 target
tcctcccttgtactggatcctg

SEQ ID NO: 88 - DNA, 22 bp, Artificial
  Rho31.3 target
tcctcccttgtacaagggagga

SEQ ID NO: 89 - DNA, 22 bp, Artificial
  Rho31.4 target
caggatccagtactggatcctg

SEQ ID NO: 90 - DNA, 22 bp, Artificial
  Rho31.5 target
tcctcccttttccaagggagga

SEQ ID NO: 91 - DNA, 22 bp, Artificial
  Rho31.6 target
caggatccattcctggatcctg

SEQ ID NO: 92 - protein, 167 aa, artificial sequence
  Rho36.5 I-CreI variant
MANTKYNKEFLLYLAGFVDGDGSIIAQIKPNQGHKFKHQLSLTFRVTQKTQRRWFLDKLVDEIGVGYVYDRGSVSDYWLSEIKPLHNFLTQLQPFLKLKQKQASLVLKIIEQLPSAKESPDKFLEVCTWVDQIAALNDSKTRKTTSETVRAVLDSLSEKKKSSPAAD

SEQ ID NO: 93 - protein, 167 aa, artificial sequence
  Rho36.5 I-CreI variant
MANTKYNKEFLLYLAGFVDGDGSIIAQIKPNQGHKFKHQLSLTFRVTQKTQRRWFLDKLVDEIGVGYVYDRGPVSDYWLSEIKPLHNFLTQLQPFLKLKQKQANLALKIIEQLPSAKESPDKFLEVCTWVDQIAALNDSKTRKTTSETVRAVLDSLSEKKKSSPAAD

SEQ ID NO: 94 - protein, 167 aa, artificial sequence
  Rho36.5 I-CreI variant
MANTKYNKEFLLYLAGFVDGDGSIIAQIKPNQGHKFKHQLSLTFRVTQKTQRRWFLDKLVDEIGVGYVYDRGSVSDYWLSEIKPLHNFLTQLQPFLKLKQKQANLALKIIEQLPSAKESPDKFLEVCTWVDQIAALNDSKTRKTTSETVRAVLDSLSEKKKSSPAAD

SEQ ID NO: 95 - protein, 167 aa, artificial sequence
  Rho36.5 I-CreI variant
MANTKYNKEFLLYLAGFVDGDGSIIAQIKPNQGHKFKHQLSLTFRVTQKTQRRWFLDKLVDEIGVGYVYDRGSVSDYWLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 96 - protein, 167 aa, artificial sequence
  Rho36.5 I-CreI variant
MANTKYNKEFLLYLAGFVDGDGSIIAQIKPNQGHKFKHQLSLTFRVTQKTQRRWFLDKLVDEIGVGYVYDRGSVSDYWLSEIKPLRNFLTQLQPFLKLKQKQANLVLKIIEQLPSAKESPDKFLEVCTWVDQIAALNDSKTRKTTSETVRAVLDSLSEKKKSSPAAD

SEQ ID NO: 97 - protein, 167 aa, artificial sequence
  Rho36.5 I-CreI variant
MANTKYNKEFLLYLAGFVDGDGSIIAQIKPNQGHKFKHQLSLTFRVTQKTQRRWFLDKLVDEIGVGHVYDRGSVSDYWLSEIKPLHNFLTQLQPFLKLKQKQANLVLKIVEQLPSAKESPDKFLEVCTWVDQIAALNDSKTRKTTSETVRAVLDSLSEKKKSSPAAD

SEQ ID NO: 98 - protein, 167 aa, artificial sequence
  Rho36.5 I-CreI variant
MANTKYNKEFLLYLAGFVDGDGSIIAQIKPNQGHKFKHQLSLTFRVTQKTQRRWFLDKLVDEIGVGYVYDRGSVSDYWLSEIKPLHNFLTQLQPFLKLKQKQANLVLKIIEQLPSARESPDKFLEVCTWVDQIAALNDSKTRKTTSETVRAVLDSLSEKKKSSPAAD

SEQ ID NO: 99 - protein, 167 aa, artificial sequence
  Rho36.5 I-CreI variant
MANTKYNKEFLLYLAGFVDGDGSIIAQIKPNQGHKFKHQLSLTFRVTQKTQRRWFLDKLVDEIGVGYVYDRGSVSDYWLSEIKPLHNFLTQLQPFLKLKQKQANLVLKIIEQLPSAKESPDRFLEVCTWVDQIAALNDSKTRKTTSETVRAVLDSLSEKKKSSPAAD

SEQ ID NO: 100 - protein, 167 aa, artificial sequence
  Rho36.5 I-CreI variant
MANTKYNKEFLLYLAGFVDGDGSIIAQIKPNRGHKFKHQLSLTFRVTQKTQRRWFLDKLVDEIGVGYVYDRGSVSDYWLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 101 - protein, 167 aa, artificial sequence
  Rho36.6 I-CreI variant
MANTKYNKEFLLYLAGFVDGDGSIIAQIKPNQSSKFKHYLSLTFRVTQKTQRRWFLDRLVDEIGVGHVYDSGSVSNYTLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 102 - protein, 167 aa, artificial sequence; identical to SEQ ID NO: 101
  Rho36.6 I-CreI variant

SEQ ID NO: 103 - protein, 167 aa, artificial sequence
  Rho36.6 I-CreI variant
MANTKYNKEFLLYLAGFVDGDGSIIAQIKPNQSSKFKHYLSLTFRVTQKTQRRWFLDRLVDEIGVGHVYDSRSVSNYTLSEIKPLHNLLTQLQPFLKLKQKQANLALKIIEQLPSAKESPDKFLEVCTWVDQIAALNDSKTRKTTSETVRAVLDSLSEKKKSSPAAD

SEQ ID NO: 104 - protein, 354 aa, artificial sequence
  SCOH-Ro36-b1-C-pCLS5645
MANTKYNEEFLLYLAGFVDGDGSIIAQIKPNQGHKFKHQLSLTFRVTQKTQRRWFLDKLVDEIGVGYVYDRGPVSDYWLSEIKPLHNFLTQLQPFLELKQKQANLALKIIEQLPSAKESPDKFLEVCTWVDQVAALNDSKTRKTTSETVRAVLDSLSEKKKSSPAAGGSDKYNQALSKYNQALSKYNQALSGGGGSNKKFLLYLAGFVDSDGSIIAQIKPNQSSKFKHYLSLTFRVTQKTQRRWFLDRLVDRIGVGHVYDSRSVSNYTLSEIKPLHNLLTQLQPFLKLKQKQANLALKIIEQLPSAKESPDKFLEVCTWVDQVAALNDSKTRKTTSETVRAVLDSLSEKKKSSP

SEQ ID NO: 105 - protein, 167 aa, artificial sequence
  Rho31.5 I-CreI variant
MANTRYNKGFLLYLAGFVDGDGSIIAQIKPNQSSKFKHYLSLTFRVTQKTQRRWFLDKLVDEIGVGYVYDSGSVSDYNLSEIKPLHNLLTQLQPFLKLKQKQANLALKIIEQLPSAKESPDKFLEVCTWVDQIAALNDSKTRKTTSETVRAVLDSLSEKKRPSPAAD

SEQ ID NO: 106 - protein, 167 aa, artificial sequence
  Rho31.5 I-CreI variant
MANTKYNKEFLLYLAGFVDGDGSIIAQIKPNQSSKFKHYLSLTFRVTQKTQRRWFLDKLVDEIGVGYVYDSGSVSDYNLSEIKPLRNLLTQLQPFLKLKQKQANLVLKIIEQLPSAKESPDKFLEVCTWVDQIAALNDSKTRKTTSETVRAVLDSLSEKKKPSPAAD

SEQ ID NO: 107 - protein, 167 aa, artificial sequence
  Rho31.5 I-CreI variant
MANTKYNKEFLLYLAGFVDGDGSIIAQIKPNQSSKFKHYLSLTFRVTQKTQRRWFLDKLVDEIGVGHVYDSGSVSDYNLSEIKPLHNFLAQLQPFLKLKQKQANLVLKIIEQLPSAKESPDKFLEVCTWVDQIAALNDSKTRKTTSETVRAVLDSLSGEKKSSPAAD

SEQ ID NO: 108 - protein, 167 aa, artificial sequence
  Rho31.5 I-CreI variant
MANTKYNKEFLLYLAGFVDGDGSIIAQIKPNQSSKFKHYLSLTFRVTQKTQRRWFLDKLVDEIGVGYVYDSGSVSDYNLSEIKPLHNLLTQLQPFLKLKQKQANLVLKIIEQLPSAKESPGKFLEVCTWVDQIAALNDSKTRKTTSETVRAVLDSLSEKKKPSPAAD

SEQ ID NO: 109 - protein, 167 aa, artificial sequence
  Rho31.5 I-CreI variant
MANTKYNKEFLLYLAGFVDGDGSIIAQIKPNQSSKFKHYLSLTFRVTQKTQRRWFLDKLVDEIGVGHVYDSGSVSDYNLSEIKPLHNLLTQLQPLLKLKQKQANLVLKIIEQLPSAKESPDKFLEVCTWVDQIAALNDSKTRKTTSETVRAVLDSLSGKKKSSPAAD

SEQ ID NO: 110 - protein, 167 aa, artificial sequence
  Rho31.5 I-CreI variant
MANTKYSKEFLLYLAGFVDGDGSIIAQIKPNQSSKFKHYLSLTFRVTQKTQRRWFLDKLVDEIGVGHVYDSGSVSDYNLSEIKPLHNFLAQLQPFLKLKQKQANLVLKIIEQLPSAKESPDKFLEVCTWVDQIAALNDSKTRKTTSETVRAVLDSLSGKKKPSPAAD

SEQ ID NO: 111 - protein, 167 aa, artificial sequence
  Rho31.5 I-CreI variant
MANTKYNKEFLLYLAGFVDGDGSIIAQIKPNQSSKFKHYLSLTFRVTQKTQRRWFLDKLVDEIGVGYVYDTGSVSDYNLSEIKPLHNLLTQLQPFLKLKQKQANLVLKIIEQLPSAKESPDKFLEVCTWVDQIAALNDSKTRKTTSETVRAVLVSLSEKKKPSPAAD

SEQ ID NO: 112 - protein, 167 aa, artificial sequence
  Rho31.5 I-CreI variant
MANTKYNKEFLLYLAGFVDGDGSIIAQIKPNQSSKFKHYLSLTFRVTQKTQRRWFLDKLVDEIGVGYVYDSGSVSDYNLSEIKPLHNLLTQLQPFLKLKQKQANLVLKIIEQLPSAKESPDKFLEVCTWMDQIAALNDSKTRKTTSETVRAVLDSLSEKKKPSPAAD

SEQ ID NO: 113 - protein, 167 aa, artificial sequence
  Rho31.5 I-CreI variant
MANTKYSKEFLLYLAGFVDGDGSIIAQIKPNQSSKFKHYLSLTFRVTQKTQRRWFLDKLVDEIGVGHVYDSGSVSDYNLSEIKPLHNFLAQLQPFLKLKQKQANLVLKIIEQLPSAKESPDKFLEVCTWVDQIAALNDSKTRKTTSETVRAVLDSLSGKKKSSPAAD

SEQ ID NO: 114 - protein, 167 aa, artificial sequence
  Rho31.6 I-CreI variant
MANTKYNKEFLLYLAGFVDGDGSIIAQIEPNQSYKFKHRLKLTLKVTQKTQRRWLLDKLVDEIGVGYVRDEGSVSNYILSEVKPLHNFLTQLQPFLRLKQKQANLVLKIIEQLPSAKESPDKFLEVCTWVDQIAALNDSKTRKTTSETVRAVLVSLSEKKGSSPAAD

SEQ ID NO: 115 - protein, 167 aa, artificial sequence
  Rho31.6 I-CreI variant
MAITKYNKEFLLYLAGFVDGDGSIIAQIEPNQSYKFKHRLKLTLKVTQKTQRRWLLDKLVDEIGVGYVRDEGSVSNYILSEVKPLHNFLTQLQPFLRLKQKQANLVLKIIEQLPSAKESPDKFLEVCTWVDQIAALNDSKTRKTTSETVRAVLVSLSEKKRSSPAAD

SEQ ID NO: 116 - protein, 167 aa, artificial sequence
  Rho31.6 I-CreI variant
MANTKYNKEFLLYLAGFVDGDGSIIAQIKPNQSSKFKHYLSLTLRVTQKTQRRWFLDKLVDEIGVGYVYDSGSVSDYNLSEIKPLHNLLTQLQPFLKLKQKQANLVLKIIEQLPSAKESPDKFLEVCTWVDQIAALNDSKTRKTTSETVRAVLDSLSEKKKPSPAAD

SEQ ID NO: 117 - protein, 354 aa, artificial sequence
  SCOH-ro31-b56-A-pCLS6298
MANTKYNEEFLLYLAGFVDGDGSIIAQIKPNQSSKFKHYLSLTFRVTQKTQRRWFLDKLVDEIGVGYVYDSGSVSDYNLSEIKPLRNLLTQLQPFLELKQKQANLVLKIIEQLPSAKESPDKFLEVCTWVDQIAALNDSKTRKTTSETVRAVLDSLSEKKKPSPAAGGSDKYNQALSKYNQALSKYNQALSGGGGSNKKFLLYLAGFVDSDGSIIAQIEPNQSYKFKHRLKLTLKVTQKTQRRWLLDKLVDRIGVGYVRDEGSVSNYILSEVKPLHNFLTQLQPFLRLKQKQANLVLKIIEQLPSAKESPDKFLEVCTWVDQIAALNDSKTRKTTSETVRAVLVSLSEKKRSSP

SEQ ID NO: 118 - protein, 354 aa, artificial sequence
  SCOH-ro31-b56-D-pCLS6299
MANTKYNEEFLLYLAGFVDGDGSIIAQIKPNQSSKFKHYLSLTFRVTQKTQRRWFLDKLVDEIGVGYVYDSGSVSDYNLSEIKPLRNLLTQLQPFLELKQKQANLVLKIIEQLPSAKESPDKFLEVCTWVDQIAALNDSKTRKTTSETVRAVLDSLSEKKKPSPAAGGSDKYNQALSKYNQALSKYNQALSGGGGSNKKFLLYLAGFVDSDGSIIAQIEPNQSYKFKHRLKLTLKVTQKTQRRWLLDKLVDRIGVGYVRDEGSVSNYILSEVKPLHNFLTQLQPFLRLKQKQANLVLKIIEQLPSAKESPDKFLEVCTWVDQVAALNDSKTRKTTSETVRAVLVSLSEKKRSSP

SEQ ID NO: 119 - protein, 354 aa, artificial sequence
  SCOH-ro31-b1-A-pCLS6300
MANTKYNEEFLLYLAGFVDGDGSIIAQIKPNQSSKFKHYLSLTFRVTQKTQRRWFLDKLVDEIGVGHVYDSGSVSDYNLSEIKPLHNLLTQLQPLLELKQKQANLVLKIIEQLPSAKESPDKFLEVCTWVDQIAALNDSKTRKTTSETVRAVLDSLSGKKKSSPAAGGSDKYNQALSKYNQALSKYNQALSGGGGSNKKFLLYLAGFVDSDGSIIAQIEPNQSYKFKHRLKLTLKVTQKTQRRWLLDKLVDRIGVGYVRDEGSVSNYILSEVKPLHNFLTQLQPFLRLKQKQANLVLKIIEQLPSAKESPDKFLEVCTWVDQIAALNDSKTRKTTSETVRAVLVSLSEKKGSSP

SEQ ID NO: 120 - protein, 354 aa, artificial sequence
  SCOH-ro31-b1-B-pCLS6301
MANTKYNEEFLLYLAGFVDGDGSIIAQIKPNQSSKFKHYLSLTFRVTQKTQRRWFLDKLVDEIGVGHVYDSGSVSDYNLSEIKPLHNLLTQLQPLLELKQKQANLVLKIIEQLPSAKESPDKFLEVCTWVDQVAALNDSKTRKTTSETVRAVLDSLSGKKKSSPAAGGSDKYNQALSKYNQALSKYNQALSGGGGSNKKFLLYLAGFVDSDGSIIAQIEPNQSYKFKHRLKLTLKVTQKTQRRWLLDKLVDRIGVGYVRDEGSVSNYILSEVKPLHNFLTQLQPFLRLKQKQANLVLKIIEQLPSAKESPDKFLEVCTWVDQIAALNDSKTRKTTSETVRAVLVSLSEKKGSSP

SEQ ID NO: 121 - protein, 354 aa, artificial sequence
  pCLS6302-SCOH-ro31-b1-D-pCLS6302
MANTKYNEEFLLYLAGFVDGDGSIIAQIKPNQSSKFKHYLSLTFRVTQKTQRRWFLDKLVDEIGVGHVYDSGSVSDYNLSEIKPLHNLLTQLQPLLELKQKQANLVLKIIEQLPSAKESPDKFLEVCTWVDQIAALNDSKTRKTTSETVRAVLDSLSGKKKSSPAAGGSDKYNQALSKYNQALSKYNQALSGGGGSNKKFLLYLAGFVDSDGSIIAQIEPNQSYKFKHRLKLTLKVTQKTQRRWLLDKLVDRIGVGYVRDEGSVSNYILSEVKPLHNFLTQLQPFLRLKQKQANLVLKIIEQLPSAKESPDKFLEVCTWVDQVAALNDSKTRKTTSETVRAVLVSLSEKKGSSP

SEQ ID NO: 122 - protein, 354 aa, artificial sequence
  SCOH-ro31-b56-B-pCLS6304
MANTKYNEEFLLYLAGFVDGDGSIIAQIKPNQSSKFKHYLSLTFRVTQKTQRRWFLDKLVDEIGVGYVYDSGSVSDYNLSEIKPLRNLLTQLQPFLELKQKQANLVLKIIEQLPSAKESPDKFLEVCTWVDQVAALNDSKTRKTTSETVRAVLDSLSEKKKPSPAAGGSDKYNQALSKYNQALSKYNQALSGGGGSNKKFLLYLAGFVDSDGSIIAQIEPNQSYKFKHRLKLTLKVTQKTQRRWLLDKLVDRIGVGYVRDEGSVSNYILSEVKPLHNFLTQLQPFLRLKQKQANLVLKIIEQLPSAKESPDKFLEVCTWVDQIAALNDSKTRKTTSETVRAVLVSLSEKKRSSP

SEQ ID NO: 123 - protein, 354 aa, artificial sequence
  SCOH-ro31-b1-E-pCLS6316
MANTKYNEEFLLYLAGFVDGDGSIIAQIKPNQSSKFKHYLSLTFRVTQKTQRRWFLDKLVDEIGVGHVYDSGSVSDYNLSKIKPLHNLLTQLQPLLELKQKQANLVLKIIEQLPSAKESPDKFLEVCTWVDQVAALNDSKTRKTTSETVRAVLDSLSGKKKSSPAAGGSDKYNQALSKYNQALSKYNQALSGGGGSNKKFLLYLAGFVDSDGSIIAQIEPNQSYKFKHRLKLTLKVTQKTQRRWLLDKLVDRIGVGYVRDEGSVSNYILSEVKPLHNFLTQLQPFLRLKQKQANLALKIIEQLPSAKESPDKFLEVCTWVDQVAALNDSKTRKTTSETVRAVLVSLSEKKGSSP